(19) United States
(12) Patent Application Publication
(10) Pub. No.: US 2025/0235265 A1
(43) Pub. Date: Jul. 24, 2025

(54) SYSTEMS, DEVICES, AND METHODS FOR PREDICTING TOTAL KNEE ARTHROPLASTY AND PARTIAL KNEE ARTHROPLASTY PROCEDURES

(71) Applicant: Mako Surgical Corporation, Weston, FL (US)

(72) Inventors: Sietske WITVOET, Delfgauw (NL); Alison LONG, Weston, FL (US); Jared Richard WEIR, Saginaw, MI (US); Alan BRETT, Weston, FL (US); Niall MAGUIRE, Weston, FL (US)

(73) Assignee: Mako Surgical Corporation, Weston, FL (US)

(21) Appl. No.: 19/032,768

(22) Filed: Jan. 21, 2025

Related U.S. Application Data
(60) Provisional application No. 63/725,156, filed on Nov. 26, 2024.
(60) Provisional application No. 63/709,057, filed on Oct. 18, 2024.
(60) Provisional application No. 63/624,932, filed on Jan. 25, 2024.
(60) Provisional application No. 63/623,553, filed on Jan. 22, 2024.

(51) Int. Cl. A61B 34/10; A61B 34/00; A61B 90/00; G06T 7/00; G16H 10/60; G16H 50/30; G16H 50/70

(52) U.S. Cl. CPC A61B 34/10; A61B 34/25; A61B 90/37; G06T 7/0012; G16H 10/60; G16H 50/30; G16H 50/70; A61B 2034/105; A61B 2090/374; A61B 2090/3762; G06T 2207/10072; G06T 2207/10088; G06T 2207/20081; G06T 2207/30008

(57) ABSTRACT

A method for predicting a type of surgery required for a patient comprises: receiving imaging data including at least one image acquired of a patient's anatomy; determining at least one parameter of the patient's anatomy based on the image data, the at least one parameter including at least one of a B-score and a C-score; receiving patient data regarding the patient; generating, based at least in part on the at least one parameter and the patient data, a predicted procedure for the patient; and outputting the predicted procedure for display within a graphical user interface (GUI).

### CROSS-REFERENCE TO RELATED APPLICATION(S)

This patent application is a continuation of and claims the benefit of priority to U.S. Provisional Application No. 63,725,156 filed on Nov. 26, 2024, 63/709,057, filed on Oct. 18, 2024, 63,624,932, filed Jan. 25, 2024, and 63/623,553, filed Jan. 22, 2024, all of which are incorporated herein by reference in their entireties.

### FIELD OF THE DISCLOSURE

The present disclosure relates to systems and methods for optimizing medical procedures, and in particular to a system and a method for processing and displaying images to provide clinical decision intelligence and to optimize outcomes after joint replacement procedures.

### BACKGROUND OF THE DISCLOSURE

Musculoskeletal disease presents unique problems for medical practitioners. Surgeries incorporating prosthetics and/or implants such as joint replacement procedures often require careful consideration of various factors. Improved systems and methods for performing, collecting, and analyzing or processing image acquisition data are desired.

### BRIEF SUMMARY OF THE DISCLOSURE

In an aspect of the present disclosure, a method for predicting a type of surgery required for a patient may include: receiving imaging data including at least one image acquired of a patient's anatomy; determining at least one parameter of the patient's anatomy based on the image data, the at least one parameter including at least one of a B-score and a C-score; receiving patient data regarding the patient; generating, based at least in part on the at least one parameter and the patient data, a predicted procedure for the patient; and outputting the predicted procedure for display within a graphical user interface (GUI). In some instances, the predicted procedure is either a partial knee arthroplasty or a total knee arthroplasty. In some instances, generating the predicted procedure includes determining a type of implant for the patient. In some instances, predicted procedure is generated at least in part using a machine learning model trained with a plurality of predicted procedures generated for prior patients and a respective plurality of confirmed procedures received for the prior patients. In some instances, the method further includes receiving a confirmed procedure for the patient and using the predicted procedure and the confirmed procedure to further train the machine learning model. In some instances, the confirmed procedure is received through the GUI. In some instances, the at least one parameter includes a B-score and a C-score. In some instances, the at least one parameter includes a plurality of C-scores. In some instances, the plurality of C-scores includes two or more of a medial tibiofemoral C-score, a lateral tibiofemoral C-score, a medial patellofemoral C-score, and a lateral patellofemoral C-score. In some instances, the method further includes normalizing the patient data.

In another aspect of the present disclosure, a system for predicting a type of surgery required for a patient includes: an imaging device operative to generate imaging data including at least one image acquired of a patient's anatomy; and at least one processor executed on at least one computing device and operative to: receive the imaging data including the at least one image acquired of the patient's anatomy generated by the imaging device; determine at least one parameter of the patient's anatomy based on the imaging data, the at least one parameter including at least one of a B-score and a C-score; receive patient data regarding the patient; generate, based at least in part on the at least one parameter and the patient data, a predicted procedure for the patient; and output the predicted procedure for display within a graphical user interface (GUI). In some instances, the predicted procedure is either a partial knee arthroplasty or a total knee arthroplasty. In some instances, the at least one processor is further operative to generate the predicted procedure by determining a type of implant for the patient. In some instances, the at least one processor is further operative to generate the predicted procedure at least in part using a machine learning model trained with a plurality of predicted procedures generated for prior patients and a respective plurality of confirmed procedures received for the prior patients. In some instances, the at least one processor is further operative to receive a confirmed procedure for the patient and use the confirmed procedure further train the machine learning model. In some instances, the at least one processor is further operative to receive the confirmed procedure through the GUI. In some instances, the at least one parameter includes a B-score and a C-score. In some instances, the at least one parameter includes a plurality of C-scores. In some instances, the plurality of C-scores includes two or more of a medial tibiofemoral C-score, a lateral tibiofemoral C-score, a medial patellofemoral C-score, and a lateral patellofemoral C-score. In some instances, the at least one processor is further operative to normalize the patient data.

### BRIEF DESCRIPTION OF THE DRAWINGS

A more complete appreciation of the subject matter of this disclosure and the various advantages thereof may be understood by reference to the following detailed description, in which reference is made to the following accompanying drawings:

FIG. 1 is a schematic diagram depicting an electronic data processing system having an image analysis system, according to aspects of this disclosure.

FIG. 2 is a schematic diagram of the electronic data processing system of FIG. 1 depicting interactions among preoperative measurement systems, preoperative data, the image analysis system, outputs, and output systems, according to aspects of this disclosure.

FIGS. 3A and 3B illustrate exemplary imaging data by the image analysis system and exemplary graphical user interfaces (GUIs) to display the imaging data, according to aspects of this disclosure.

FIGS. 4A through 4J illustrate exemplary GUIs or user interfaces showing models of a patient anatomy, an implant, and related determined parameters or analysis, according to aspects of this disclosure.

FIG. 5 illustrates exemplary GUIs depicting joint-space width between two bones of a patient's anatomy, according to aspects of this disclosure.

FIGS. 6A through 6C illustrate exemplary GUIs depicting predicted cartilage loss based on joint-space width, according to aspects of this disclosure.

FIG. 7 illustrates exemplary GUIs depicting osteophytes on an acquired image and/or a representative model of a patient's anatomy, according to aspects of this disclosure.

FIG. 8 illustrates exemplary GUIs depicting osteophytes in connection with a segmentation process, according to aspects of this disclosure.

FIG. 9 illustrates exemplary GUIs depicting osteophytes in different views, according to aspects of this disclosure.

FIG. 10 is a flow chart depicting an exemplary method to determine osteophyte volume, according to aspects of this disclosure.

FIGS. 11A-11E illustrate an exemplary GUI depicting anatomical compartments of one or more bones in a patient's anatomy, according to aspects of this disclosure.

FIG. 12 is a flow chart depicting an exemplary method to determine compartmental osteophyte volumes, according to aspects of this disclosure.

FIG. 13 illustrates exemplary GUIs depicting osteophytes according to compartments, according to aspects of this disclosure.

FIG. 14 illustrates an exemplary GUI depicting a bone shape or B-score progression, according to aspects of this disclosure.

FIG. 15 illustrates an exemplary GUI depicting a bone shape according to a B-score and related predicted outcomes based on B-score, according to aspects of this disclosure.

FIGS. 16A through 16C illustrate exemplary GUIs depicting a bone shape progression according to a B-score and related predicted outcomes based on B-score, according to aspects of this disclosure.

FIG. 17 is a flow chart depicting an exemplary method to determine tissue-to-bone ratio, according to aspects of this disclosure.

FIG. 18 illustrates exemplary GUIs in connection with a segmentation and/or thresholding process to determine tissue-to-bone ratio, according to aspects of this disclosure.

FIG. 19 illustrates an exemplary GUI visually depicting a tissue-to-bone ratio, according to aspects of this disclosure.

FIG. 20 illustrates an exemplary GUI configured to allow toggling osteophytes on and off, according to aspects of this disclosure.

FIG. 21 illustrates an exemplary GUI configured to allow toggling a portion of a bone on and off.

FIG. 22 illustrates an exemplary GUI configured to allow changing an opacity of osteophytes.

FIG. 23 illustrates an exemplary GUI configured to allow toggling osteophytes and/or various bones on and off.

FIG. 24 illustrates an exemplary GUI configured to show a simulated movement of a patient's anatomy, according to aspects of this disclosure.

FIG. 25 illustrates an exemplary GUI depicting a model of a planned implant overlaid onto an acquired image and a representative model, according to aspects of this disclosure.

FIG. 26 illustrates an exemplary GUI depicting bone resection planes, according to aspects of this disclosure.

FIG. 27 illustrates an exemplary GUI depicting a virtual bone model, according to aspects of this disclosure.

FIGS. 28A and 28B illustrate an exemplary GUI depicting planned bone cuts on a virtual bone model, according to aspects of this disclosure.

FIG. 29A is a schematic diagram of the electronic data processing system of FIG. 1 depicting interactions among intraoperative measurement systems, intraoperative data, the image analysis system, outputs, and output systems, according to aspects of this disclosure.

FIG. 29B is an exemplary method of generating GUIs based on acquired images, according to aspects of this disclosure.

FIG. 30 illustrates an exemplary method for deciding between surgical and non-surgical treatments, according to aspects of this disclosure.

FIG. 31 illustrates an exemplary method for making treatment or surgical decisions, according to aspects of this disclosure.

FIG. 32 illustrates an exemplary method for making treatment or surgical decisions, according to aspects of this disclosure.

FIG. 33 illustrates an exemplary method for making treatment or surgical decisions, according to aspects of this disclosure.

FIG. 34 illustrate an exemplary GUI depicting planned bone cuts on a virtual bone model with an implant, according to aspects of this disclosure.

FIG. 35 illustrate exemplary GUIs depicting predicted cartilage loss based on joint-space width, according to aspects of this disclosure.

FIG. 36 illustrates an exemplary GUI depicting a density of a bone volume, according to aspects of this disclosure.

FIG. 37 depicts a predicted procedure algorithm implemented as a decision tree model, according to aspects of this disclosure.

FIG. 38 illustrates an exemplary GUI depicting a predicted procedure, according to aspects of this disclosure.

FIG. 39 is an exemplary method for generating a predicted procedure for a patient, according to aspects of this disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “implant trial” and “trial” will be used interchangeably and as such, unless otherwise stated, the explicit use of either term is inclusive of the other term. In this disclosure, “user” is synonymous with “practitioner” and may be any person completing the described action (e.g., surgeon, technician, nurse, etc.).

An implant may be a device that is at least partially implanted in a patient and/or provided inside of a patient's body. For example, an implant may be a sensor, artificial bone, or other medical device coupled to, implanted in, or at least partially implanted in a bone, skin, tissue, organs, etc. A prosthesis or prosthetic may be a device configured to assist or replace a limb, bone, skin, tissue, etc., or portion thereof. Many prostheses are implants, such as a tibial prosthetic component. Some prostheses may be exposed to an exterior of the body and/or may be partially implanted, such as an artificial forearm or leg. Some prostheses may not be considered implants and/or otherwise may be fully exterior to the body, such as a knee brace. Systems and methods disclosed herein may be used in connection with implants, prostheses that are implants, and also prostheses that may not be considered to be “implants” in a strict sense. Therefore, the terms “implant” and “prosthesis” will be used interchangeably and as such, unless otherwise stated, the explicit use of either term is inclusive of the other term. Although the term “implant” is used throughout the disclosure, this term should be inclusive of prostheses which may not necessarily be “implants” in a strict sense.

In describing preferred embodiments of the disclosure, reference will be made to directional nomenclature used in describing the human body. It is noted that this nomenclature is used only for convenience and that it is not intended to be limiting with respect to the scope of the invention. For example, as used herein, the term “proximal” means toward the human body and/or away from the operator, and the term “distal” means away from the human body and/or towards the operator. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such system, process, method, article, or apparatus. The term “exemplary” is used in the sense of “example,” rather than “ideal.” Further, relative terms such as, for example, “about,” “substantially,” “approximately,” etc., are used to indicate a possible variation of +10% in a stated numeric value or range.

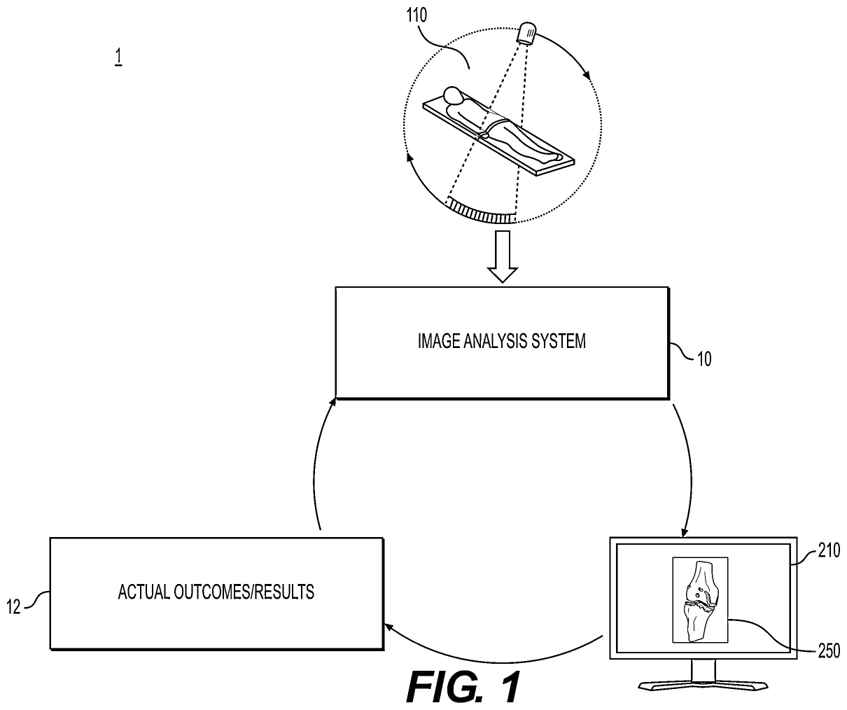

FIG. 1 illustrates an electronic data processing system 1 for collecting, storing, processing, and outputting data during a course of treatment of a patient.

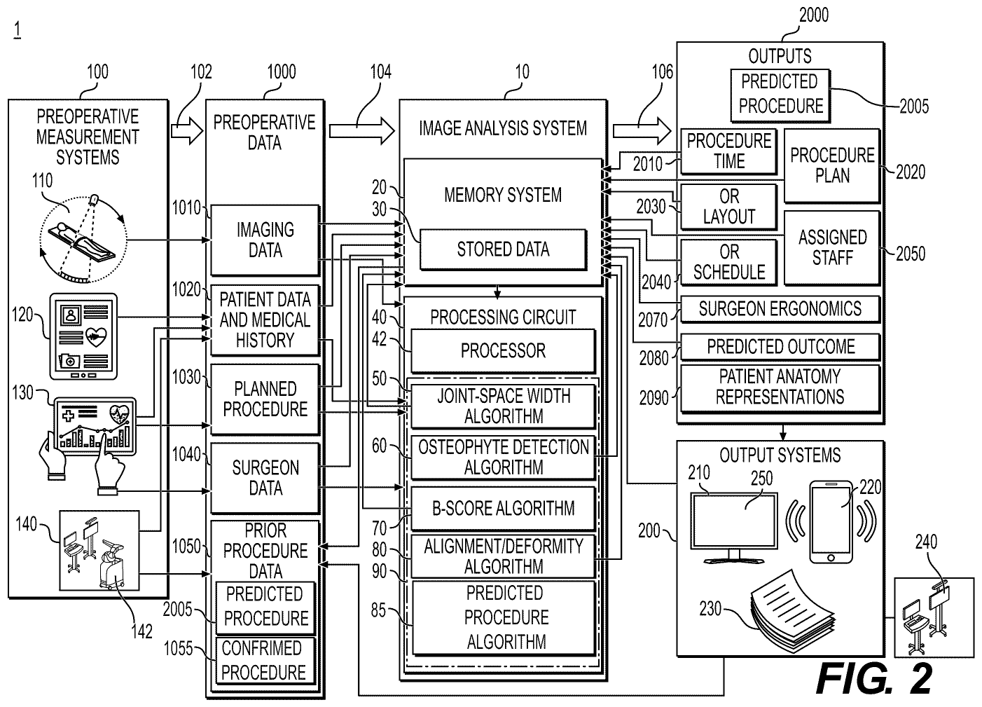

Referring to FIG. 1, the electronic data processing system 1 may include a diagnostic imaging device 110 (e.g., a computed tomography or CT scanner), an image analysis system 10, and an electronic display 210. An instant patient who is planning to undergo a procedure (e.g., surgery) may first undergo imaging using the diagnostic imaging device 110. The image analysis system 10 may analyze images and/or information collected during imaging (which may be transmitted from or stored in the device 110) to determine various outputs 2000 (FIG. 2) and generate graphical user interfaces (GUIs) 250 to display on a display 210. The image analysis system 10 may further determine recommended procedures (e.g., a predicted procedure), procedure logistics (e.g., procedure scheduling), and/or predicted outcomes (e.g., a risk of complication during the procedure or a risk of infection post-procedure) that are based on the determined outputs 2000. As the course of treatment is continued, actual outcomes and/or results 12 may also be used by the image analysis system 10 to either update its predictions and/or to make future predictions for future patients. The image analysis system 10 may be implemented as one or more computer systems or cloud-based electronic processing systems. Details of the image analysis system 10 are discussed with reference to FIG. 2.

Referring to FIG. 2, the electronic data processing system 1 may include one or more preoperative measurement systems 100 which collect and/or output (via arrow 102) preoperative data 1000 about the instant patient and/or prior patients (e.g., similar prior patients). The image analysis system 10 may receive (via arrow 104) and analyze the preoperative data 1000 to generate one or more outputs or determinations 2000, which may be output (via arrow 106) to one or more output systems 200.

### Preoperative Measurement Systems 100

The preoperative measurement systems 100 may include the imaging device 110, electronic devices storing electronic medical records (EMR) 120; patient, practitioner, and/or user interfaces or applications 130 (such as on tablets, computers, or other mobile devices); and a robotic and/or automated data system or platform 140 (e.g., MAKO Robot System or platform, MakoSuite, etc.), which may include a robotic device 142. The electronic data processing system 1 may collect current imaging data 1010 via the imaging device 110 and supplemental or additional information (e.g., patient data and medical history 1020, planned procedure data 1030, surgeon and/or staff data 1040, and/or prior procedure data 1050) via EMR 120, interfaces 130, sensors and/or electronic medical devices, and/or robotic platform 140. Each of the devices in the preoperative measurement systems 100 (e.g., the imaging device 110, EMR 120, user interfaces or applications 130, sensors and/or electronic medical devices, and robotic platform 140) may include one or more communication modules (e.g., WiFi modules, BlueTooth modules, etc.) configured to transmit preoperative data 1000 to each other, to the image analysis system 10, and/or to the one or more output systems 200.

The imaging device 110 may be configured to collect or acquire one or more images, videos, or scans of a patient's internal anatomy, such as bones, ligaments, soft tissues, brain tissue, etc., to generate imaging data 1010, which will be described in more detail later. The imaging device 110 may include a computed tomography (CT) scanner (e.g., a supine CT scanner). The imaging device 110 may include, in addition to a CT scanner, a magnetic resonance imaging (MRI) machine, an x-ray machine, a radiography system, an ultrasound system, a thermography system, a tactile imaging system, an elastography, nuclear medicine functional imaging system, a positron emission tomography (PET) system, a single-photon emission computer tomography (SPECT) system, a camera, etc. The collected images, videos, or scans may be transmitted, automatically or manually, to the image analysis system 10. In some examples, a user may select specific images from a plurality of images taken with an imaging device 110 to be transmitted to the image analysis system 10.

The electronic data processing system 1 may use previously collected data from EMR 120, which may include patient data and medical history 1020 in the form of past practitioner assessments, medical records, past patient reported data, past imaging procedures, treatments, etc. For example, EMR 120 may contain data on demographics, medical history, biometrics, past procedures, general observations about the patient (e.g., mental health), lifestyle information, data from physical therapy, etc. Patient data and medical history 1020 will be described in further detail below.

The electronic data processing system 1 may also collect present or current (e.g., in real time) patient data via patient, practitioner, and/or user interfaces or applications 130. These user interfaces 130 may be implemented on mobile applications and/or patient management websites or interfaces, such as OrthologIQ®. User interfaces 130 may present questionnaires, surveys, or other prompts for practitioners or patients to enter assessments (e.g., throughout a prehabilitation program prior to a procedure), observed psychosocial information and/or readiness for surgery, comments, etc. for additional patient data 1020. Patients may also enter psychosocial information such as perceived or evaluated pain, stress level, anxiety level, feelings, and other patient reported outcome measures (PROMS) into these user interfaces 130. Patients and/or practitioners may report lifestyle information via user interfaces 130. User interfaces 130 may also collect clinical data such as planned procedure 1030 data and planned surgeon and/or staff data 1040 described in more detail later. These user interfaces 130 may be executed on and/or combined with other devices disclosed herein (e.g., with robotic platform 140).

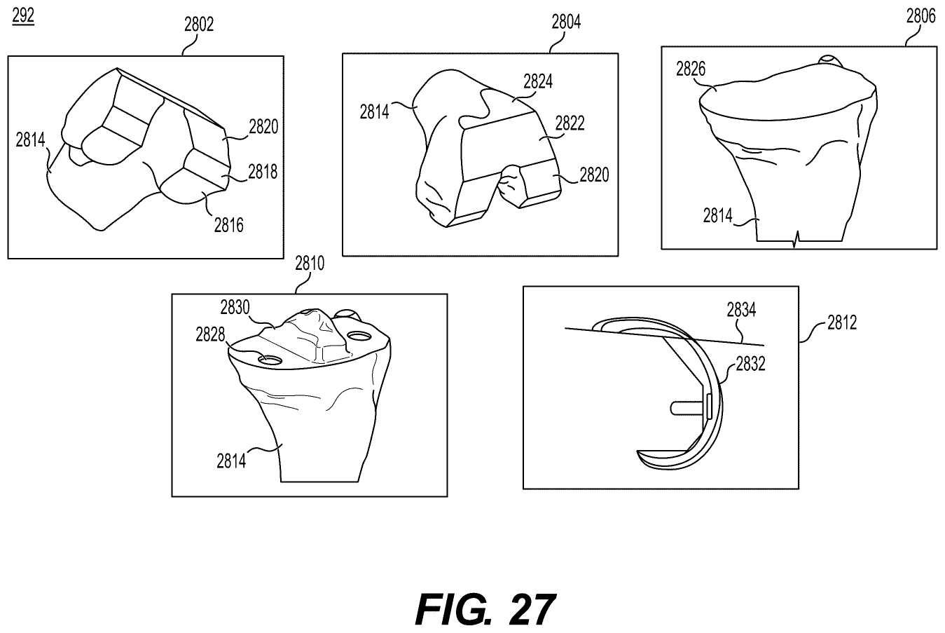

The electronic data processing system 1 may collect prior procedure data 1050 from prior patients and/or other real-time data or observations (e.g., observed patient data 1020) via robotic platform 140. The robotic platform 140 may include one or more robotic devices (e.g., surgical robot 142), computers, databases, etc. used in prior procedures with different patients. The surgical robot 142 may have assisted with, via automated movement, surgeon assisted movement, and/or sensing, a prior procedure and may be implemented as or include one or more automated or robotic surgical tools, robotic surgical or Computerized Numerical Control (CNC) robots, surgical haptic robots, surgical tele-operative robots, surgical hand-held robots, or any other surgical robot. The surgical robot 142 will be described in more detail with reference to FIG. 27.

Although the preoperative measurement system(s) 100 is described in connection with imaging device 110, EMR 120, user interfaces 130, and robotic platform 140, other devices may be used preoperatively to collect preoperative data 1000. For example, mobile devices such as cell phones and/or smart watches may include various sensors (e.g., gyroscopes, accelerometers, temperature sensors, optical or light sensors, magnetometer, compass, global positioning systems (GPS) etc.) to collect patient data 1020 such as location data, sleep patterns, movement data, heart rate data, lifestyle data, activity data, etc. As another example, wearable sensors, heart rate monitors, motion sensors, external cameras, etc. having various sensors (e.g., cameras, optical light sensors, barometers, GPS, accelerometers, temperature sensors, pressure sensors, magnetometer or compass, MEMs devices, inclinometers, acoustical ranging, etc.) may be used during physical therapy or a prehabilitation program to collect information on patient kinematics, alignment, movement, fitness, heart rate, electrocardiogram data, breathing rate, temperature, oxygenation, sleep patterns, activity frequency and intensity, sweat, perspiration, air circulation, stress, step pressure or push-off power, balance, heel strike, gait, fall risk, frailty, overall function, etc. Other types of systems or devices that may be used in the preoperative measurement system 100 may include electromyography or EMG systems or devices, motion capture (mocap) systems, sensors using machine vision (MV) technology, virtual reality (VR) or augmented reality (AR) systems, etc.

### Preoperative Data 1000

The preoperative data 1000 may be data collected, received, and/or stored prior to an initiation of a medical treatment plan or medical procedure. As shown by the arrows in FIG. 2, the preoperative data 1000 may be collected using the preoperative measurement systems 100, from memory system 20 (e.g., cloud storage system) of the image analysis system 10, and from output systems 200 (e.g., from a prior procedure) for one or more continuous feedback loops. Some of the preoperative data 1000 may be directly sensed via one or more devices (e.g., wearable motion sensors or mobile devices) or may be manually entered by a medical professional, patient, or other party. Other preoperative data 1000 may be determined (e.g., by image analysis system 10) based on directly sensed information, input information, and/or stored information from prior medical procedures.

As previously described, the preoperative data 1000 may include imaging data 1010, patient data and/or medical history 1020, information on a planned procedure 1030, surgeon data 1040, and prior procedure data 1050.

The imaging data 1010 may include one or more images (e.g., raw images), videos, or scans of a patient's anatomy collected and/or acquired by the imaging device 110. The image analysis system 10 may receive and analyze one or more of these images to determine further imaging data 1010, which may be used as further input preoperative data 1000. In some examples, imaging device 110 may analyze and/or process the one or more images, and send any analyzed and/or processed imaging data to the image analysis system 10 for further analysis.

The one or more images of the imaging data 1010 may illustrate or indicate, and the image analysis system 10 may be configured to identify and/or recognize, in the images: bone, cartilage, or soft tissue positions or alignment, composition or density, fractures or tears, bone landmarks (e.g., condyle surface, head or epiphysis, neck or metaphysis, body or diaphysis, articular surface, epicondyle, lateral epicondyle, medial epicondyle, process, protuberance, tubercle vs tuberosity, tibial tubercle, trochanter, spine, linea or line, facet, crests and ridges, foramen and fissure, meatus, fossa and fovea, incisure and sulcus, and sinus), geometry (e.g., diameters, slopes, angles) and/or other anatomical geometry data such as deformities or flare (e.g., coronal plane deformity, sagittal plane deformity, lateral femoral metaphyseal flare, or medial femoral metaphyseal flare). Such geometry is not limited to overall geometry and may include relative dimensions (e.g., lengths or thicknesses of a tibia or femur).

The one or more images of the imaging data 1010 may indicate (and/or the image analysis system 10 may determine, based on the one or more received images) morphology and/or anthropometrics (e.g., physical dimensions of internal organs, bones, etc.), fractures, slope (e.g., anterior-posterior (AP) slope or medial-lateral (ML) slope) or angular data, tibial slope, posterior tibial slope or PTS, bone quality and/or density or other measures of bone health (e.g., bone mineral or bone marrow density, bone softness or hardness, or bone impact), etc. Bone density may be determined separately using the image analysis system 10, as described in further detail below, and/or may be collected or supplemented using, for example, indent tests or a microindentation tool. Imaging data 1010 may not be limited to strictly bone data and may be inclusive of other internal imaging data, such as of cartilage, soft tissue, or ligaments.

The imaging data 1010 may indicate or be used to determine, such as via the image analysis system 10, osteophyte size, volume, or positions; bone loss; joint space; B-score; bone quality/density; skin-to-bone ratio; bone loss; hardware detection; anterior-posterior (AP) and medial-lateral (ML) distal femur size, and/or joint angles. Analysis and/or calculations that may be derived from the images or scans will be described in further detail below when describing the image analysis system 10 and the GUIs 250.

Patient data and/or medical history 1020 may include information about the instant patient on identity (e.g., name or birthdate), demographics (e.g., patient age, gender, height, weight, nationality, body mass index (BMI), etc.), lifestyle (e.g., smoking habits, exercise habits, drinking habits, eating habits, fitness, activity level, frequency of climbing activities such as up and down stairs, frequency of sit-to-stand movements or bending movements such as when entering and exiting a vehicle, steps per day, activities of daily living or ADLs performed, etc.), medical history (e.g., allergies, disease progressions, addictions, prior medication use, prior drug use, prior infections, frailties, comorbidities, prior surgeries or treatment, prior injuries, prior pregnancies, utilization of orthotics, braces, prosthetics, or other medical devices, etc.), assessments and/or evaluations (e.g., laboratory tests and/or bloodwork, American Society of Anesthesiology or ASA score and/or fitness for surgery or aesthesia) electromyography data (muscle response or electrical activity in response to a nerve's stimulation), psychosocial information (e.g., perceived pain, stress level, anxiety level, mental health status, PROMS (e.g., knee injury and osteoarthritis outcome score or KOOS, hip disability and osteoarthritis outcome score or HOOS, pain virtual analog scale or VAS, PROMIS Global 10 or PROMIS-10, EQ-5D, a mental component summary, satisfaction or expectation information, etc.), past biometrics (e.g., heart rate or heat rate variability, electrocardiogram data, breathing rate, temperature (e.g., internal or skin temperature), fingerprints, DNA, etc.), past kinematics or alignment data, past imaging data, data from prehabilitation programs or physical therapy (e.g., average load bearing time) etc. Medical history 1020 may include prior clinical or hospital visit information, including encounter types, dates of admission, hospital-reported comorbidity data such as Elixhauser and/or Charlson scores or selected comorbidities (e.g., ICD-10 POA), prior anesthesia taken and/or reactions, etc. This list, however, is not exhaustive, and preoperative data 1000 may include any other patient specific information, clinical information, and/or surgeon or practitioner specific information (e.g., experience level).

Patient data 1020 may come from EMR 120, user interfaces 130, from memory system 20, and/or from robotic platform 140, but aspects disclosed herein are not limited to a collection of the patient data 1020. For example, other types of patient data 1020 or additional data may include data on activity level; kinematics; muscle function or capability; range of motion data; strength measurements and/or force measurements push-off power, force, or acceleration; a power, force, or acceleration at a toe during walking; angular range or axes of joint motion or joint range of motion; flexion or extension data, including step data (e.g., measured by a pedometer), gait data or assessments; fall risk data; balancing data; joint stiffness or laxity data; postural sway data; data from tests conducted in a clinic or remotely; etc.

Information on a planned procedure 1030 may include logistical information about the procedure and substantive information about the procedure. Logistical planned procedure 1030 information may include information about a planned site of the procedure such as a hospital, ambulatory surgery center (ASC), or an operating room; a type of procedure or surgery to be performed (e.g., total or partial knee arthroplasty or replacement, total or partial hip arthroplasty or replacement, spine surgery, patella resurfacing, etc.); scheduling or booking information such as a date or time of the procedure or surgery, planning or setup time, registration time, and/or bone preparation time; a disease or infection state of the surgeon; a name of the primary surgeon or doctor who plans to perform the procedure; equipment or tools required for the procedure; medication or other substances required (e.g., anesthesia type) for the procedure; insurance type or billing information; consent and waiver information; etc. Substantive planned procedure 1030 information may include a surgeon's surgical or other procedure or treatment plan, including planned steps or instructions on incisions, a side of the patient's body to operate on (e.g., left or right) and/or laterality information, bone cuts or resection depths, implant design, type, and/or size, implant alignment, fixation or tool information (e.g., implants, rods, plates, screws, wires, nails, bearings used), cementing versus cementless techniques or implants, final or desired alignment, pose or orientation information (e.g., capture gap values for flexion or extension, gap space or width between two or more bones, joint alignment), planning time, gap balancing time, extended haptic boundary usage, etc. This initial planned procedure 1030 information may be manually prepared or input by a surgeon and/or previously prepared or determined using one or more algorithms.

Surgeon data 1040 may include information about a surgeon or other staff planned to perform the planned procedure 1030. Surgeon data 1040 may include identity (e.g., name), experience level, fitness level, height and/or weight, etc. Surgeon data 1040 may include number of surgeries scheduled for a particular day, number of complicated surgeries scheduled on the day of a planned procedure, average surgery time, etc.

Prior procedure data 1050 may include information about prior procedures performed on a same or prior patient. Such information may include the same type of information as in planned procedure data 1030 (e.g., instructions or steps of a procedure, bone cuts, implant design, implant alignment, etc.) along with outcome and/or result information, which may include both immediate results and long-term results, complications after surgery, length of stay in a hospital, revision surgery data, rehabilitation data, patient motion and/or movement data, etc. Prior procedure data 1050 may include information about prior procedures of prior patients sharing at least one same or similar characteristic (e.g., demographically, biometrically, disease state, etc.) as the instant patient. Prior procedure data 1050 may additionally or alternatively include a predicted procedure generated for a prior patient and an associated confirmed surgery received for the prior patient. Prior procedure data 1050 may include a plurality of predicted surgeries generated for a respective plurality of prior patients and a respective plurality of confirmed surgeries received for the plurality of prior patients. A predicted procedure may be generated by the image analysis system 10, as described in further detail below.

Preoperative data 1000 may include any other additional or supplemental information stored in memory system 20, which may also include known data and/or data from third parties, such as data from the Knee Society Clinical Rating System (KSS) or data from the Western Ontario and McMaster Universities Osteoarthritis Index (WOMAC).

### The Image Analysis System 10

The image analysis system 10 may be an artificial intelligence (AI) and/or machine learning system that is “trained” or that may learn and refine patterns between preoperative data 1000, outputs 2000, and actual results 12 (FIG. 1) to make determinations. The image analysis system 10 may be implemented using one or more computing platforms, such as platforms including one or more computer systems and/or electronic cloud processing systems. Examples of one or more computing platforms may include, but are not limited to, smartphones, wearable devices, tablets, laptop computers, desktop computers, Internet of Things (IoT) device, remote server/cloud based computing devices, or other mobile or stationary devices. The image analysis system 10 may also include one or more hosts or servers connected to a networked environment through wireless or wired connections. Remote platforms may be implemented in or function as base stations (which may also be referred to as Node Bs or evolved Node Bs (eNBs)). Remote platforms may also include web servers, mail servers, application servers, etc.

The image analysis system 10 may include one or more communication modules (e.g., WiFi or Bluetooth modules) configured to communicate with preoperative measurement systems 100, output system 200, and/or other third-party devices, etc. For example, such communication modules may include an Ethernet card and/or port for sending and receiving data via an Ethernet-based communications link or network, or a Wi-Fi transceiver for communication via a wireless communications network. Such communication modules may include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, LTE, 4G, 5G, Bluetooth, near field communication (NFC), radio frequency identifier (RFID), ultrawideband (UWB), etc.). Such communication modules may include a radio interface including filters, converters (for example, digital-to-analog converters and the like), mappers, a Fast Fourier Transform (FFT) module, and the like, to generate symbols for a transmission via one or more downlinks and to receive symbols (e.g., via an uplink).

The image analysis system 10 may further include the memory system 20 and a processing circuit 40. The memory system 20 may have one or more memories or storages configured to store or maintain the preoperative data 1000, outputs 2000, and stored data 30 from prior patients and/or prior procedures. The preoperative data 1000 and outputs 2000 of an instant procedure may also become stored data 50. Although certain information is described in this specification as being preoperative data 1000 or outputs 2000, due to continuous feedback loops of data (which may be anchored by memory system 20), the preoperative data 1000 described herein may alternatively be determinations or outputs 2000, and the determined outputs 2000 described herein may also be used as inputs into the image analysis system 10. For example, some preoperative data 1000 may be directly sensed or otherwise received, and other preoperative data 1000 may be determined, processed, or output based on other preoperative data 1000. Although the memory system 20 is illustrated close to processing circuit 40, memory system 20 may include memories or storages implemented on separate circuits, housings, devices, and/or computing platforms and in communication with image analysis system 10, such as cloud storage systems and other remote electronic storage systems.

The memory system 20 may include one or more external or internal devices (random access memory or RAM, read only memory or ROM, Flash-memory, hard disk storage or HDD, solid state devices or SSD, static storage such as a magnetic or optical disk, other types of non-transitory machine or computer readable media, etc.) configured to store data and/or computer readable code and/or instructions that completes, executes, or facilitates various processes or instructions described herein. The memory system 20 may include volatile memory or non-volatile memory (e.g., semiconductor-based memory device, a magnetic memory device and system, an optical memory device and system, fixed memory, or removable memory). The memory system 20 may include database components, object code components, script components, or any other type of information structure to support the various activities described herein. In some instances, the memory system 20 may be communicably connected to the processing circuit 40 and may include computer code to execute one or more processes described herein. The memory system 20 may contain a variety of modules, each capable of storing data and/or computer code related to specific types of functions.

The processing circuit 40 may include a processor 42 configured to execute or perform one or more algorithms 90 based on received data, which may include the preoperative data 1000 and/or any data in the memory system 20 to determine the outputs 2000. The preoperative data 1000 may be received via manual input, retrieved from the memory system 20, and/or received direction from the preoperative measurement systems 100. The processor 42 may be configured to determine patterns based on the received data.

The processor 42 may be implemented as a general purpose processor or computer, special purpose computer or processor, microprocessor, digital signal processor (DSPs), an application specific integrated circuit (ASIC), one or more field programmable gate arrays (FPGAs), a group of processing components, processor based on a multi-core processor architecture, or other suitable electronic processing components. The processor 42 may be configured to perform machine readable instructions, which may include one or more modules implemented as one or more functional logic, hardware logic, electronic circuitry, software modules, etc. In some cases, the processor 42 may be remote from one or more of the computing platforms comprising the image analysis system 10. The processor 42 may be configured to perform one or more functions associated with the image analysis system 10, such as precoding of antenna gain/phase parameters, encoding and decoding of individual bits forming a communication message, formatting of information, and overall control of one or more computing platforms comprising the image analysis system 10, including processes related to management of communication resources and/or communication modules.

In some aspects, the processing circuit 50 and/or memory system 20 may contain several modules related to medical procedures, such as an input module, an analysis module, and an output module. The image analysis system 10 need not be contained in a single housing. Rather, components of the image analysis system 10 may be located in various different locations or even in a remote location. Components of the image analysis system 10, including components of the processing circuit 40 and the memory system 20, may be located, for example, in components of different computers, robotic systems, devices, etc. used in surgical procedures.

The image analysis system 10 may use the one or more algorithms 90 to make intermediate determinations and to determine the one or more outputs 2000. The one or more algorithms 90 may be configured to determine or glean data from the preoperative data 1000, including the imaging data 1010. For example, the one or more algorithms 90 may be configured for bone recognition, soft tissue recognition, and/or to make determinations related to the intermediate imaging data 1010 previously described. The one or more algorithms 90 may operate simultaneously and/or separately to determine the one or more outputs 2000 and/or display or express the one or more outputs 2000 via GUIs 250.

The one or more algorithms 90 may be algorithms that are trained using, for example, linear regression, random forest regression, CatBoost regression, statistical shape modelling or SSM, etc. The one or more algorithms 90 may be continuously modified and/or refined based on actual outcomes and/or results 12 (FIG. 1). The one or more algorithms 90 may be configured to use segmentation techniques and/or thresholding techniques on received images, videos, and/or scans of the imaging data 1010 to determine the previously described intermediate imaging data 1010 and/or the one or more outputs 2000. For example, the one or more algorithms 90 may be configured to segment an image (e.g., a CT scan), threshold soft tissue, generate one or more plain text (e.g., .txt) comparisons of certain identified bones or tissues (e.g., tibia and femur), and run code to extract values (e.g., PPT or PTT) and populate a database. The one or more algorithms 90 may be configured to automate data extraction and/or collection upon receiving an image from the imaging device 110.

The one or more algorithms 90 may include a joint-space width algorithm 50, an osteophyte detection algorithm 60, a B-score algorithm 70, an alignment/deformity algorithm 80, a predicted procedure algorithm 85, and a bone mineral density (BMD) algorithm 87. Alternatively, one or more of these algorithms may be combined. For example, the joint-space width algorithm 50, the osteophyte detection algorithm 60, the B-score algorithm 70, the alignment/deformity algorithm 80, the predicted procedure algorithm 85, and the BMD algorithm 87 may be combined in a single or master algorithm. Each of the joint-space width algorithm 50, the osteophyte detection algorithm 60, the B-score algorithm 70, the alignment/deformity algorithm 80, the predicted procedure algorithm 85, and the BMD algorithm 87 may be configured to use not only preoperative data 1000 as input but also determinations and/or outputs 2000 from each other. The preoperative data 1000 may be used to create a variety of intelligent models. In some examples, the intelligent models may be statistical models, finite element models, neural networks, and/or predictive artificial intelligence models, such a foundational learning model. Techniques for determining BMD using medical images are described in the article “A Novel Method of Determining Bone Mineral Density from a Pre-surgical CT scans to aid in Surgical Planning” by Niall Maguire, Jared Weir, Sebastian Lustig, and Alan Brett, which is included herein as Appendix A and is incorporated herein by reference in it's entireties.

Each of the one or more algorithms 90 (e.g., the joint-space width algorithm 50, the osteophyte detection algorithm 60, the B-score algorithm 70, the alignment/deformity algorithm 80, the predicted procedure algorithm 85, and the BMD algorithm 87) may be configured to use image processing techniques to recognize or detect bones, tissues, bone landmarks, etc. and calculate or predict dimensions and/or positions thereof based on images acquired by the imaging device 110. The one or more algorithms 90 are not limited to determinations relating to joint-space width, osteophyte volume, B-score, alignment/deformity, and bone mineral density (BMD), and may include and/or be configured to make other procedural determinations, such as those relating to joint laxity or stiffness, discharge time or length of stay time, frailty, fall risk, balancing assessments, patient readiness, etc. The joint-space width algorithm 50, osteophyte detection algorithm 60, B-score algorithm 70, alignment/deformity algorithm 80, predicted procedure algorithm 85, and BMD algorithm 87 will be described in more detail throughout the description.

The one or more algorithms 90 (e.g., the joint-space width algorithm 50, osteophyte detection algorithm 60, B-score algorithm 70, alignment/deformity algorithm 80, predicted procedure algorithm 85, and BMD algorithm 87) may operate simultaneously (or alternatively, at different times throughout the preoperative and intraoperative periods) and exchange inputs and outputs. The one or more algorithms 90 may be configured to determine other scores, values, and/or parameters and are not limited to joint space width, osteophyte volume, B-score, alignment/deformity, and BMD. For example, the one or more algorithms 90 may be configured to determine scores related to bone density/quality (e.g., T-score), joint stiffness or laxity, patient readiness, bone-to-skin ratio, etc.

The one or more outputs 2000 may include a predicted procedure 2005, a predicted procedure time or duration 2010, a procedure plan 2020, an operating room layout 2030, an operating room schedule 2040, assigned or designated staff 2050, recommended surgeon ergonomics 2070, predicted outcomes 2080 of the procedure, and patient anatomy representations 2090, which may include determined and/or enhanced images displayed on the display 210. Each of these outputs 2000 (e.g., predicted procedure 2005, predicted procedure time or duration 2010, a procedure plan 2020, an operating room layout 2030, an operating room schedule 2040, assigned or designated staff 2050, recommended surgeon ergonomics 2070 predicted outcomes 2080 of the procedure, and patient anatomy representations 2090) may be used as input 1000 to determine other outputs 2000. As such, each of these outputs 2000 (e.g., predicted procedure 2005, predicted procedure time or duration 2010, a procedure plan 2020, an operating room layout 2030, an operating room schedule 2040, assigned or designated staff 2050, recommended surgeon ergonomics 2070, predicted outcomes 2080 of the procedure, and patient anatomy representations 2090) may be based in part on a different output 2000 (e.g., predicted procedure 2005, predicted procedure time or duration 2010, a procedure plan 2020, an operating room layout 2030, an operating room schedule 2040, assigned or designated staff 2050, recommended surgeon ergonomics 2070, predicted outcomes 2080 of the procedure, and patient anatomy representations 2090). For example, operating room schedule 2040, assigned or designated staff 2050, and predicted outcomes 2080 may be based in part on predicted procedure 2005 and/or predicted procedure time or duration 2010. As another example, the patient anatomy representations 2090 may be based on predicted outcomes 2080, but aspects disclosed herein are not limited.

A predicted procedure time 2010 may be a total time or duration of a procedure (e.g., as outlined in the procedure plan 2020), and may further include a time or duration of small steps or processes of the procedure. In some examples, the predicted procedure time 2010 may be a predicted time to complete a portion of a procedure. The predicted outcomes 2080 may include a predicted perceived pain level for the patient, a predicted stress level, anxiety level, and/or mental health status of the patient, a predicted cartilage loss, a predicted risk of infection, a rating of a case difficulty, etc. The predicted outcomes 2080 may also include predictions and/or risks if, during the procedure, a time exceeds (or alternatively, is less than) the predicted procedure time 2010 (for example, how a risk of complication and/or a risk of infection may increase based on the procedure taking longer than the predicted procedure time 2010).

The patient anatomy representations 2090 may be determinations or calculations related to the imaging data 1010 and patient anatomy, and may be displayed on the various GUIs 250 described in more detail later. Patient anatomy representations 2090 may include and/or be based on predicted outcomes 2080, such as predicted cartilage loss, joint space width, etc. Patient anatomy representations 2090 may be based on and/or overlaid on images acquired by imaging device 110 and input as imaging data 1010. In some examples, some or all portions of patient anatomy representations 2090 may be based on prior procedure data 1050 and/or simulations.

The outputs 2000 may be output electronically (e.g., on display 210, a mobile device 220, or any other monitors or displays which may be part of procedure systems 240) or printed physically (e.g., on paper, canvas, or film 230 or other materials via a printer). The display 210 may display one or more GUIs 250 to output the outputs 2000. For convenience of description, the GUIs 250 will be described in more detail hereinafter in connection with the one or more algorithms 90 and the outputs 2000 such as the predicted outcomes 2080 and patient anatomy representations 2090.

### GUIs 250, Algorithms 90, and Outputs 2000

As previously explained, the image analysis system 10 may use the one or more algorithms 90 to determine graphical user interfaces (GUIs) 250, which may be displayed on any of the output systems 210. The GUIs 250 may be interactive when implemented on a touch screen. Although various GUIs 250 are described separately herein, the various GUIs 250 may be displayed simultaneously and/or on a same screen of a display 210.

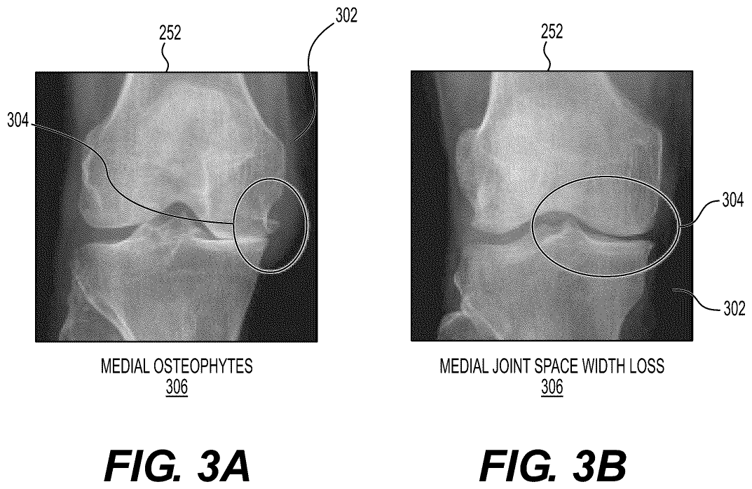

Referring to FIGS. 2 and 3A-3B, the imaging data 1010 may include images 302 acquired of a patient's anatomy, such as CT scans, using the imaging device 110 (e.g., CT scanner 110). These acquired images 302 may illustrate one or more bones and may indicate features of the bones such as osteophytes (or a bone spur that develops on a bone) and, if a joint is illustrated, a joint space width (or a distance between two bones). The one or more algorithms 90 may determine various outputs 2000 by analyzing, using one or more image processing methods, the acquired images 302. The one or more algorithms 90 may also use additional preoperative data 1000 such as patient data 1020 to determine the outputs 2000 to display on the GUIs 250.

The one or more GUIs 250 may include a first or “raw image” GUI 252, which may display one or more acquired images 302. This raw image GUI 252 may, as an example, include visual indicators 304 (e.g., circles, pointers, etc.) which may indicate osteophytes, bone landmarks, or certain joint space widths. A location of the visual indicators 304 may be determined manually (e.g., by a practitioner touching the screen) or by the one or more algorithms 90. In addition, the raw image GUI 252 may display text 306 describing what is being indicated by a visual indicator 304. The example shown in FIG. 3A shows an acquired image 302 of a knee joint. The visual indicator 304 may be a circle encircling one or more osteophytes detected by the one or more algorithms 90 (e.g., osteophyte detection algorithm 60), and the text 360 may display words such as “medial osteophytes” to identify a location of the osteophytes. The example shown in FIG. 3B shows an acquired image 302 of a knee joint. The visual indicator 304 may be a circle encircling a joint space width at a location between a tibia and a femur which is narrower than at other areas or in comparison with other images. The joint space width may be detected and/or calculated by the one or more algorithms 90 (e.g., joint space width volume algorithm 50), and the text 360 may display words such as “medial joint space width loss” to identify a location of the indicated joint space width.

In some examples, the information displayed on each of GUIs 250 may be manipulated by user inputs to operate the GUI 250 and/or procedure system 240. For example, to manipulate the positioning of images displayed on the GUI 250 and/or procedure system 240, a user may make one or more commands (inputs, actuations of one or more buttons, gestures, or other inputs). In some examples, the user may execute one or more commands to perform the steps of the surgical workflow on patient image data, such as a two-dimensional image of the patient's anatomy or a three-dimensional image or model of the patient's anatomy, implant selection, cut selection, and/or other aspects of the surgical workflow, such as manipulating surgical parameters (e.g., position, thickness, type, depth). In some examples, the input/command is a gesture control. A gesture control may be facilitated through machine vision software, which may utilize one or more camera within the procedure system 240. Gesture control may be used for manipulation of a display of a bone(s), an implant, surgical workflow planning, and the like. Some examples of gesture control may be controlled by a user gazing at the display and moving their eyes in a particular way (e.g., eye tracking software), a user moving their hands relative to the display (e.g., executing one or more gestures to actuate one or more commands of procedure system 240), or other types of gesturing movements which may be detected and received by the procedure system 240. In some examples, a user may gesture with their eyes up, down, left, right, and/or blink to interact with the GUI/display/procedure system 240, such as to move an image to the left, right, up or down direction, or rotate an image to the left/right/up/down direction. In some examples, these gestures may be a hand movement up and down, pinching, pulling, swiping, and the like. In one example, moving the hand up and down while pinching may move a display of 3D bone up and down within the GUI. In another example, moving a hand left and right while pinching will rotate a display of 3D bone in the X-axis within a GUI. In a further example, moving a hand while pinching the fingers may move a display of 3D bone in a z-direction and allow the bone to be placed anywhere within a display screen or within a virtual reality environment (e.g. three-dimensional display of virtual model, etc.). In some examples, the gesture control may be dependent on which hand is moving (right or left hand), and which movements each of the right and left hand are performing. In one example, to change the implant angle within a 3D model being displayed, a user may make a first with the left hand, and pinch and move the right hand vertically to change the angle of display of just the 3D model of the implant and not any other 3D models being displayed with the implant with the GUI or other electronic display. In another example, to move the implant, a user may pinch with the right hand and move in the desired direction to adjust the display of the implant in the same manner, e.g. rotate the implant, move the implant within the display, etc.

### 3-D Model and Overall Analysis GUIs

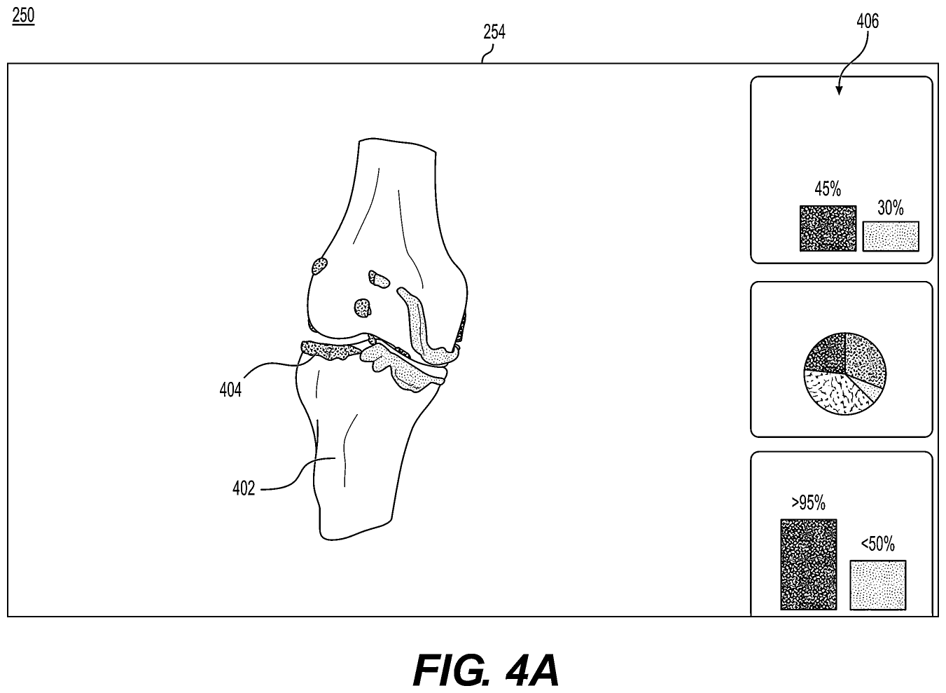

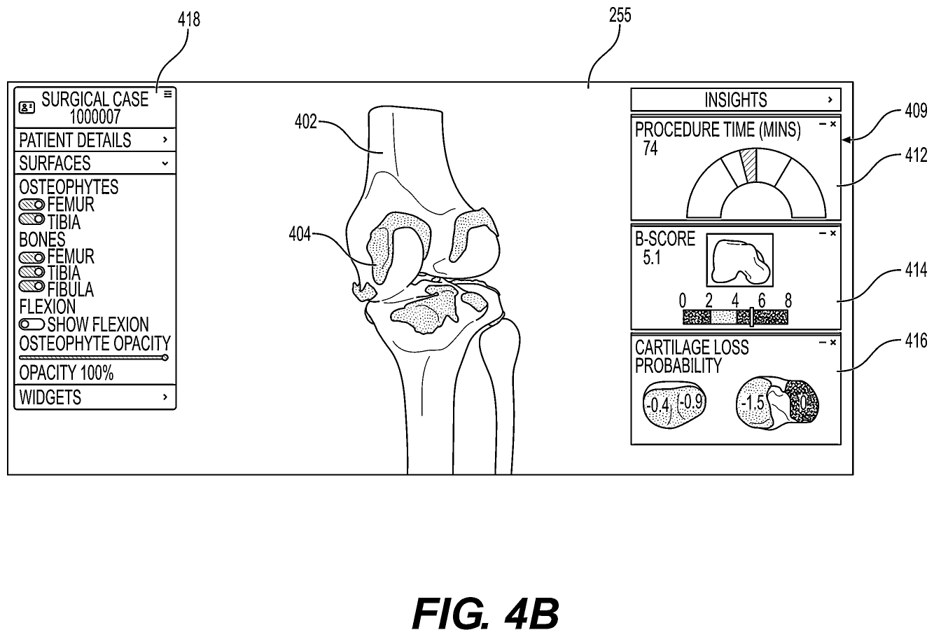

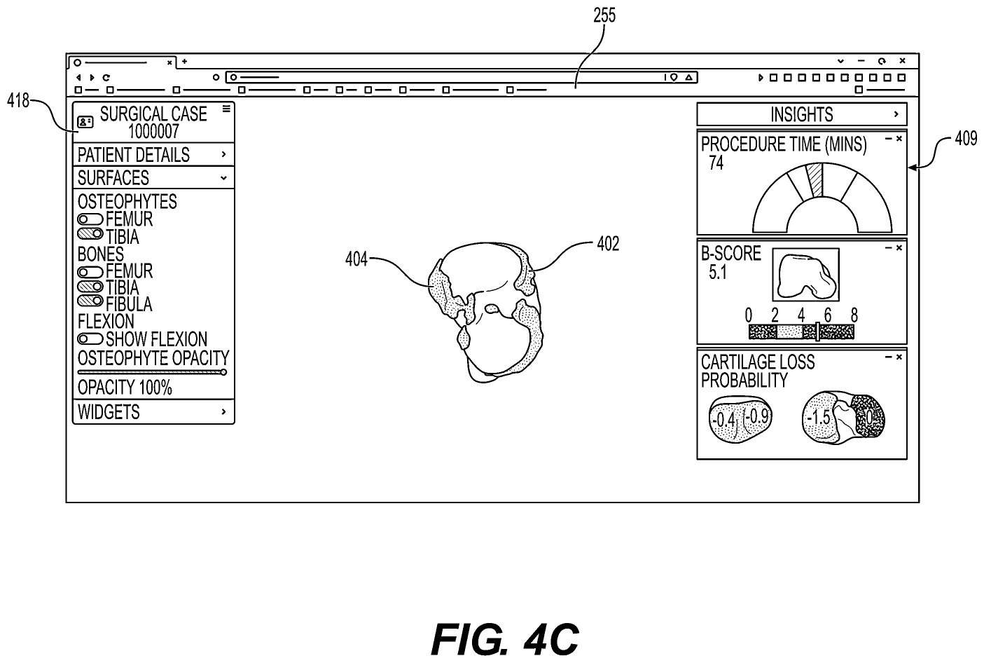

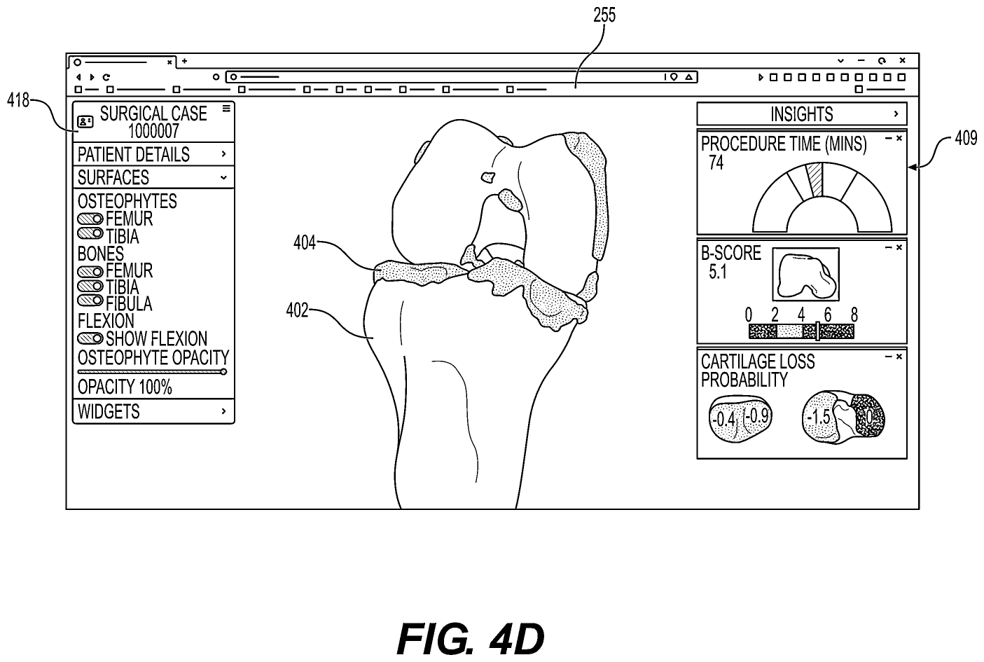

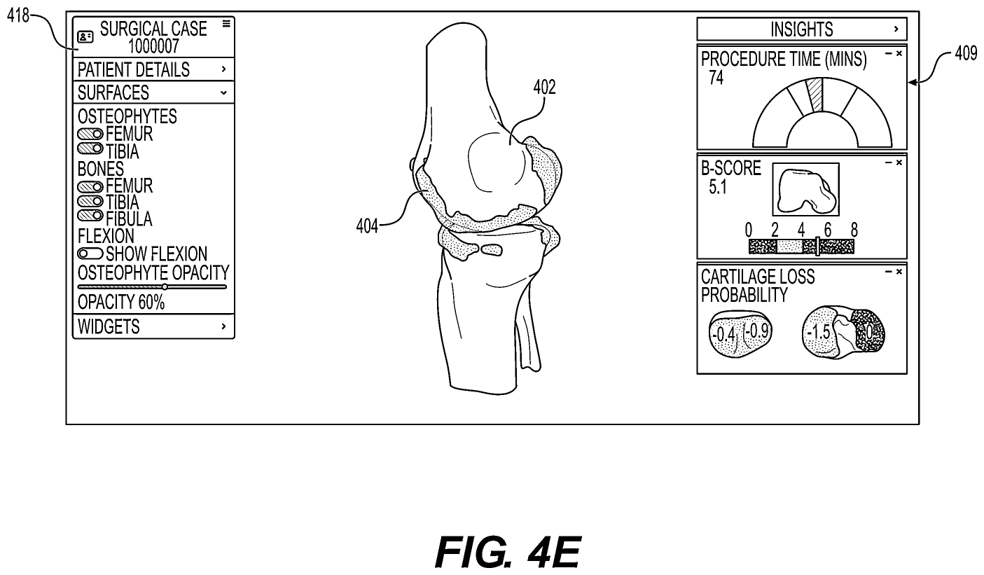

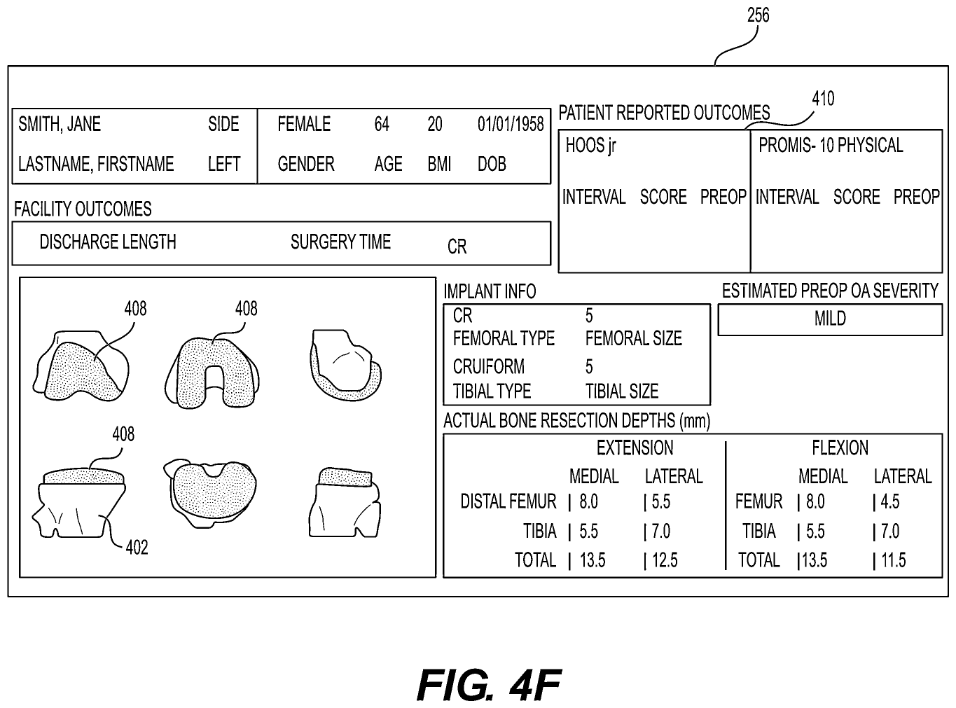

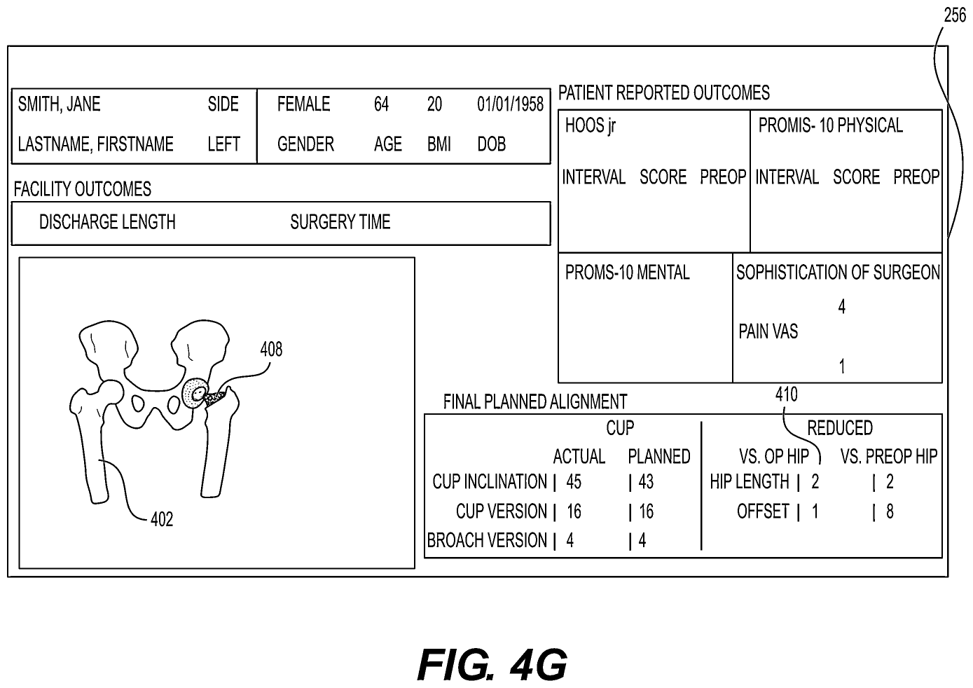

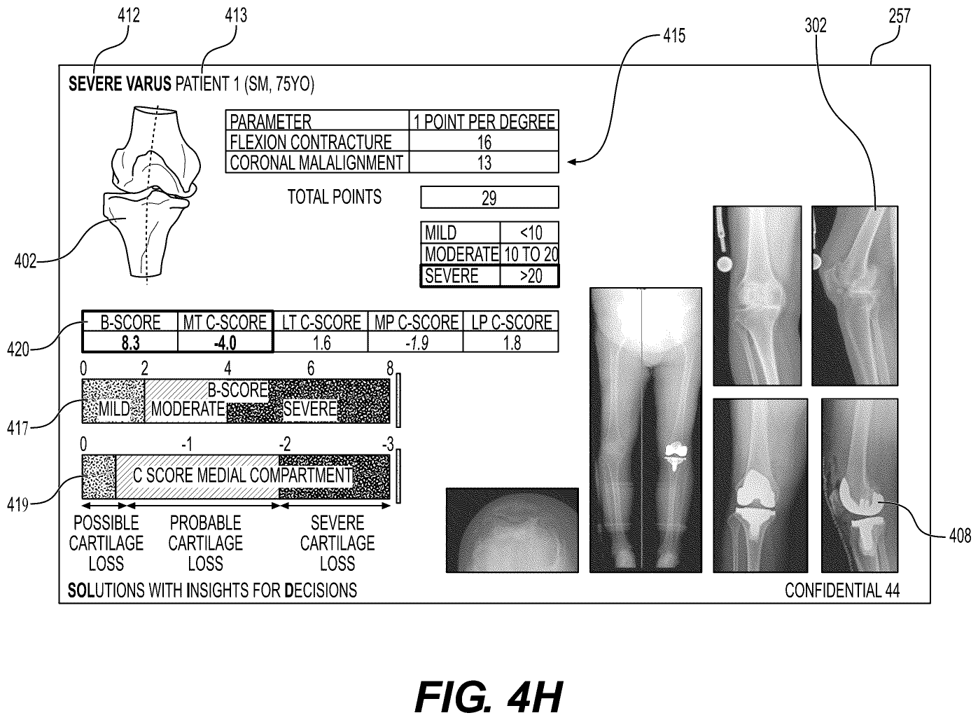

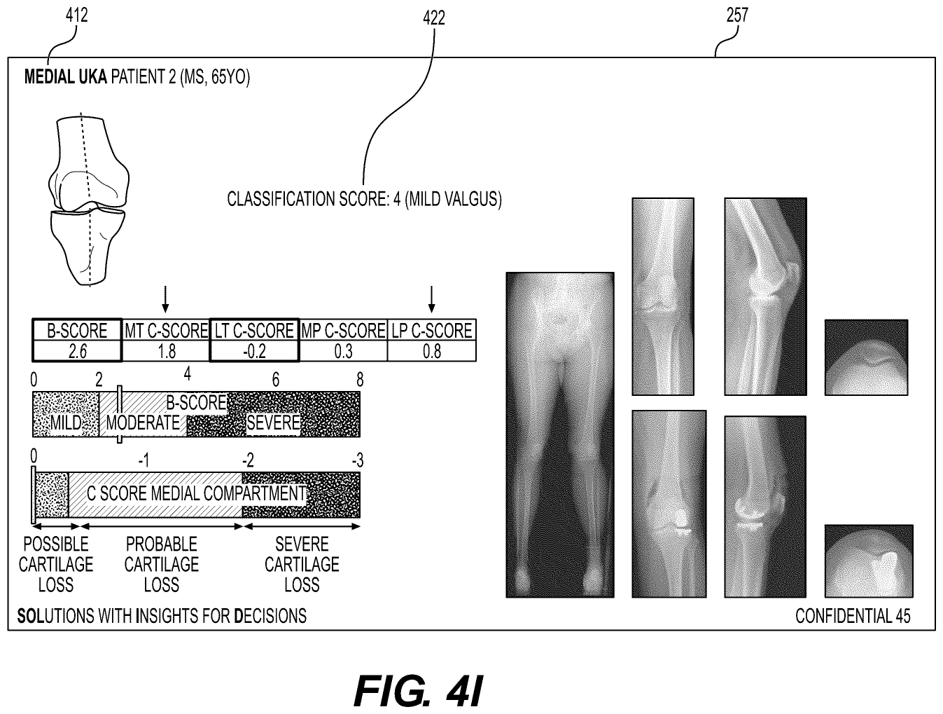

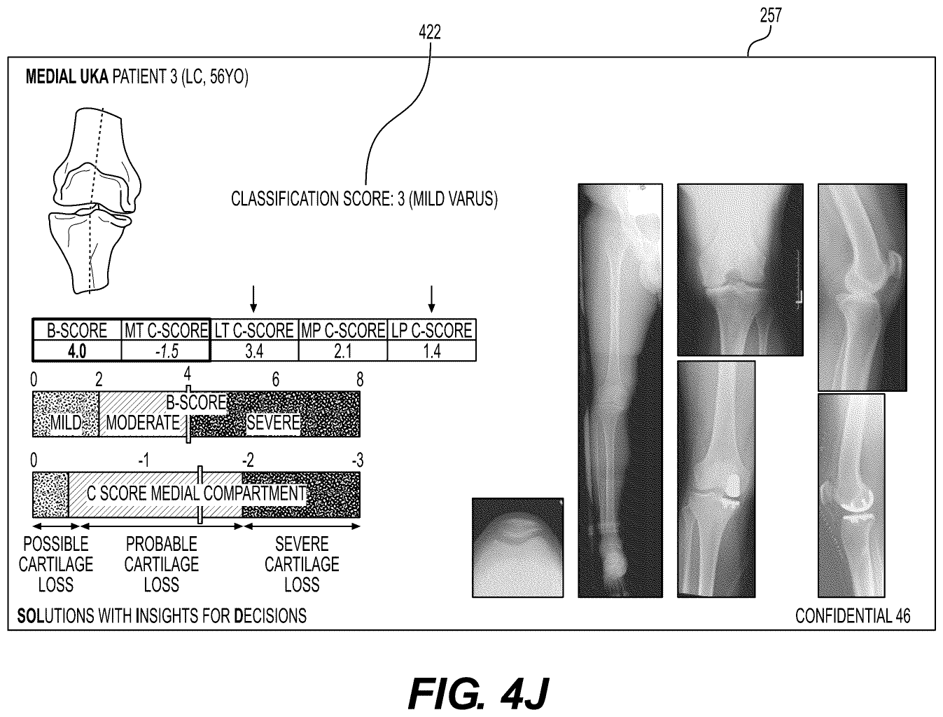

FIGS. 4A through 4J show examples of a second GUI 254, a third GUI 255, a fourth GUI 256, and a fifth GUI 257 showing an overall representation of a patient's anatomy (e.g. second GUI 254) and certain parameters which may be determined by the image analysis system 10 (e.g. fourth GUI 256). The second and third GUIs 254 and 255 may illustrate the patient's anatomy, the fourth GUI 256 may illustrate the patient's anatomy overlaid with one or more implants, and the fifth GUI 257 may display an analysis and/or include acquired images 302. The second and third GUIs 254 and 255 may alternatively be referred to as anatomical or preoperative (preop) GUIs, and the fourth GUI 256 and fifth GUI 257 may alternatively be referred to as a predicted or post-operative (post-op) GUI, though the second and third GUIs 254 and 255 are not limited to illustrating parameters determined preoperatively, and the fourth GUI 256 is not limited to parameters determined postoperatively. The fifth GUI 257 may also be displayed preoperatively, intraoperatively, and/or postoperatively.

### Alignment/Deformity Algorithm 80

Referring to FIGS. 2-4J, the alignment/deformity algorithm 80 and/or the other one or more algorithms 90 may analyze an acquired image 302 to determine outputs 2000 to display on the second GUI 254, third GUI 255, and/or the fourth GUI 256. Alignment and/or deformity may refer to how two or more bones are positioned and/or moved as compared to a healthy patient having a healthy alignment at the two or more bones. The alignment/deformity algorithm 80 may be configured to detect or recognize one or more target bones or joints in the acquired image 302, detect relative positions and/or dimensions of the one or more target bones or joints, detect one or more bone landmarks on the detected bones or joints, and determine or calculate one or more alignment/deformity parameters from the preoperative data 1000 (e.g., imaging data 1010) relating to alignment detection or osteophyte dimensions (e.g., volume) of one or more detected osteophytes in one or more target joints).

The one or more alignment/deformity parameters may include alignment and/or relative position data at certain locations (e.g., joint location), across different directions (e.g., medial or lateral), an average or mean alignment and/or an alignment score, changing or progressing alignment, alignment based on a predicted or determined implant, etc. The alignment/deformity algorithm 80 may assess one or more of these alignment/deformity parameters at one or more bones (e.g., femur or tibia) and/or various anatomical compartments (e.g., anterior lateral, anterior medial, central lateral, central medial, posterior lateral, posterior medial) of one or more bones (e.g., tibia and femur). The alignment/deformity algorithm 80 may also be configured to predict alignment or progression based on other preoperative data 1000, such as kinematics data or activity level data.

The one or more alignment/deformity parameters may include alignment and/or relative positions (e.g., relative to anatomical and/or mechanical axes), such as lower extremity mechanical alignment, lower extremity anatomical alignment, femoral articular surface angle, tibial articular surface angle, mechanical axis alignment strategy, anatomical alignment strategy, natural knee alignment strategy, femoral bowing, varus-valgus deformity and/or angles, tibial bowing, patello-femoral alignment, coronal plane deformity, sagittal plane deformity, extension motion, flexion motion, anterior cruciate ligament (ACL) ligament intact, posterior cruciate ligament (PCL) ligament intact, knee motion and/or range of motion data (e.g., collected with markers appearing in the raw images, videos, or scans) in all three planes during active and passive range of motion in a joint, three dimensional size, quantified data indicating proportions and relationships of joint anatomy in both static and motion, quantified data indicating height of a joint line, metaphyseal flare, medial femoral metaphyseal flare, proximal tibio-fibular joint, coronal tibial diameter, femoral interepicondylar diameter, femoral intermetaphyseal diameter, sagittal tibial diameter, posterior femoral condylar offset-medial and lateral, lateral epicondyle to joint line distance, and/or tibial tubercle to joint line distance. However, aspects disclosed herein are not limited to these alignment parameters.

The one or more alignment/deformity parameters may include data on bone landmarks (e.g., condyle surface, head or epiphysis, neck or metaphysis, body or diaphysis, articular surface, epicondyle, process, protuberance, tubercle vs tuberosity, trochanter, spine, linea or line, facet, crests and ridges, foramen and fissure, meatus, fossa and fovea, incisure and sulcus, and sinus) and/or bone geometry (e.g., diameters, slopes, angles) and other anatomical geometry data. Such geometry is not limited to overall geometry and may include specific lengths or thicknesses (e.g., lengths or thicknesses of a tibia or femur). Imaging data 1010 may also include data on soft tissues for ligament insertions and/or be used to determine ligament insertion sites.

The alignment/deformity algorithm 80 may, based on imaging data 1010 and/or supplemental patient data 1020, determine whether a misalignment, deformity, distances between certain bones, and/or angles between different bones is increasing or decreasing based on a comparison of previously measured alignment/deformity parameters and/or based on a comparison of imaging data from previous image acquisitions. The alignment/deformity algorithm 80 may further determine, predict, or diagnose a disease state or a disease progression (e.g., osteoarthritis or OA) based on the determined alignment/deformity parameters.

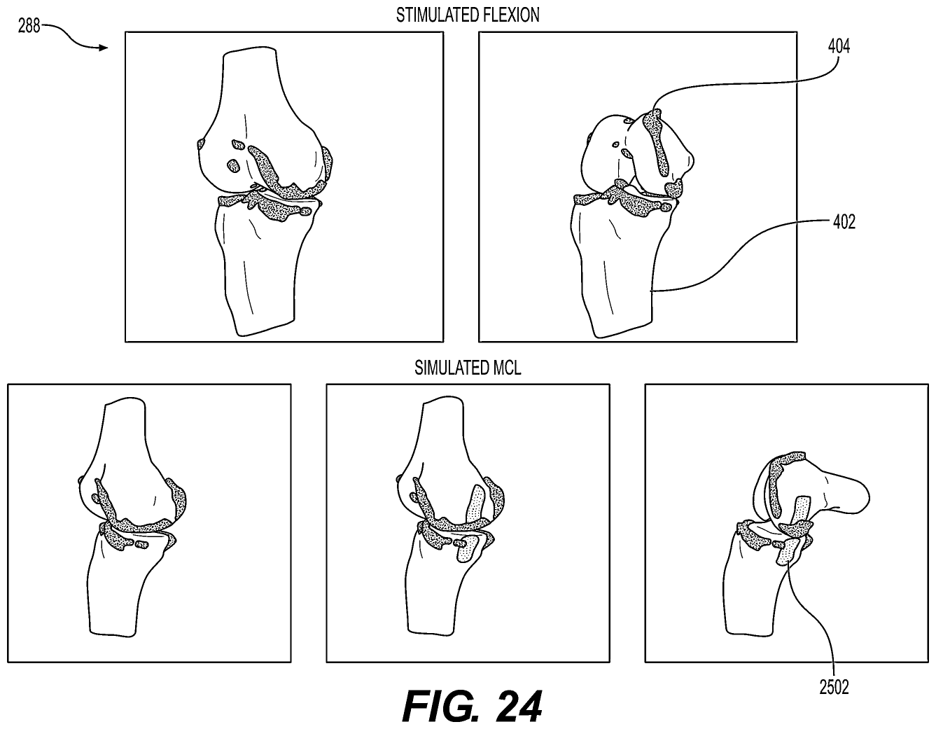

Based on the determined alignment/deformity parameters, the image analysis system 10, using the alignment/deformity algorithm 80 and/or the one or more algorithms 90 collectively, may determine a patient anatomy representation 2090. The determined anatomy representation 2090 may be displayed and/or expressed on one of the GUIs 250 as an artificial or representative model of an instant patient's current anatomy (e.g., bones) such as the representative model 402 in the second GUI 254. In some examples, some or all of the representative models may be simulated and/or based on prior procedure data 1050, such as features that may not be acquired in certain imaging modalities. For example, some X-ray scans may provide more information on bones and cartilage and less on soft tissue, and so a ligament may be simulated in the representative model. As described in more detail with respect to FIG. 24, the one or more algorithms 90 may determine a movement of the representative model, such as a flexion or extension, to illustrate how various portions of anatomy (e.g., the ligament, fibula, etc.) interact with each other or other bones (e.g., tibia and/or femur) during the movement.

Some or all of the artificial model 402 may be based on one or more acquired images 302 that were acquired preoperatively or postoperatively, or even intraoperatively if an imaging device 110 is used during the medical procedure. The one or more algorithms 90 may use previously stored models or standard models of anatomy, which may be included as stored data 30 in memory system 20. The one or more algorithms 90 may detect or recognize bone landmarks, osteophytes, joint space width, and other features in acquired images 302 of an instant patient, and modify the previously stored models to reflect an instant patient's anatomy to determine the artificial model 402. The one or more algorithms 90 may determine colors or other indicators to flag or identify determined features and/or other determinations, such as impingement points. The artificial model 402 may be a three-dimensional representation, and different views may be selected by manipulating the GUI 254 via a touch screen or mouse. For example, the artificial model 402 may show one or more bones, which may be rotated, moved, or spun about various axes (to change the perspective view of the one or more bones) by using a mouse, touch screen, or other user input device.

As exemplified in FIG. 4A, the second GUI 254 may include an artificial model 402 of a knee joint determined by the image analysis system 10 using various knee joint (e.g., tibia, femur, and patella) models stored in the memory system 20, and illustrate a tibia and femur. The relative positions (e.g., joint space width) may be indicative of a determined joint space width, but not necessarily. As exemplified, the second GUI 254 may illustrate osteophytes using colored or shaded indicators 404. The shaded indicators 404 may be toggled on and/or off, which is described and shown in more detail later. Toggling” may refer to displaying or hiding certain features.

The second GUI 254 may also include a plurality of widgets 406 related to determinations or outputs 2000 by the image analysis system 10, such as predicted outcomes 2080. In some examples, the plurality of widgets 406 may be the indication of a statistical ranking of a disease versus normal/healthy anatomy. The plurality of widgets 406 may display the statistical ranking, or other determinations and outputs 2000, as a 3D volumetric measurement, a 2D area or cross section measurement, or a 1D measurement of thickness or direction. For example, the plurality of widgets 406 may include charts, graphs, text, other indicators of a predicted perceived pain the patient may perceive after a medical procedure related to the anatomy depicted in the artificial model 402. The plurality of widgets 406 may also visually indicate other parameters determined by the one or more algorithms 90, such as joint space width, osteophyte volume, B-score, deformities/alignment data, predicted procedure 2005, steps in a procedure plan 2020 (e.g., implant type or design), predicted procedure time 2010, operating room (OR) layout 2030 or operating room (OR) schedule 2040, assigned staff 2050, or surgeon ergonomics 2070. The plurality of widgets 406 may include graphs that compare certain parameters to those of a healthy patient with similar characteristics (e.g., gender, age, medical history) as the instant patient, such as a B-score, joint-space width, or osteophyte volume. The plurality of widgets 406 may be or include selectable icons which, when clicked, present enlarged and/or additional information (e.g., more textual information on perceived pain and recommended steps to reduce patient pain).

As shown in FIGS. 4B through 4E, the primary GUI 255 may be an alternative or in addition to the second GUI 254. The third GUI 255 may include an artificial model 402, one or more indicators 404, a plurality of widgets or cards 409, and a menu 418, which are described in more detail in the explanations of the one or more algorithms 90.

The artificial model 402 may be a simulated model or a model based on a patient's bone(s) (e.g., from acquired images 302). The artificial model 402 may be a model of a joint (e.g., knee joint) determined by the image analysis system 10 using various joint or bone (e.g., tibia, femur, and patella) models stored in the memory system 20 (and/or based on acquired images 302), and illustrate a tibia and femur. The relative positions (e.g., joint space width) may be indicative of a determined joint space width, but not necessarily. The artificial model 402 may depict a preoperative condition of a patient's anatomy, a preoperative prediction of the patient's anatomy after undergoing various treatments (including a prediction of the patient's anatomy after the patient did not undergo treatment), an intraoperative condition and/or prediction based on intraoperative data, and/or a postoperative condition of the patient's anatomy and/or prediction of long-term anatomy or movement based on intraoperative and/or postoperative data, etc.

The indicators 404 may highlight areas of interest, such as osteophytes. As exemplified, the third GUI 255 may illustrate osteophytes using colored or shaded indicators 404. The shaded indicators 404 may be toggled on and/or off, as described in more detail with reference to the menu 418.

The third GUI 255 may also include a plurality of widgets or cards 409 related to determinations or outputs 2000 by the image analysis system 10, such as predicted procedure 2005 or predicted outcomes 2080, and may include charts, graphs, texts, metrics, etc. as described in connection to widgets 406 on the second GUI 254. As an example, the plurality of widgets 409 may include a predicted procedure time widget 412, a B-score widget 414, and/or a C-score or predicted cartilage loss widget 416. A practitioner or user may click on one of the cards or widgets 409 to display a magnified view of the widget 409, a popup, frame, screen, or new GUI based on one or more GUIs 250 described hereinafter.

The predicted procedure time widget 412 may display information related to predicted procedure time or duration 2010. The procedure time widget 412 may display a number of minutes, hours, etc. of a predicted procedure time (e.g., according the procedure plan 2020 and/or planned procedure 1030). The procedure time widget 412 may also display a visual indication of how long the procedure time 2010 is compared to other procedures and/or similar procedures (e.g., an average time for a similar procedure for a patient having similar characteristics). For example, the procedure time widget 412 may include a gradient bar or semicircle or a radial gradient to indicate a severity of the procedure time 2010. A longer procedure time 412 may be visualized by an indicator that is further right on the gradient bar, and/or by a color highlighted on the gradient bar, such as green to indicate that the procedure time is at or below a threshold procedure time (e.g., average procedure time), orange to indicate that the procedure time is within a first time period above the threshold procedure time, and/or red to indicate that the procedure time above the first time period and/or predicted to increase risks and/or complications. A user may click on the predicted procedure time widget 412 to display a magnified view and/or popup of metrics related to the procedure time. For example, scheduling information and/or availability, a case difficulty, recommended staff assignments, surgical tools, etc. may be displayed.

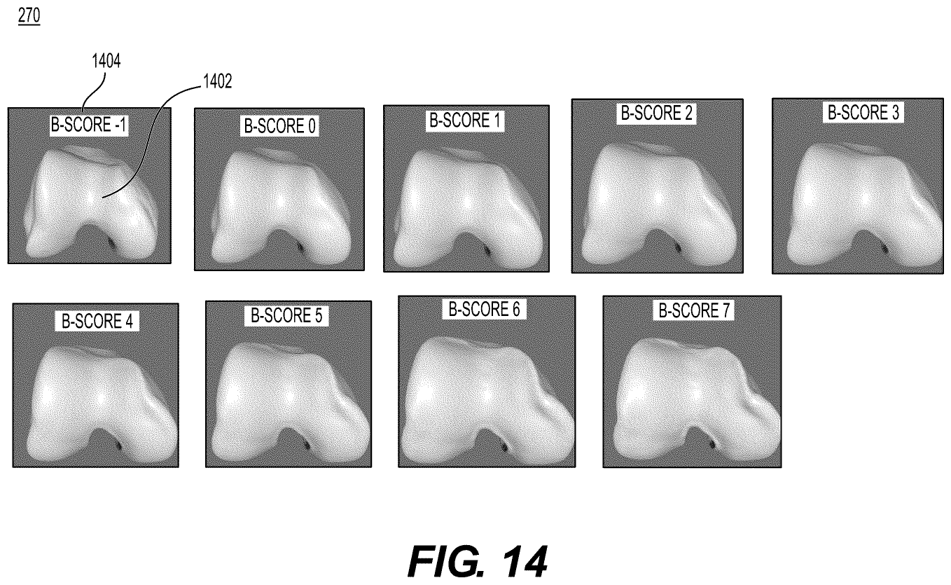

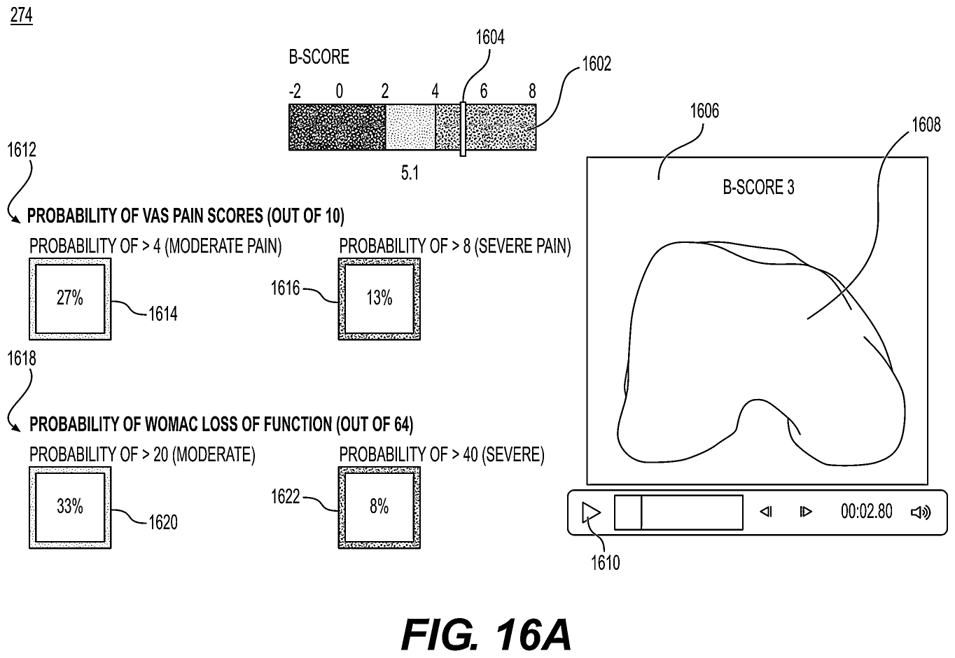

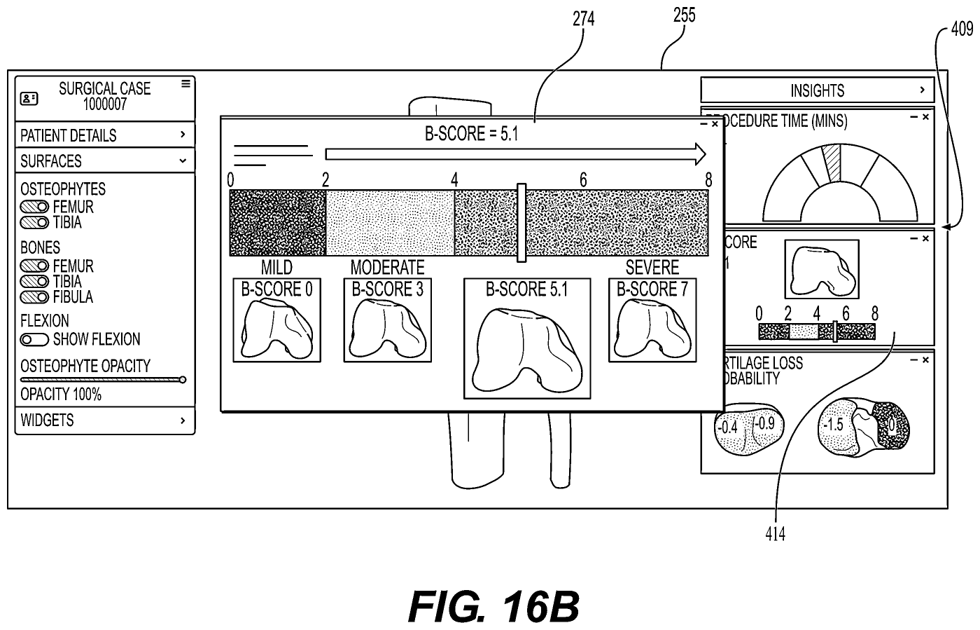

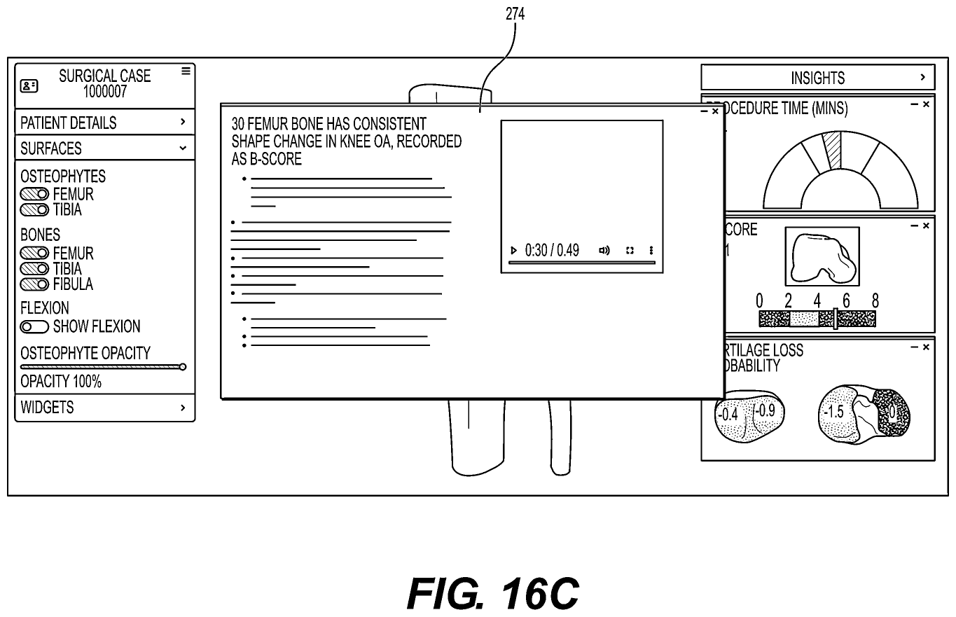

The B-score widget 414 may display information related to B-score, which is described in more detail with reference to B-score algorithm 70. The B-score widget 414 may display a B-score for the patient (e.g., determined by B-score algorithm 70), an image of a bone that represents the B-score (e.g., a femur and/or representative model 1402 shown in FIG. 14), an evolution video or simulation of the bone to represent a changing bone shape (e.g., B-score video 1606 described later with reference to FIG. 16A), and a gradient bar and/or indicator to indicate the B-score (e.g., such as scale 1602 described later with reference to FIGS. 16A through 16C). An indicator on the gradient scale may represent a severity of the B-score and/or a likelihood of complications associated with the B-score. A user may click on the B-score widget 414 to display a magnified view and/or popup of metrics related to the procedure time. For example, B-score video 1606 and/or other B-score GUIs described hereinafter.

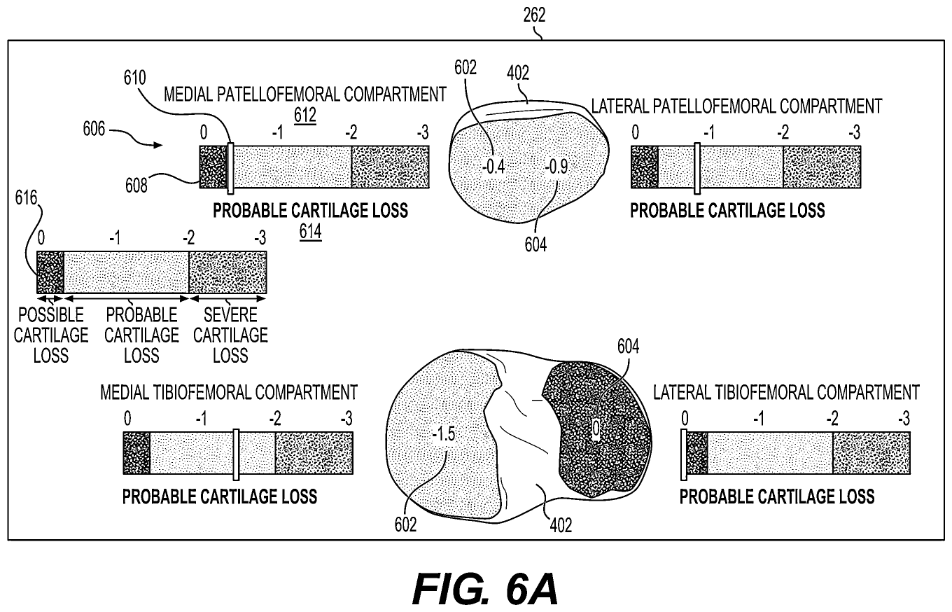

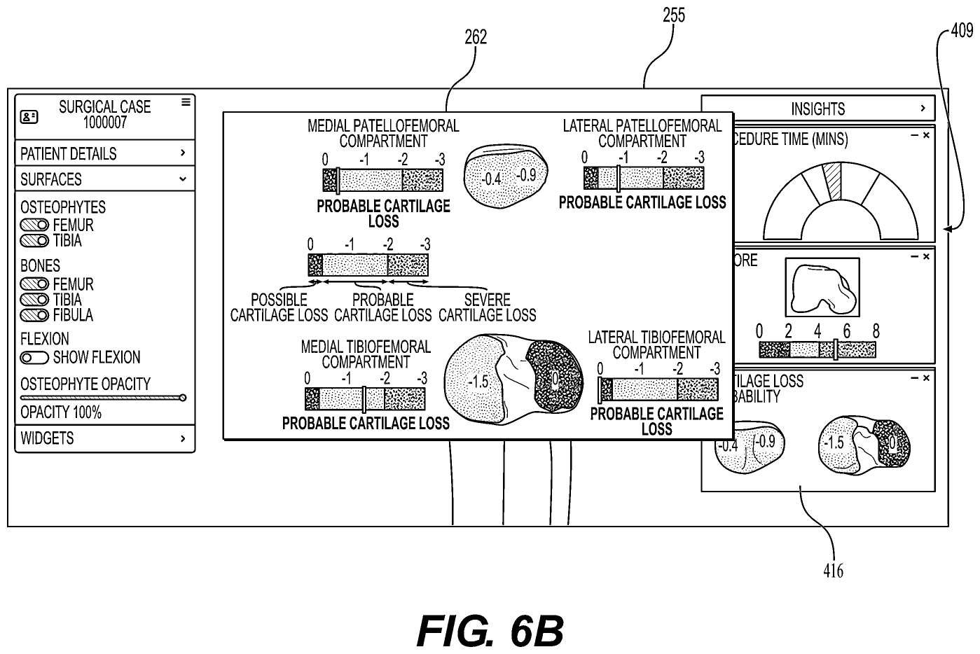

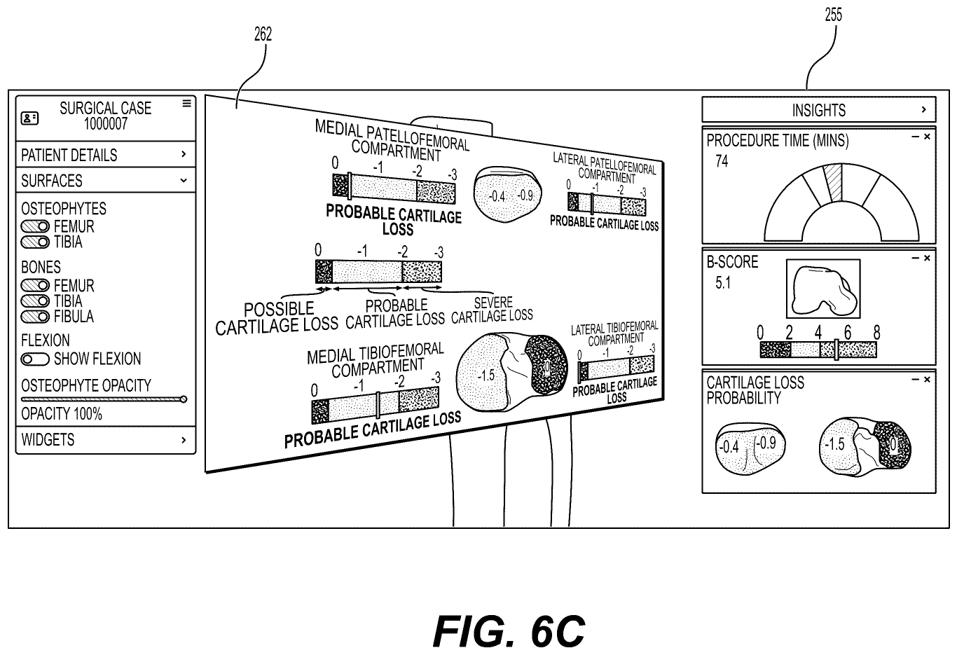

The C-score widget 416 may display information related to a cartilage loss probability and/or a C-score, which is described in more detail with reference to joint-space width algorithm 50. The C-score widget 416 may display a C-score and/or joint-space width parameters or determinations for the patient (e.g., determined by joint-space width algorithm 50), an image of a bone that represents the C-score and/or the joint-space width, and a gradient bar and/or indicator to indicate the C-score (e.g., such as scale 608 described later with reference to FIGS. 6A through 6C). As an example, the C-score widget 416 may display C-score values that correspond to various compartments of the bone, such as values 602 and/or 604 displayed on artificial model 402 and described with reference to FIGS. 6A through 6C. A user may click on the C-score widget 416 to display a magnified view and/or popup of metrics related to joint-space width. For example, gradient 608 described with reference to FIG. 6A and/or other joint-space width GUIs described hereinafter.

Although the plurality of widgets 409 shown in FIG. 4B show a procedure time widget 412, a B-score widget 414, and a C-score widget 416, the plurality of widgets 409 may include alternate or additional widgets, such as widgets displaying scheduling information, surgeon ergonomics, simulated movement, implant design, and/or other widgets depicting outputs 2000 and determinations by the image analysis system 10.

The menu 418 may provide a user interface to allow a user (e.g., practitioner) to change views or orientations, toggle or highlight certain areas, features, or bones, (e.g., hiding or displaying osteophytes on the femur and/or tibia), displaying or hiding certain bones (e.g., tibia or fibular), and/or showing or simulating certain movement (e.g., flexion or extension). The practitioner may also be able to change an opacity of certain highlighted features (e.g., osteophytes) by moving an indicator along a bar to change a level of opacity. The menu 418 may provide various menus and/or submenus, and be provided as a panel or column that is separated or otherwise distinguished from a frame showing the artificial model 402. For example, as shown in FIG. 4B, the menu 418 may be a panel provided on a left side of a screen showing the artificial model 402. In some examples, the menu 418 may be movable. For example, a user may interact with (e.g., click and drag) the menu 418 to change a position of the menu (e.g., so as not to interfere with a display of the artificial model 402). In some examples, a position and/or orientation of the menu 418 may automatically change so as to not interfere with the artificial model 402 (e.g., during a simulated movement).

The menu 418 may display an identification related to a case, such as a case number or other patient or case ID. The menu 418 may include a drop-down menu, button, other user input configured to display information about a patient. For example, as exemplified in FIG. 4B, the menu 418 may include a button for “patient details.” When a user clicks on the button for “patient details,” the third GUI 255 may display a screen, pop-up, or submenu or frame that displays information related to the patient, such as patient data 1020, planned procedure data 1030, and/or such as information shown in FIGS. 4F through 4J. Alternatively or in addition thereto, the menu 418 may include a drop-down menu, button, other user input configured to display information about a surgeon or staffing, case difficulty, procedure, etc. such as surgeon data 1040, assigned staff 2050, OR layout 2030, OR schedule 2040, planned procedure 1030, procedure plan 2020, or any of the preoperative data 1000 and/or outputs 2000.

As previously described, the menu 418 may include various user inputs (e.g., switches, buttons, sliders) to toggle certain features on and/or off. These user inputs to toggle features on and/or off may be provided under a submenu that can be hidden or displayed. For example, as shown in FIG. 4B, the third GUI 255 may display a submenu titled “surfaces.” When the user clicks on this submenu (and/or an arrow displayed on the submenu), the user inputs (e.g., switches) may be shown (e.g., as an extension of menu 418). When the user clicks on the submenu when the user inputs are displayed, the user inputs may be omitted and/or hidden (e.g., the menu 418 may shorten or be reduced in size).

The menu 418 may include switches or other user input (e.g., buttons, sliders, sub-menus or drop-down menus, etc.) that are provided under a section for a feature intended to be toggled on and/or off (e.g., “osteophytes” in FIG. 4B). Under this section, the third GUI 255 may provide a toggle, switch, slider, etc. for each area or bone so that a user may selectively toggle features on/off on that area. For example, under the “osteophytes” section in FIG. 4B, a toggle or switch may be provided for a “femur” and another toggle or switch may be provided for a “tibia.” When a user clicks or slides on the “femur” switch in a first direction, the indicators 404 for the osteophytes on the femur of the artificial model 402 may be displayed, and when the user clicks or slides on the “femur” switch in a second direction, the indicators 404 for the osteophytes on the femur of the artificial model 402 may be omitted or hidden. Similarly, when a user clicks or slides on the “tibia” switch in a first direction, the indicators 404 for the osteophytes on the tibia of the artificial model 402 may be displayed, and when the user clicks or slides on the “tibia” switch in a second direction, the indicators 404 for the osteophytes on the tibia of the artificial model 402 may be omitted or hidden. Although “femur” and “tibia” are used as an example, the menu 418 may refer to different sections and/or different bones and include different labels to toggle on/off osteophytes on those different sections or bones.

The menu 418 may also include switches or other user input (e.g., buttons, sliders, sub-menus or drop-down menus, etc.) that are provided under a section for bones or sections of bones intended to be toggled on and/or off (e.g., “bones” in FIG. 4B). Under this section, the third GUI 255 may provide a toggle, switch, slider, etc. for each section or bone so that a user may selectively toggle on/off that area or bone. For example, under the “bones” section in FIG. 4B, a toggle or switch may be provided for a “femur,” another toggle or switch may be provided for a “tibia,” and another toggle or switch may be provided for a “fibula.” When a user clicks or slides on the “femur” switch in a first direction, the femur of the artificial model 402 may be displayed, and when the user clicks or slides on the “femur” switch in a second direction, the femur of the artificial model 402 may be omitted or hidden. Similarly, when a user clicks or slides on the “tibia” switch in a first direction, the tibia of the artificial model 402 may be displayed, and when the user clicks or slides on the “tibia” switch in a second direction, the tibia of the artificial model 402 may be omitted or hidden. When a user clicks or slides on the “fibula” switch in a first direction, the fibula of the artificial model 402 may be displayed, and when the user clicks or slides on the “fibula” switch in a second direction, the fibula of the artificial model 402 may be omitted or hidden. Although “femur,” “tibia,” and “fibula,” are used as an example, the menu 418 may refer to different sections, bones, tissues, ligaments, etc. and include corresponding labels to toggle on/off those features.

The menu 418 may include switches or other user input (e.g., buttons, sliders, sub-menus or drop-down menus, etc.) that are provided under a section for a displayed movement (e.g., simulated movement) of bones or sections of bones intended to be toggled on and/or off (e.g., “flexion” in FIG. 4B). The third GUI 255 may provide a toggle, switch, slider, etc. for each type of movement (e.g., “flexion,” “extension, etc.) or alternatively for specific positions (e.g., angular positions such as 45 degrees, 90 degrees, etc.) so that a user may selectively display movement of the artificial model 402 to a positional arrangement. For example, under the “flexion” section in FIG. 4B, a toggle or switch may be provided for “show flexion.” As an alternatively, toggles or switches may be provided for certain extents or amounts of flexions (e.g., various angular values or percentages, such as 50% or 100%). When a user clicks or slides on the “show flexion” switch in a first direction, the artificial model 402 may be displayed to undergo flexion, and when the user clicks or slides on the “show flexion” switch in a second direction, a movement of the artificial model 402 may be paused and/or the artificial model 402 may be shown in an opposite (e.g., extension) arrangement.

The menu 418 may include a slide, switches or other user input (e.g., buttons, sub-menus or drop-down menus, etc.) to change an opacity of certain features. For example, FIG. 4B shows a section titled “osteophyte opacity,” a slider bar underneath a label of the section, and an indicator of a percentage of opacity. When the user clicks on the slider bar at a certain position or moves a button along the slider, an opacity of the indicators 404 depicting the osteophytes may change according to the position. For example, a leftmost position of the slider bar may correspond to an opacity of 0%, a rightmost position of the slider bar may correspond to an opacity of 100%, and an extent of the button or position along the slider bar in the right direction may correspond to an opacity percentage. Although “osteophyte opacity” is shown in FIG. 4B, alternatively or in addition thereto, the third GUI 255 may enable changing other opacities, such as an opacity of bones or sections of the artificial model 402 (e.g., to see through to an implant overlaid on the artificial model 402), tissues or ligaments, etc.

The menu 418 may also include a button, switch, etc. to toggle the widgets 409 on and/or off and/or to hide or display a submenu of different widgets 409 to individually toggle the widgets 409 on and/or off. For example, as shown in FIG. 4B, the third GUI 255 may display a submenu titled “widgets.” When the user clicks on this submenu (and/or an arrow displayed on the submenu), all widgets may be displayed 409, and when the user clicks on this submenu again, the widgets may be hidden or omitted. Alternatively, when the user clicks on this submenu, a list of the individual widgets (e.g., procedure time widget 412, B-score widget 414, and/or C-score widget 416) with toggle switches or other user input may be displayed so that the user may selectively display or hide individual widgets 409 independently from each other.

FIG. 4B is an example of a third GUI 255 showing a tibia, femur, and fibula with highlighted or colored osteophytes. FIG. 4C shows the third GUI 255 displaying a top view of the tibia and fibula with the femur toggled off. FIG. 4D shows the third GUI 255 displaying the tibia and femur in flexion. FIG. 4E shows the third GUI 255 displaying the osteophytes with a 60% opacity via indicators 404.

Referring to FIGS. 4F and 4G, the fourth GUI 256 may include an artificial model 402 of an instant patient's current anatomy (e.g., bones), such as the artificial model 402 illustrated in the second GUI 254, overlaid with a planned or recommended implant or prosthetic component 408. The fourth GUI 256 may show a portion of the artificial model 402 (e.g., femur or tibia) and related implants 408 (e.g., femoral implant and tibial implant as part of a knee implant) in various views. The implant 408 may be illustrated in a different color and/or shaded as compared to the artificial model 402. As exemplified in FIG. 4C, the fourth GUI 256 may display a side view, bottom view, and perspective view of a femur 402 overlaid with a femoral implant 408, and a side view, top view, and perspective view of a tibia 402 overlaid with a tibial implant 408. As another example, the fourth GUI 256 may display a pelvic bone, hip bone, iliac crest, and a portion for a femur and other related bones 402 overlaid with one or more hip implants 408 (e.g., femoral head having an acetabular component and/or stem). The implant 408 may be toggled on and/or off based on user implant, and the views of the artificial model 402 (along with the implant 408, if desired) may be manipulated, turned, spun, etc. around a variety of axes by manipulating the fourth GUI 256 with a mouse, touchscreen, or other user input.

The fourth GUI 256 may also include one or more widgets 410. The one or more widgets 410 may include similar widgets and/or information as the widgets 406 of the second GUI 254 and/or the widgets 409 of the third GUI 255, but aspects disclosed herein are not limited. The one or more widgets 410 may include dimensions, alignment, or other geometrical information of the patient's anatomy or the implant 408, or parameters to be used in the procedure plan 2020 to install the implant 408. For example, when used preoperatively or intraoperatively, the one or more widgets 410 may display a recommended thickness, position, type (e.g., stabilizing implant), brand, material, etc. of the implant 408, a recommended bone cut or slope or other preparations to install the implant 408, a number or thickness of shims or augments, etc. The widgets 410 may display alignment and/or deformity information (e.g., as determined by the alignment and/or deformity algorithm 80), patient data 1020 or other inputs 1000 (e.g., range of motion data), etc.

The widgets 410 may display predicted outcomes 2080 and also desired outcomes, and the widget 410 may be interactive such that when a practitioner manipulates certain parameters of the implant 408 (e.g., position, thickness, type), bone cut, etc., which may be done by manipulating the information in the widgets 410 and/or by manipulating the illustrated implant 408 or representative model 402, other predicted outcomes 2080 may change so that the practitioner can assess if at least some of the predicted outcomes 2080 can be more similar to desired outcomes. When used postoperatively, the widgets 410 in the fourth GUI 256 may display actual parameters used during the procedure, and the widgets 410 may also display patient outcomes (which may be reported by the patient or the practitioner, or updated with sensors in the implant 408), predictions further along in recovery, recommendations for revision surgery, etc.

Referring to FIGS. 4H through 4J, the fifth GUI 257 may display a classification or analysis 415 of a patient's condition determined by the one or more algorithms 90 (e.g., alignment/deformity algorithm 80), such as “severe varus,” “mild valgus,” etc. The classification 415 of the patient's condition may be based on a B-score determined by the B-score algorithm 70, a C-score determined by the joint-space width algorithm 50 and/or the one or more algorithms 90, etc.

The fifth GUI 257 may display an artificial model 402, metrics or other measurements 415 relating to alignment or deformity (e.g., as determined by the alignment/deformity algorithm 80), and metrics 420 and/or gradient charts 417 and/or 419 relating to B-score and/or C-core (e.g., determined by the B-score algorithm 70, a C-score determined by the joint-space width algorithm 50 and/or the one or more algorithms 90). For example, the metrics 415 may include a score, points, or positional values (e.g., degrees) corresponding to movement or positional parameters, such as flexion contracture and/or coronal misalignment, and may display a total or sum of the points or values. The metrics 415 may also include a table or scale to help a user assess a severity of the patient's condition based on the total number of points (e.g., mild is less than a first predetermined number of points, such as 10, moderate is between the first predetermined number of points and a second predetermined number of points, such as 20, and severe is greater than the second predetermined number of points). The metrics 420 may include a determined B-score and a determined C-score. With respect to C-score, the metrics 20 may display a C-score for each compartment of a plurality of compartments. For example, the metrics 420 may include a C-score for a medial tibiofemoral (MT) compartment, a lateral tibiofemoral (LT) compartment, a medial patellofemoral (MP) compartment, and/or a lateral patellofemoral (LP) compartment. The gradient charts 417 and/or 419 may include a B-score gradient bar or scale 417 and a C-score gradient bar or scale 419. The B-score gradient bar 417 may be similar to scale 1602 described with reference to FIG. 16A, to visually (e.g., with colors and/or a black and white grayscale and an indicator) depict a severity or value of a determined B-score. Similar to the B-score gradient bar 417, the C-score gradient bar or scale 419 may be similar to scale 608 described with reference to FIG. 6A to visually (e.g., with colors and/or a black and white grayscale and an indicator) depict a severity or value of a determined C-score and/or a predicted cartilage loss. The C-score gradient bar 419 may refer to a total C-score and/or to a C-score of an individual anatomical compartment (e.g., a medial compartment).

The fifth GUI 257 may also display one or more of the acquired images 302, and may further display an implant 408 within the acquired image 302. For example, the fifth GUI 257 may display side and/or lateral views of a patient's anatomy (e.g., left and right side), frontal and/or rear views, top and/or bottom views, etc. both with and without an implant 408. The implant 408 may be a predicted implant model or simulation overlayed on the acquired image 302, or the acquired image 302 may be a postoperative image showing an installed implant 408. In FIGS. 4I and 4J, the fifth GUI 257 may display, in addition or alternatively to metrics 414, a classification score 422 determined by the one or more algorithms 90 to classify or describe the patient's condition.

Joint Space Width, Cartilage Loss, and/or C-Score GUIs

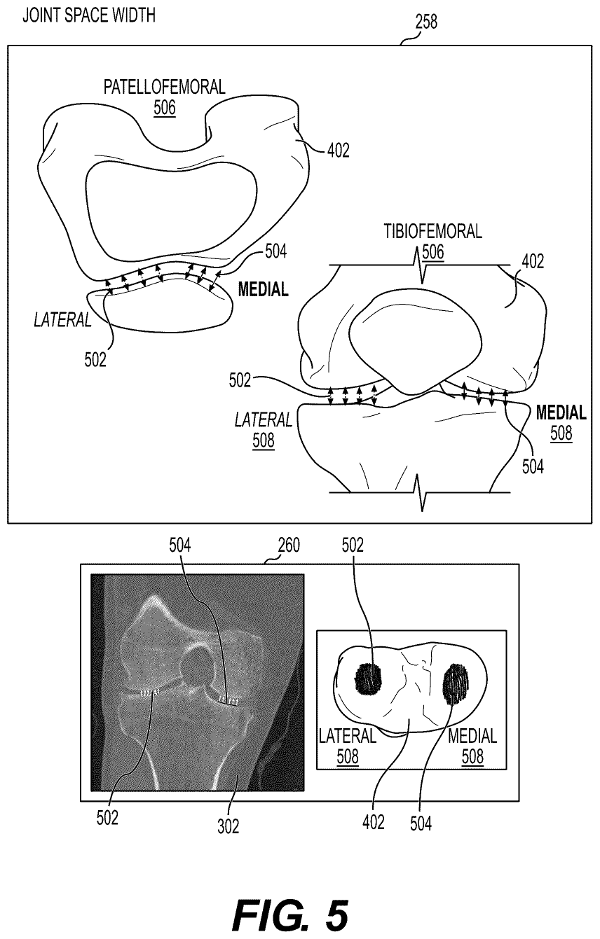

Referring to FIGS. 2 and 5, the image analysis system 10 may determine a joint-space width and related parameters between two or more bones of a joint, and determine or generate one more GUIs 250 to display the joint space width and related parameters.

A joint space width (JSW) may be a distance between two or more bones at a joint. The joint-space width algorithm 50 may be configured to determine one or more JSW parameters from images in the imaging data 1010. The JSW parameters may relate to a joint space width in one or more target joints. The one or more JSW parameters may include joint space widths at predetermined locations, joint space widths across different directions (e.g., medial JSW or lateral JSW), average or mean joint space width (e.g., mean three-dimensional or 3D joint space width), changing joint-space (e.g., joint space narrowing), an average or mean joint space narrowing (e.g., mean 3D joint space narrowing), impingement data, impingement angles, impingement data based on a predicted or determined implant, etc. The joint-space width algorithm 50 may detect and/or reference a plurality (e.g., hundreds) of bone landmarks to determine joint space widths at various positions.

The joint-space width algorithm 50 may assess one or more of these JSW parameters at various anatomical compartments (e.g., anterior lateral, anterior medial, central lateral, central medial, posterior lateral, posterior medial, or, for a knee joint, medial tibiofemoral (MT), lateral tibiofemoral (LT), medial patellofemoral (MP), and/or a lateral patellofemoral (LP)) of one or more bones (e.g., tibia and femur). For example, the joint space width algorithm 60 may determine four JSW parameters (e.g., joint space width in four compartments) in a knee joint. The joint space width algorithm 50 may also be configured to predict joint spaces based on loadbearing and/or unloaded conditions using other preoperative data 1000, such as kinematics data or activity level data. For example, FIG. 5 shows in, GUI 258, a joint space width in a medial tibiofemoral (MT) compartment, a lateral tibiofemoral (LT) compartment, medial patellofemoral (MP) compartment, and a lateral patellofemoral (LP) compartment. FIGS. 6A-6B show measurements for each of the MT, LT, MP, and LP compartments.

The joint space width algorithm 50 may, based on supplemental patient data 1030, determine whether a joint space width is decreasing or narrowing (and/or increasing or widening) based on a comparison of previously measured joint space widths and/or based on a comparison of imaging data from previous image acquisitions. The joint space width algorithm 50 may also determine, estimate, or predict one or more cartilage parameters, such as cartilage thickness or a probability of cartilage loss during the procedure (e.g., by using a Z-score or other statistical measure). This determined cartilage parameter may be based on a determined joint space width or other JSW parameters determined by the joint space width algorithm 50. The predicted cartilage loss may be for each compartment or for the bone.

For example, the joint space width algorithm 50 may determine a mean three-dimensional joint space narrowing (3DJSN) in medial and lateral compartments of a bone such as a tibia and/or a femur. The joint space width algorithm 50 may determine mean 3D joint space width (3DJSW) centrally in each compartment. For each compartment, the joint space width algorithm 50 may compare parameters to those of a healthy patient having similar characteristics as the instant patient, and the image analysis system 10 may use the determinations from the joint space width algorithm 50 along with other preoperative data 1000 or determinations by the other one or more algorithms 90 to determine a disease state or other outputs 2000.

The image analysis system 10 may use JSW parameters determined by the joint space width algorithm 50 to determine, estimate, or predict cartilage loss (e.g., an amount or a probability of cartilage loss). The joint space width algorithm 50 may also be used to determine scores or values in a plurality (e.g., four) of anatomical compartments (e.g., a knee joint) based on joint-space width or cartilage loss, and determine a composite score or C-score based on the determined scores of each of the compartments. The scores for each compartment and/or the C-score may also be based in part on patient data 1020, such as gender, as males and females on average have different cartilage widths. The joint space width algorithm 50 may alternatively be referred to as a C-score algorithm 50. The C-score may correlate to or be proportional to a predicted cartilage loss, such that a higher C-score may indicate a higher probability of cartilage loss and/or a higher severity or amount of predicted cartilage loss.

The joint space width algorithm 50 may determine or select a compartment among the plurality of compartments that should be resurfaced during the procedure, and determine that the procedure plan 2020 should include one or more steps directed to resurfacing the selected compartment. The joint space width algorithm 50 may determine cartilage thickness or loss based on a determined C-score, and may consider patient data 1020 (e.g., gender). The joint space width algorithm 50 may convert a joint-space width (e.g., in mm) to a Z-score or other score. A Z-score may describe a relationship between a particular value (e.g., joint-space width) with a mean or average of a group of values. For example, a Z-score may be measured in terms of standard deviations from the mean such that Z-score of 0 may indicate a value that is identical to the mean score. In some examples, the joint space width algorithm 50 may determine patient data 1020, such as gender, based on the determined JSW parameters (e.g., C-score or Z-score). In some examples, the joint-space width algorithm 50 may determine whether the procedure plan 2020 should include a total or partial arthroplasty (e.g., a total or partial knee arthroplasty).

Based on the determined JSW parameters, the joint-space width algorithm 50 and/or the one or more algorithms 90 collectively may be used to determine one or more of the outputs 2000. In some examples, the joint-space width algorithm 50 may determine and/or predict (or be used to determine and/or predict) a procedure time or duration 2010 to execute a procedure plan 2020. For example, the joint-space width algorithm 50 may determine that a joint space width of a patient is outside of a predetermined range, is narrowing over time and/or is smaller than a first predetermined threshold, or is widening over time and/or is greater than a second predetermined threshold. The image analysis system 10 may, based at least in part on these determinations by the JSW algorithm 50, predict a longer or shorter procedure time 2010, a recommended implant to use in the procedure plan 2020, predicted outcomes 2080 such as cartilage loss, and patient anatomy representations 2090. Other factors (e.g., from patient data 1020) may change the analysis and/or relationship such that the image analysis system 10 and/or the osteophyte joint-space width algorithm 50 may determine certain relationships between higher or lower JSW parameters combined with certain patient data 1020. In addition, the image analysis system 10 may learn other relationships between JSW parameters and predicted outcomes 2080 other than cartilage loss, for example by analyzing prior JSW parameters from prior procedure data 1050.

The GUIs 250 may include a sixth GUI 258 and a seventh GUI 260, which may display JSW parameters determined by the joint space width algorithm 50 in relation to the artificial model 402 (as in sixth GUI 258) and/or in relation to an acquired image 302 (as in seventh GUI 260).

The sixth GUI 258 may display one or more views of an artificial model 402 of a joint (e.g., a knee joint) in a way that illustrates a space between one or more bones of the joint. The sixth GUI 258 may depict a joint space width determined by the joint space width algorithm 50 using JSW lines, arrows, or other symbols 502, 504 that extend between the one or more bones in the joint space width. The JSW lines 502, 504 may be color coded according, for example, a compartment or side of the bone or a direction to which they relate.

As exemplified in FIG. 5, the sixth GUI 258 may display an artificial model 402 of a knee joint, which may include one or more views of a patellofemoral joint and a tibiofemoral joint. As with the other GUIs 250, each of these displayed joints (e.g., patellofemoral joint and tibiofemoral joint) may be manipulated to turn, spin, or rotate around various axes via user input to change views. The sixth GUI 258 may include a first set of JSW lines 502 showing a joint space width at a first (e.g., lateral) side for each of the patellofemoral and tibiofemoral joints, and may further include a second set of JSW lines 504 showing a joint space width at a second (e.g., medial) side for each of the patellofemoral and tibiofemoral joints. The sixth GUI 258 may include joint titles or labels 506 for each joint (patellofemoral and tibiofemoral joints) and side or compartment labels 508 indicating which displayed side or compartment the JSW lines 502, 504 correspond to. As exemplified in FIG. 5, the side labels 508 may indicate which displayed side is a lateral side and which side is a medial side. Although lateral and medial are used in connection with the example of FIG. 5, anterior and posterior labels may be used instead. Aspects disclosed herein are not limited to the information supplied in the labels 506, 508. The lateral JSW lines 502 may be illustrated in a different color than the medial JSW lines 504 for clarity.

The seventh GUI 260 may show similar information as the sixth GUI 258, but may overlay JSW lines 502, 504 on the acquired image 302 instead of or in addition to the artificial model 402. As exemplified in the seventh GUI 260, the seventh GUI 260 may display an acquired image 302 of a knee joint, including a femur and a tibia of the instant patient. The seventh GUI 260 may overlay the JSW lines 502, 504. JSW lines 502 in one area or compartment (e.g., lateral) may appear as a different color than JSW lines 504 in another area or compartment (e.g., medial). In addition, a density of the JSW lines 504 may be proportional to a determined joint space width. The seventh GUI 260 may also show, on a same screen or separate screen, a view of a corresponding artificial model 402 generated from the acquired image 302. As exemplified in FIG. 5, the seventh GUI 260 may show a top view of a portion of the artificial model (e.g., tibia) and color-coded lateral JSW lines 502 and medial JSW lines 504, but aspects disclosed herein are not limited. The artificial model 402 may be spun, turned, rotated, etc.

Although not shown, the sixth GUI 258 and seventh GUI 260 may include widgets, tables, charts, or other information that may indicate (e.g., numerically) the JSW parameters determined by the joint space width algorithm 50, such as the C-score or Z-score. The sixth GUI 258 and seventh GUI 260 may indicate accurate or instant parameters (e.g., of the instant patient's actual bone geometry) and/or may indicate predicted parameters or recovery (e.g., a joint space width after installation of an implant or further down recovery). The sixth GUI 258 and seventh GUI 260 may be used preoperatively, intraoperatively, or postoperatively. Alternatively or in addition thereto, the sixth GUI 258 and the seventh GUI 260 may be implemented as the widgets 406, 409 and/or 410 described with reference to FIGS. 4A through 4E.

Referring to FIGS. 2 and 6A through 6C, the GUIs may include an eighth GUI 262, which may indicate predicted outcomes 2080 related to joint space width. In the example shown in FIG. 6A, the eighth GUI 262 indicates predicted cartilage loss determined by the joint space width algorithm 50. For convenience of description, predicted cartilage loss will be described as an exemplary parameter, but aspects disclosed herein are not limited, as the image analysis system 10 may determine new or different outputs 2000 and/or predicted outcomes 2080 that are based on joint space width.

The eighth GUI 262 may show a view (e.g., top view) of an artificial model 402 of two or more bones of a joint, such as the tibia and the femur. One or more values 602, 604 determined by the joint space width algorithm 50 may be overlaid in the top views of the artificial models 402. The one or more values 602, 604 may include a first value 602 corresponding to a first compartment or side (e.g., medial) and a second value 604 corresponding to a second compartment or side (e.g., lateral). These values 602, 604 may indicate a joint space width (e.g., mm), a score (e.g., C-score or Z-score), or a number or score corresponding to a predicted amount of cartilage loss or a prediction or percentage that cartilage loss will occur. The artificial model 402 may be colorized in a way that corresponds to the values 602, 604.

The eighth GUI 262 may include a cartilage loss display 606 corresponding to each value 602, 604 for each of the displayed artificial models 402 of the joints. The cartilage loss display 606 may include a scale or axis 608. The scale 608 may be a gradient bar that is color coded so that numbers or values indicating healthy cartilage loss (or, as another example, a low likelihood of cartilage loss) appear in green, values indicating extensive, severe, or unhealthy cartilage loss (or, as another example, a high likelihood of cartilage loss) appear in red, and intermediate values appear in yellow or orange. The scale or axis 608 may have periodic numerical indicators. The cartilage loss display 606 may include an indicator (e.g., line) 610 appearing on the scale 608 at a position that corresponds to the value 602, 604. The cartilage loss display 606 may display predicted cartilage loss in each compartment of the bone (e.g., four compartments). The cartilage loss display 606 may include a compartment label 610 indicating a compartment or position corresponding to the value 602, 604 (e.g., medial patellofemoral, lateral patellofemoral, medial tibiofemoral, or lateral tibiofemoral). The cartilage loss display 606 may include a parameter label 614 indicating the displayed parameter (e.g., probable cartilage loss), and may include a key 616 indicating the significance of colors or numbers appearing in the scale 608.

Referring to FIG. 6B, the eighth GUI 262 may be implemented as one of the cards or widgets 409 displayed on third GUI 255. For example, the plurality of widgets 409 may include the cartilage loss or C-score card 416 described with reference to FIG. 4B. A user may click the cartilage loss card 416 to display the eighth GUI 262. Alternatively or in addition thereto, clicking the C-score card 416 may bring up an abbreviated version of eighth GUI 262 that displays some, but perhaps not all, features of eighth GUI 262. FIG. 6B shows an example where the eighth GUI 262 may appear as a popup, a magnified frame of the eighth GUI 262 and/or the C-score card 416, or as a separate frame of the third GUI 255, but aspects disclosed herein are not limited. For example, clicking the cartilage loss card 416 may bring up the eighth GUI 262 as a full screen GUI, as shown in FIG. 6A. Referring to FIG. 6C, the eighth GUI 262 may, upon clicking, rotate or flip to display a “back” of the cartilage loss card 416, which may display textual information videos, etc. or other additional information or analysis (e.g., determinations by the one or more algorithms 90) regarding cartilage loss, C-score, joint-space width, etc., and/or gradient bar 608 or other display features described herein relating to C-score and/or joint-space width.

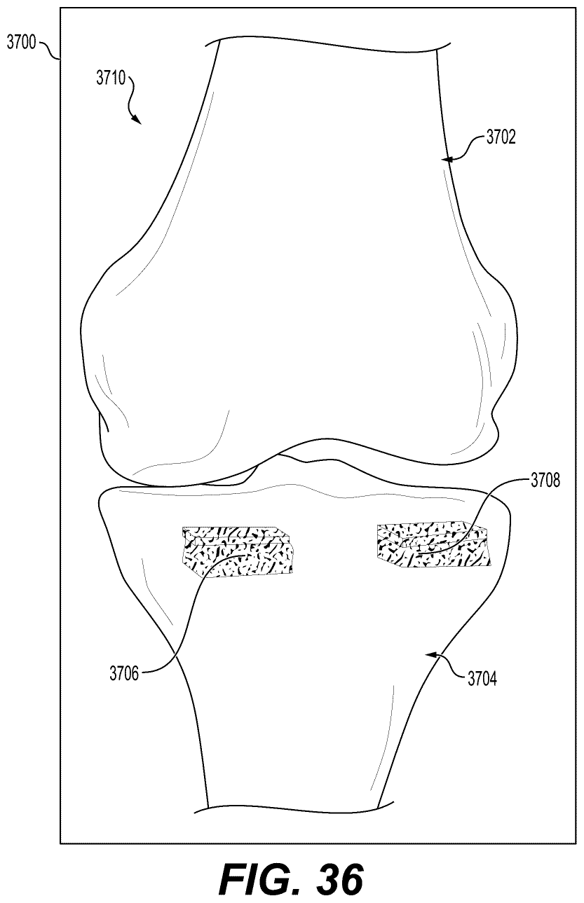

FIG. 36 illustrates another example of a GUI 3600 displaying potential cartilage loss or C-score, as described above. In this example, on the left side of the GUI 3600, the femur 3602 and the tibia 3604 are shown with C-scores overlaid at their respective locations 3606, 3608, 3610, 3612 on femur 3602 and tibia 3604, respectively, to indicate the loss of cartilage at a specific region for a particular surgical plan. Each location corresponds to a compartment such as the medial patellofemoral compartment 3606, lateral patellofemoral compartment 3608, medial tibiofemoral compartment 3610, and lateral tibiofemoral compartment 3612. The GUI 3600 indicates predicted cartilage loss, which may be determined by the joint space width algorithm 50. The predicted cartilage loss and C-score may include a visual indicator to indicate the possibility of cartilage loss. For example, the left side (facing the page) of the GUI 3600 includes the C-score at each location 3606, 3608, 3610, 3612, however, other visual indicators are contemplated. In some examples, each compartment 3606, 3608, 3610, 3612 may include a visual indicator indicative of the possibility of cartilage loss in addition to the numeric C-score, such as a color gradient or pattern. In one example, the visual indicator may be plurality of colors, such as green, yellow, and red, with green being the lowest possibility of cartilage loss and red indicating the highest possibility of cartilage loss. It is further contemplated that additional information relating to alignment and cartilage loss as determined by the joint space width algorithm 50 may displayed with the C-score on GUI 3600. In some examples, the GUI 3600 may have multiple displays for different regions of a single bone 3602, 3604, which may be displayed within a single electronic screen, multiple electronic displays, and/or other display methods known in the art such as virtual reality display or other display devices.

The GUI 3600 may include multiple displays and images for each region of a single bone 3602, 3604. In some examples, the GUI 3600 may display a 2D image of a defined view plane or cross section of a bone 3602, 3604. In another examples, the GUI 3600 may display a 3D model that is repositionable by the used for preferred viewing.

In some examples, such as shown in FIG. 36, additional information relating to the C-score and possibility of cartilage loss may displayed. The right side (facing the page) of the GUI 3600 illustrates indicators 3614, 3616, 3618, 3620 corresponding to each of the medial patellofemoral compartment 3606, lateral patellofemoral compartment 3608, medial tibiofemoral compartment 3610, and lateral tibiofemoral compartment 3612, respectively. In this example illustrated in FIG. 36, the indicator bar 3614 for medial patellofemoral compartment 3606 shows a possible cartilage loss, as indicated by the score in compartment 3606 on the femur 3602, whereas the indicator 3616 for lateral patellofemoral compartment 3608 has a higher score and is indicated as having a severe cartilage loss on the indicator 3616. Similarly, on the tibia 3604, both the medial tibiofemoral compartment 3610, and lateral tibiofemoral compartment 3612 have a high cartilage loss score as illustrated by indicators 3618, 3620. In this example, the indicators 3614, 3616, 3618, 3620 are illustrated as a gradient bar with discrete locations corresponding to a range of potential cartilage loss (e.g., with colors and/or a black and white grayscale and an indicator) depict a severity or value of a determined C-score and/or a predicted cartilage loss. In other examples, other graphical indicators are contemplated.

### Osteophyte GUIs

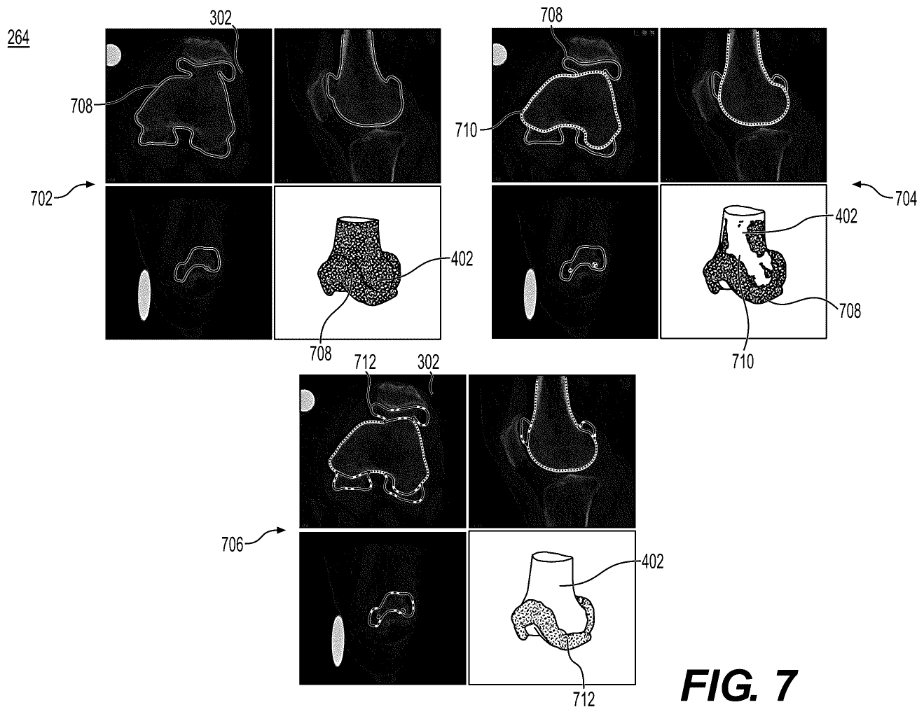

Referring to FIGS. 2 and 7, an osteophyte may be a bone spur that develops on a bone. Osteophyte volume may refer to a total volume of osteophytes on a bone or a specific portion of a bone. The osteophyte detection algorithm 60 may be configured to detect or recognize one or more osteophytes at a target bone, joint, or portion of a bone in acquired images of the imaging data 1010, and determine or calculate one or more osteophyte parameters from the preoperative data 1000 (including the acquired images in the imaging data 1010). The osteophyte parameters may relate to osteophyte detection or osteophyte dimensions or geometry (e.g., position, volume, thickness, area or compartment occupied) of one or more detected osteophytes in one or more target joints. For example, osteophyte thickness may be cross-sectional distance orthogonal to bone surface.

The one or more osteophyte parameters may include an osteophyte location, an osteophyte number, osteophyte volumes at predetermined locations, osteophyte areas across different directions (e.g., medial or lateral), an average or mean osteophyte volume, changing or progressing osteophyte volume, impingement data, impingement angles, impingement data based on a predicted or determined implant, etc. For example, the osteophyte detection algorithm 60 may determine one osteophyte volume, value or parameter per relevant bone (e.g., three in a knee joint). The osteophyte detection algorithm 60 may assess one or more of these osteophyte parameters at one or more bones (e.g., femur or tibia) and/or various anatomical compartments (e.g., anterior lateral, anterior medial, central lateral, central medial, posterior lateral, posterior medial, medial tibiofemoral (MT), lateral tibiofemoral (LT), medial patellofemoral (MP), and/or a lateral patellofemoral (LP)) of one or more bones (e.g., tibia and femur). The osteophyte detection algorithm 60 may also be configured to predict osteophyte volume or progression based on other preoperative data 1000, such as kinematics data or activity level data.

The osteophyte detection algorithm 60 may, based on supplemental patient data 1030, determine whether osteophyte volume (e.g., total osteophyte volume or an osteophyte volume of a specific region or osteophyte) is increasing or decreasing based on a comparison of previously measured osteophyte volumes and/or based on a comparison of imaging data from previous image acquisitions. The osteophyte detection algorithm 60 may further determine, predict, or diagnose a disease state or a disease progression (e.g., osteoarthritis or OA) based on the determined osteophyte parameters.

Based on the determined osteophyte parameters, the osteophyte detection algorithm 60 and/or the one or more algorithms 90 collectively may be used to determine one or more of the outputs 2000. For example, the osteophyte detection algorithm 60 may determine that an osteophyte volume of a patient is progressing over time and/or is larger than a predetermined threshold, and predict a certain (e.g., longer) procedure time 2010 accordingly, certain steps in the procedure plan 2020, etc. In addition, the osteophyte detection algorithm 60 may determine predicted outcomes 2080 (e.g., cartilage loss) and patient anatomy representations 2090 that include the detected osteophytes or that otherwise indicate osteophyte parameters.

The GUIs 250 may include a ninth GUI 264 including a plurality of screens 702, 704, 706 illustrating an osteophyte detection process by the osteophyte detection algorithm 60 so that a user (e.g., practitioner) may supervise detection.

The plurality of screens 702, 704, and 706 may include one or more first screens 702, which may display an acquired image 302 and/or an artificial model 402 of at least a target bone, along with an outer boundary of the target bone. The one or more first screens 702 may include, for example four screens displayed simultaneously or on different screens.

As exemplified in FIG. 7, the first screen 702 may display a knee joint (e.g., including a tibia, femur, and/or patella) shown in a plurality of acquired images 302 showing the knee joint at various views (top, side, perspective, enlarged/magnified, etc.). The osteophyte detection algorithm 60 may determine an outer boundary 708 of a target bone, such as an outer cortical bone of a target bone (e.g., femur). The first screen 702 may depict the determined outer boundary, which may be in a bright color (e.g., green) for visibility on the acquired images 302, which may appear in black and white or grayscale. The first screen 702 may also depict an artificial model 402 of the bone visually indicating the outer boundary 708, which may also appear in the same color as in the acquired image 302.

The second screen 704 may display the same acquired images 302 and artificial model 402 as the first screen 702, except may further display an osteophyte-free boundary or surface 710 determined by the osteophyte detection algorithm 60, in addition to continuing to display the outer boundary 708. The osteophyte-free boundary 710 may be displayed in a color that is different from the color of the outer boundary 708 (e.g., yellow).

The third screen 706 may display detected osteophytes 712, which may be determined as a function of (e.g., by subtracting or determining a difference between) the osteophyte-free boundary 710 and the outer boundary 708. The osteophytes 712 may be displayed on the same acquired images 302 and artificial model 402 as the first screen 702 and the second screen 704, but may not necessarily display the outer boundary 708 and the osteophyte-free boundary 710. The osteophytes 712 may appear in a color that is different from the colors of the outer boundary 708 and the osteophyte-free boundary 710 (e.g., red).

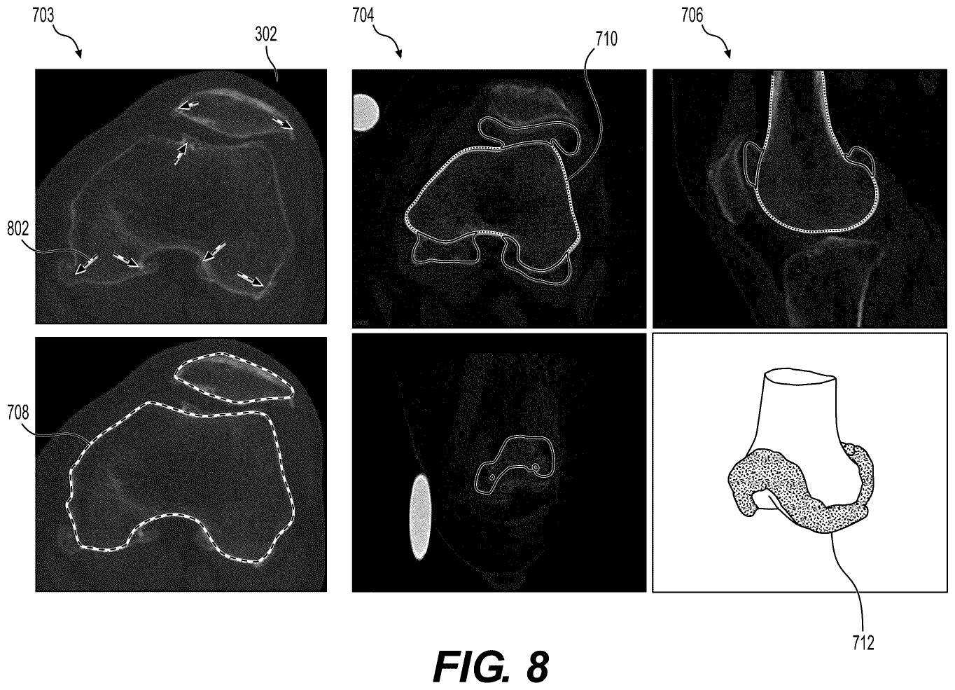

FIG. 8 illustrates a segmentation process that the osteophyte detection algorithm 60 may use and/or perform. As an alternative, some of the segmenting steps may be manually performed by a practitioner (e.g., by interacting with the ninth GUI 264).

Referring to FIGS. 2 and 8, the ninth GUI 264 may include a fourth screen 703 showing recognized or detected features (e.g., bone landmarks) by the osteophyte detection algorithm 60 to prepare for segmentation. The fourth screen 703 may include arrows or other indicators 802 that overlay the acquired image 302 and indicate bone landmarks and/or other locations that may flag boundary locations. The image analysis system 10 may determine, via osteophyte detection algorithm 60, positions of these indicators 802, and/or a practitioner may interact and/or engage with the fourth screen 703 and/or the ninth GUI 264 to manually input (e.g., via touchscreen and stylus, keyboard and mouse, or other input devices) the indicators 802.

Similar to the first screen 702, the osteophyte detection algorithm 60 may determine one or more outer boundaries 708 of one or more target bones based on the indicators 802. The osteophyte detection algorithm 60 may, for example, use statistical modelling, machine learning, autosegmentation technology, etc. The osteophyte detection algorithm 60 may be a machine learning or artificial intelligence model trained on manually segmented images that exclude osteophytes. The osteophyte detection algorithm 60 may therefore be referred to as an osteophyte-free model. The osteophyte detection algorithm 60 may have learned image features that characterize osteophytes to differentiate between osteophytic and non-osteophytic bone to identify one or more osteophyte-free bone surfaces in an image (e.g., CT image). The osteophyte detection algorithm 60 may be configured to autosegment an osteophytic bone surface (such as a cortical bone including any osteophytes), and autosegment an osteophyte-free bone surface (which may include a same cortical bone but excludes osteophytes). When comparing these two autosegmented surfaces, the osteophytic bone surface may be coincident or larger than the osteophyte-free bone surface.

The fourth screen 703 may depict the determined outer boundary 708, which may be in a bright color (e.g., yellow) for visibility on the acquired images 302, which may appear in black and white or grayscale. The fourth screen 703 may be in addition to or an alternative to the first screen 702. The second screen 704 and third screen 706 may follow as the osteophyte detection algorithm 60 progresses through a segmentation method to display osteophytes 712 (e.g., by subtracting an osteophyte-free boundary 710 from outer boundary 708).

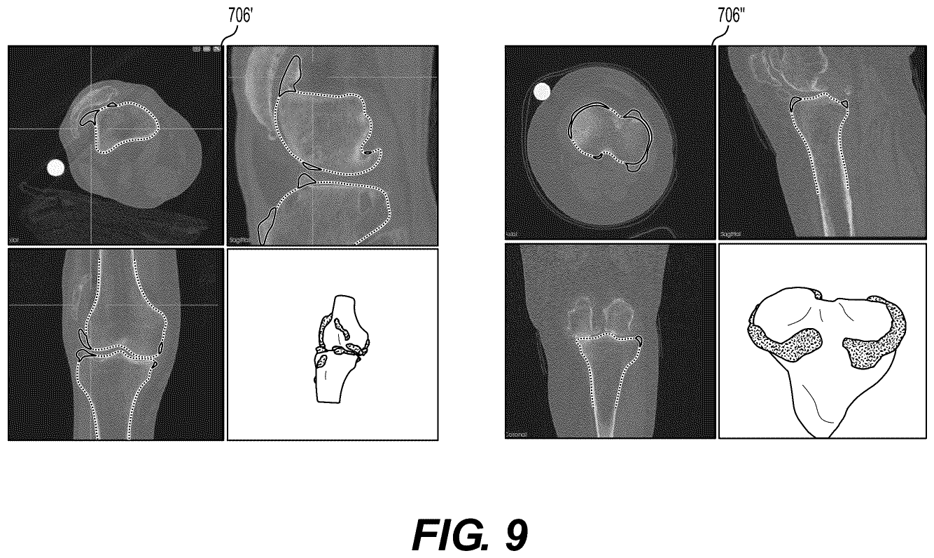

The first screen 702 and/or fourth screen 703, second screen 704, and third screen 706 may be repeated for various views of a target bone and/or various legs. For example, as shown in FIG. 9, a third screen 706′ may be displayed for a right knee joint, and a third screen 706″ may be displayed for a left knee joint.

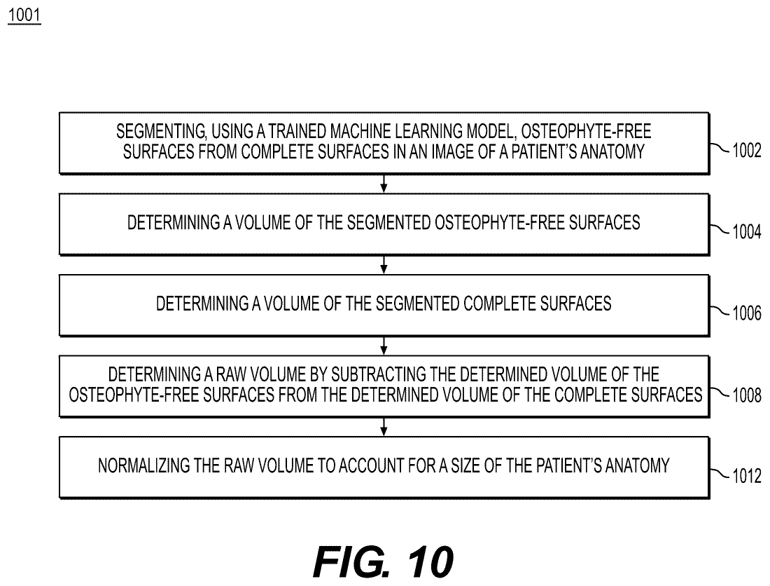

FIG. 10 shows an exemplary method or algorithm 1001 that the image analysis system 10 (e.g., via osteophyte detection algorithm 60) may execute to determine an osteophyte volume for a target bone. Referring to FIGS. 7 and 10, the method 1001 may include a step 1002 of segmenting, using a trained machine learning model, osteophyte-free surfaces or boundaries from complete surfaces (or outer boundaries) of a received or acquired image. This step 1002 of segmenting may be visualized in the second screen 704, which may show the outer boundary 708 (or complete surface) in a different color than the osteophyte-free boundary 710. The segmenting step 1002 may be performed by comparing the acquired image to a plurality of previously acquired images of previous patients and/or to previous models generated for the previous patients. The previous patients may exemplify healthy conditions or patients. The segmenting step 1002 may be performed using active appearance models (AAMs) and/or other image processing techniques configured for accurate cartilage and bone segmentations using large datasets. The segmenting step 1002 may include using a second-stage refinement using convolutional neural network machine-learning. The segmenting step 1002 may be performed using a machine learning model which was trained on manual segmentations of a separate set of many (e.g., over 1,000) preoperative acquired images (e.g., CT images) for the target bone or joint (e.g., knee joint). AAMs may intrinsically contain a dense set of anatomically corresponded landmarks, which may be used to consistently align surfaces in 3D, correcting for pose (translation and rotation), size, and shape to create spatially consistent osteophyte regions.

In step 1002, in the context of a knee joint, the femoral and tibial osteophyte-free surfaces may be segmented using an independent osteophyte-free AAM. In CT images, an original “pre-morbid” surface may be viewed (e.g., as in fourth screen 703 in FIG. 8) and the osteophyte-free surface may be manually segmented with visual interpolation of the osteophyte-free bone surface at some points (e.g., as shown by indicators 802 on fourth screen 703). The osteophyte-free models used may have been trained on many knees (e.g., over 100 or between 100 and 150) selected to include a wide variety of knee OA pathology, with manual segmentation supervised by a practitioner with years (e.g., 10-15 years) of relevant segmentation experience.

The method 1001 may include a step 1004 of determining or calculating a volume of the segmented osteophyte-free surfaces and a step 1006 of determining a volume of the segmented complete surfaces. Steps 1004 and 1006 may, for example, be determined or approximated based on calculated areas within the outer boundary 708 (for step 1006) and the osteophyte-free boundary 710 (for step 1004) for a plurality of different acquired images of different views of the target bone, but aspects disclosed herein are not limited. Steps 1004 and 1006 may be based on prior volume determinations for prior acquired images, and the osteophyte detection algorithm 60 may refine its determinations in steps 1004 and 1006 for improved accuracy. The method 1001 may include a step 1008 of determining a raw volume by subtracting the determined volume of the osteophyte-free surfaces from the determined volume of the complete surfaces. The method 1001 may include a step 1012 of normalizing the raw volume to account for a size of the patient's anatomy. A size of the patient's anatomy may be a separate input 1000 (e.g., patient data 1020) and/or inferred from other acquired images or models.

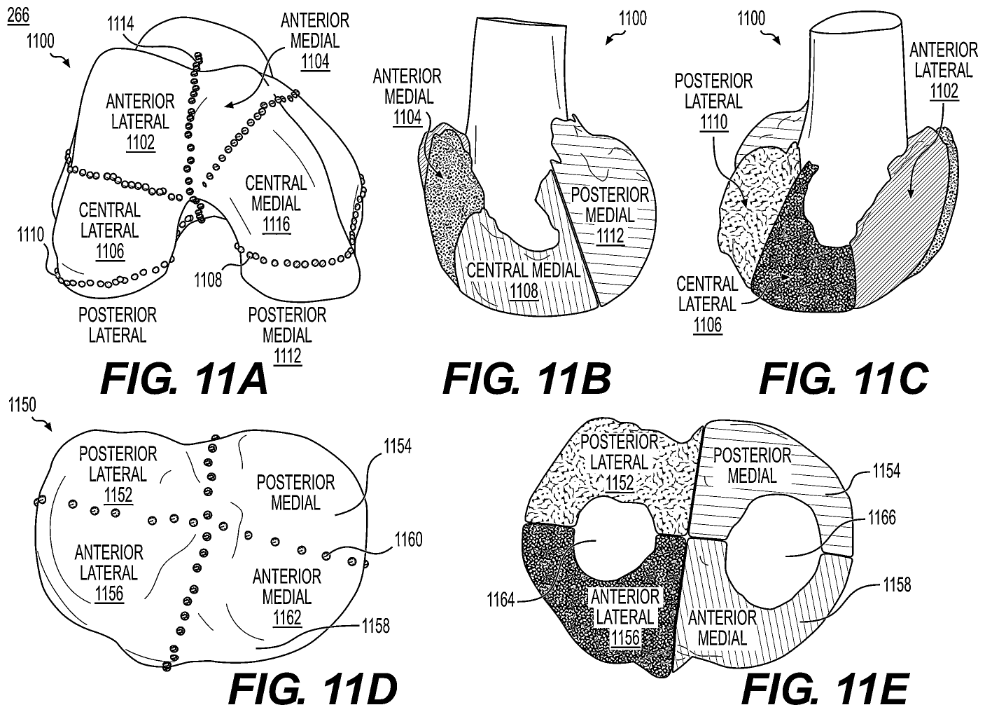

Referring to FIGS. 11A through 11E, the one or more GUIs 250 may include a tenth GUI 266 showing a three dimensional model of a target bone or joint, including osteophytes, divided into different anatomical compartments. The osteophyte detection algorithm 60 may detect osteophytes relative to these different anatomical compartments. FIGS. 11A through 11C show various views of a representative model of a femur 1100 and FIGS. 11D and 11E show various views of a representative model of a tibia 1150. Although all views 11A through 11E appear on a same screen, each may be enlarged and/or occupy a different screen.

FIG. 11A shows a bottom view of the representative model of the femur 1100, or a “femur model.” The femur model 1100 may be divided into an anterior lateral compartment 1102, an anterior medial compartment 1104, a central lateral compartment 1106, a central medial compartment 1108, a posterior lateral compartment 1110, and a posterior medial compartment 1112. In FIG. 11A, the anterior lateral compartment 1102, anterior medial compartment 1104, central lateral compartment 1106, central medial compartment 1108, posterior lateral compartment 1110, and posterior medial compartment 1112 may be divided by color coded lines 1114, which may be dashed or dotted. In some examples, labels or text 1116 may appear identifying the compartments. In some examples, color coded lines 1114 may visually indicate (e.g., via color or density) a determined joint-space width (e.g., by joint-space width algorithm 50) in each compartment.

FIG. 11B shows a medial side of the femur model 1100. In FIG. 11B, the anterior medial compartment 1104, central medial compartment 1108, and the posterior medial compartment 1112 may be color coded in different colors for visibility. FIG. 11C shows a lateral side of the femur model 1100. In FIG. 11C, the anterior lateral compartment 1102, the central lateral compartment 1106, and the posterior lateral compartment 1110 may be color coded in different colors for visibility.

FIG. 11D shows a top view of the representative model of the tibia 1150, or a “tibia model.” The tibia model 1150 may be divided into a posterior lateral compartment 1152, a posterior medial compartment 1154, an anterior medial compartment 1156, and an anterior medial compartment 1158. In FIG. 11D, the posterior lateral compartment 1152, posterior medial compartment 1154, anterior medial compartment 1156, and anterior medial compartment 1158 may be divided by color coded lines 1160, which may be dashed or dotted. In some examples, labels or text 1162 may appear identifying the compartments. FIG. 11E shows a top view of the tibia model 1150 where, instead of color coded lines 1160, the posterior lateral compartment 1152, posterior medial compartment 1154, anterior medial compartment 1156, and anterior medial compartment 1158 are color-coded. In FIG. 11E, the medial and lateral meniscuses may be colored, while a central lateral surface or recess 1164 and a central medial surface or recess 1164 configured to support articular cartilage may remain a neutral color or bone colored.

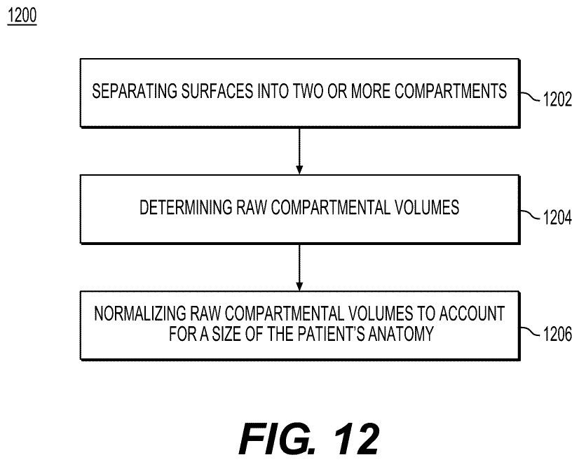

Referring to FIGS. 2 and 12, the osteophyte detection algorithm 60 and/or image analysis system 10 may execute a method or algorithm 1200 to determine osteophyte location and/or volume in different anatomical compartments, such as the anatomical compartments illustrated in FIGS. 11A-11E for a knee joint. The method 1200 may include a step 1202 of separating recognized surfaces in an acquired image of a target bone or joint into two or more compartments, such as medial and lateral compartments, or such as anterior or posterior compartments. Step 1202 may include comparing the acquired image to a plurality of images or models of the target bone or joint in previous or healthy patients. Step 1202 may also be performed using a semi-quantitative MRI Osteoarthritis Knee Score (MOAKS) scoring system. In the context of a knee joint, in step 1202, a femur may be divided into three compartments—anterior, central, and posterior—by referencing anterior and posterior meniscal edges, using a bone and meniscal model of healthy individuals. The tibia may be separated into anterior and posterior regions of equal size. The divisions may be displayed (e.g., on tenth GUI 266 as shown in FIG. 11). In segmenting the compartments, a partition or division line between compartments may be aligned using anatomically corresponded landmarks from AAM segmentation (e.g., from the AAM segmentation performed in method 1001 of FIG. 10).

The method 1200 may include a step 1204 of determining raw compartmental volumes of each anatomical compartment. The raw compartmental volume may be based on a previously determined raw volume from method 1001 and/or calculated using a segmentation process. The raw compartment volume for a compartment may be a volume of all osteophytes in that compartment.

The method 1200 may include a step 1206 of normalizing each raw compartmental volume to account for a size of the patient's anatomy (e.g., bone size). A size of the patient's anatomy may be a separate input 1000 (e.g., patient data 1020) and/or inferred from other acquired images or models. For example, in the context of a knee joint, compartmental volumes may be normalized for bone size, multiplying raw values by the ratio Rb=volume of test bone/mean volume of all bones, using a distal femoral volume and a proximal tibial volume of osteophyte-free surfaces.

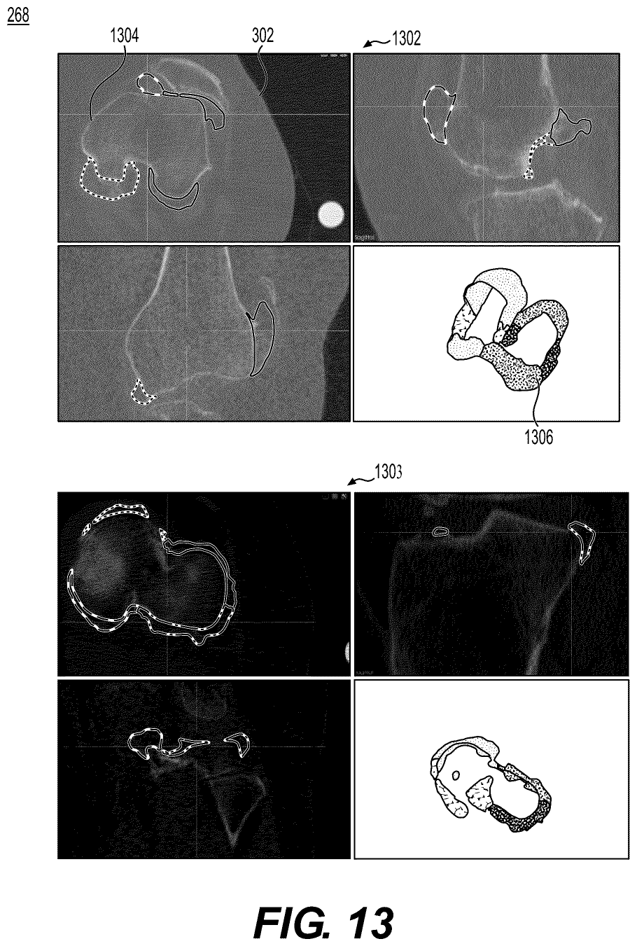

Referring to FIG. 13, the one or more GUIs 250 may include an eleventh GUI 268 configured to display osteophyte models color-coded based on compartments (e.g., medial and lateral). For example, the eleventh GUI 268 may include a first screen 1302 for a first target bone (e.g., femur) of a joint and a second screen 1303 for a second target bone (e.g., tibia) of the joint. Each of the first screen 1302 and the second screen 1303 may include one or more acquired images 302 of one or more views of the first target bone or the second target bone, respectively, that display one or more boundary lines 1304. The boundary lines 1304 may be determined using a segmentation process and represent an outer boundary and osteophyte-free boundaries. The boundary lines 1304 may be color-coded depending on an anatomical compartment (e.g., medial or lateral, or anterior or posterior). Each of the first screen 1302 and the second screen 1303 may include a representative model 1306 of the osteophytes, separate from an osteophyte-free surface or base bone, that is color coded according to the anatomical compartments (e.g., medial or lateral, or anterior or posterior).

The osteophyte detection algorithm 60 may determine features within a bone, and determine, based on the detected features within the bone, osteophytes and other related parameters outside the bone. For example, the osteophyte detection algorithm 60 may detect areas of radiolucency on the raw images and/or in the imaging data 1010, and determine osteophytes outside the bone based on the detected areas.

### B-Score GUIs

Referring to FIG. 14, B-score may be a type of score or scoring system based on and/or quantifying a shape of a femur or knee joint. B-score may be a holistic, average, or overall score indicating an overall assessment of the femur and/or the knee, but knees having different specific complications or deformities may result in similar B-scores. The B-score may be based on how the shape of the femur compares to knee shapes of those with OA and knee shapes of those who do not have OA, and may be determined using, for example, statistical shape modelling (SSM) or other processes. B-score may be a continuous, quantitative variable, which may be used to quantify overall amount of OA damage in the knee, and to measure progression in longitudinal studies. In other examples, such as with procedures relating to any anatomical joint, a similar measurement and scoring system which is based on quantifying a shape and/or other measurements one or more bones of a joint, similar to the B-score. Some procedures that may utilize a similar system, for example display an associate B-score or other metric to quantify osteophyte volume, ligament laxity, range or motion, joint health, or other parameters described herein, are hip procedures, ankle procedures, spine procedures, shoulder procedures, elbow procedures, hand procedures, and the like. Any of the measuring and scoring systems described in the present application may be used in other surgical procedures, such as other orthopedic surgeries or any other surgical procedure, in addition to knee procedures.

As OA progresses, each bone may exhibit a characteristic shape change, involving osteophyte growth around cartilage plates, and a spreading and flattening of a subchondral bone. A femur shape change may increase regardless of an anatomical compartment affected, and may be more sensitive to change than the tibia and patella. The B-score may represent a distance along the “OA” shape change in the femur bone. B-score may correlate to total osteophyte volume.

In some examples, a B-score may be recorded as a z-score, similar to a T-score in osteoporosis, which may represent units of standard deviation (SD) of a healthy population, with 0 defined as ae mean of a healthy population. Values of −2 to +2 may represent a healthy population, whereas values above +2 may fall beyond the healthy population.

The B-score algorithm 70 may be configured to determine a B-score from the acquired images 302. The B-score may be based in part on, or correlate to, OA progression, where a B-score of 0 may correlate to and/or indicate a mean femur shape of those who do not have OA. Further details of how B-score is calculated may be found in “Machine-learning, MRI bone shape and important clinical outcomes in osteoarthritis: data from the Osteoarthritis Initiative” by Michael A. Bowes, Katherine Kacena, Oras A. Alabas, Alan D. Brett, Bright Dube, Neil Bodick, Philip G Conaghan published Nov. 13, 2020, and in “3D Bone Shape from CT-Scans Provides an Objective Measure of Osteoarthritis Severity: data from the IMI-APPROACH study” by James M. Burlison, Michael A. Bowes, Philip G. Conaghan, and Alan D. Brett published December 2024, both of which are incorporated by reference herein in their entirety. Aspects disclosed herein are not limited to such a B-score, however. For example, the B-Score algorithm 70 may additionally and/or alternatively calculate other scores or quantifications of other bone shapes based on how they compare to bone shapes of those having a particular disease.

The B-score algorithm 70 may be configured to detect or recognize one or more target bones or joint (e.g., femur), detect or recognize a shape of the target bone or joint, and/or determine or calculate one or more shape score parameters from the preoperative data 1000 (e.g., imaging data 1010) relating to the shape of the target bone and/or how that shape compares with prior patients having a particular disease. For ease of description, an example where the B-score algorithm 70 calculates one or more B-score parameters in connection to a knee and/or femur will be described. The one or more B-score parameters may include B-scores at different times or in different images, an average or mean B-score, and/or a changing or progressing B-score. The B-score algorithm 70 may also be configured to predict a future B-score or B-score progression based on other preoperative data 1000, such as kinematics data or activity level data.

The B-score algorithm 70 may, based on supplemental patient data 1030, determine whether a B-score for a particular femur (e.g., left femur) or both femurs is increasing or decreasing based on a comparison of previously measured B-scores and/or based on a comparison of imaging data from previous image acquisitions. The B-score algorithm 70 may further determine, predict, or diagnose a disease state or a disease progression (e.g., osteoarthritis or OA) based on the determined B-score and/or B-score progression.

As shown in FIG. 14, the one or more GUIs 250 may include a twelfth GUI 270. The twelfth GUI 270 may include a plurality of screens or frames showing OA progression for a patient. Each screen may include a representative model 1402 of a patient's femur determined by the B-score algorithm 70 and/or the image analysis system 10 and text 1404 or another indicator indicating the B-score for that model. The plurality of screens or frames may be implemented as a video progression so that the twelfth GUI 270 shows a progression of OA in video format. Some of the later frames may show a predicted progression if, for example, the patient does not undergo treatment or, alternatively, a predicted improvement if the patient undergoes treatment (e.g., procedure plan 2020).

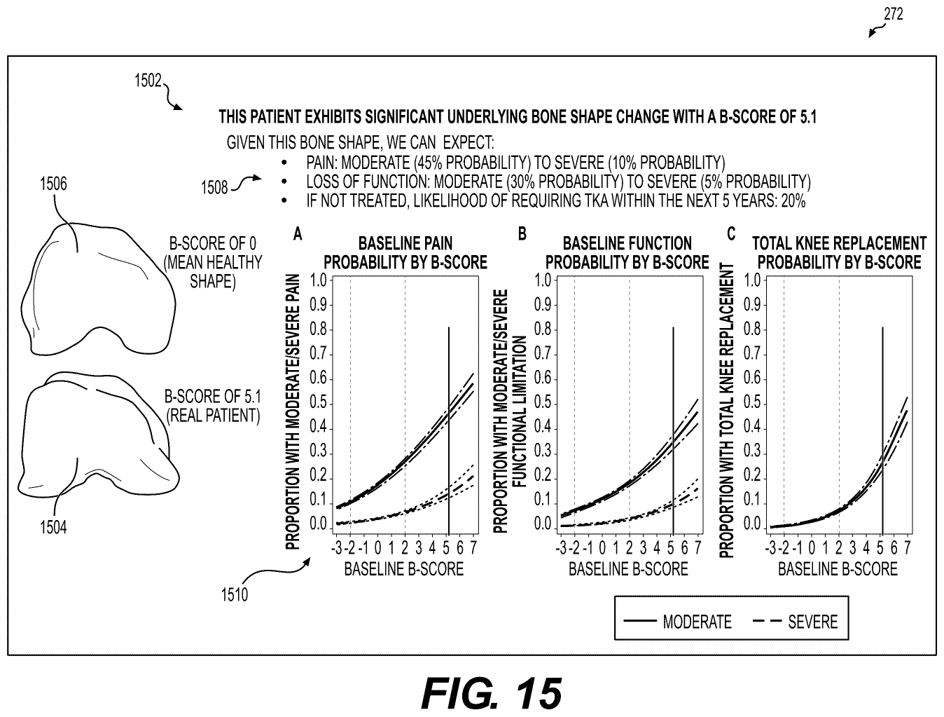

Referring to FIG. 15, based on the determined B-score, the B-score algorithm 70 and/or the one or more algorithms 90 collectively may be used to determine one or more of the outputs 2000, such as predicted outcomes 2080, which may be displayed on a thirteenth GUI 272. For example, the B-score algorithm 70 may calculate a patient's current B-score, and display the current B-score on the thirteenth GUI 272 as text or another indicator 1502. The B-score algorithm 70 and/or the image analysis system may generate a representative model 1504 of the patient's femur using the determined B-score and/or other parameters or input (e.g., acquired image 302 and/or stored models), and may also generate a comparative representative model 1506 displaying a healthy femur having a B-score of 0 and/or displaying a prediction of how the patient's femur would look with a B-score of 0.

The B-score algorithm 70 may determine predicted outcomes 2080 such as a predicted perceived pain level and/or predictions as to the likelihood of severe or moderate pain if the patient continues without treatment (or, alternatively, predictions for improvement based on the prediction plan 2020), predicted function loss and/or predictions as to the likelihood of severe or moderate function loss if the patient continues without treatment (or, alternatively, predictions for improvement based on the prediction plan 2020), and a prediction and/or likelihood that a total joint replacement surgery and/or a total arthroplasty (e.g., total knee arthroplasty or TKA) will be required in a predetermined future time (e.g., within the next 5 years). Such predicted outcomes 2080 may be described and/or explained in a text section 1508 of the thirteenth GUI 272. The predicted outcomes 2080 may also be illustrated in one or more charts or graphs 1510. For example, when the predicted outcomes 2080 are expressed in terms of a likelihood percentage (e.g., a likelihood of severe pain or moderate pain), these predictions may be graphed as a function of B-score.

Referring to FIG. 16A, the one or more GUIs 250 may include a fourteenth GUI 274 configured to display predicted outcomes 2080. The fourteenth GUI 274 may include a scale 1602 or gradient bar having an indicator 1604 to indicate a B-score calculated by the B-score algorithm 70. The scale 1602 may be similar to scale 608 (FIG. 6A) in using colors and/or shading corresponding to a severity of OA progression or a health level associated with the B-score. The fourteenth GUI 274 may also include a B-score evolution video 1606 showing an actual and/or predicted OA progression or femur shape for a patient, similar to the twelfth GUI 270 (FIG. 14). The B-score evolution video 1606 may include a representative model 1608 of a patient's femur determined by the B-score algorithm 70 and a user input 1610 (e.g., play, pause, fast forward, rewind, zoom, volume, and/or timeline buttons) to control a progression of the B-score evolution video 1606.

The fourteenth GUI 274 may also include predicted outcomes 2080 such as a predicted perceived pain level and/or predictions as to the likelihood of severe or moderate pain if the patient continues without treatment (or, alternatively, predictions for improvement based on the prediction plan 2020), predicted function loss and/or predictions as to the likelihood of severe or moderate function loss if the patient continues without treatment (or, alternatively, predictions for improvement based on the prediction plan 2020), and a prediction and/or likelihood that a total joint replacement surgery and/or a total arthroplasty (e.g., total knee arthroplasty or TKA) will be required in a predetermined future time (e.g., within the next 5 years). For example, the fourteenth GUI 274 may display a probability of perceived pain 1612 (such as using the visual analog scale out of 10 or other value or score system). The probability of perceived pain 1612 may be expressed as a percentage, such as a probability of moderate pain 1614 and a probability of severe pain 1616. The B-score algorithm 70 may calculate a probability of moderate pain 1614 based on a predicted perceived pain of greater than a first predetermined pain score (e.g., a VAS score of 4). The B-score algorithm 70 may calculate a probability of severe pain 1616 based on a predicted perceived pain of greater than a second predetermined pain score (e.g., a VAS score of 8). The probability of moderate pain 1614 may be displayed in and/or bordered by a first color associated with moderate pain (e.g., yellow), and the probability of severe pain 1616 may be displayed in and/or bordered by a second color associated with severe pain (e.g., red).

The fourteenth GUI 274 may display a probability of a loss of function 1618 (such as using the Knee Injury or Osteoarthritis Outcome Score or KOOS, or the Western Ontario and McMaster Universities Osteoarthritis Index's or WOMAC scale out of 64). The probability of loss of function 1618 may be expressed as a percentage, such as a probability of moderate loss of function 1620 and a probability of severe loss of function 1622. The B-score algorithm 70 may calculate a probability of moderate loss of function 1620 based on a predicted perceived loss of function of greater than a first predetermined loss of function score (e.g., a WOMAC score of 20). The B-score algorithm 70 may calculate a probability of severe loss of function 1622 based on a predicted perceived pain of greater than a second predetermined loss of function score (e.g., a WOMAC score of 8). The probability of moderate loss of function 1618 may be displayed in and/or bordered by a first color associated with moderate loss of function (e.g., yellow), and the probability of severe loss function 1622 may be displayed in and/or bordered by a second color associated with severe loss function (e.g., red).

The B-score algorithm 70 may determine and/or predict (or be used to determine and/or predict) other outcomes 2000 such as the procedure time 2010 to execute the procedure plan 2020. The B-score algorithm 70 may use both a determined B-score and other patient data 1020, and may determine different relationships based on different characteristics of a patient in the patient data 1020. For example, patients belonging to the U.S. population that have a higher B-score may be associated with longer procedure times 2010, while patients belonging to EU populations that have a higher B-score may be associated with shorter procedure times 2010. Thus, the B-score algorithm 70 and/or image analysis system 10 may determine a longer procedure time 2010 based on a higher B-score and a patient nationality of U.S. and a shorter procedure time 2010 based on a higher B-score and a patient nationality of an EU country. Other factors (e.g., from patient data 1020) may change certain relationships such that the image analysis system 10 and/or the B-score algorithm 70 may determine certain relationships between higher or lower B-scores combined with certain patient data 1020.

Referring to FIG. 16B, the fourteenth GUI 274 may be implemented as one of the cards or widgets 409 displayed on third GUI 255. For example, the plurality of widgets 409 may include the B-score card 414 described with reference to FIG. 4. A user may click the B-score card 414 to display the fourteenth GUI 274. FIG. 16B shows an example where the fourteenth GUI 274 may appear as a popup or as a separate frame of the third GUI 255, but aspects disclosed herein are not limited. For example, clicking the B-score card 414 may bring up the fourteenth GUI 274 as a full screen GUI, as shown in FIG. 16A, and/or show a magnified or enlarged view of B-score card 414 on third GUI 255 before it is clicked. Alternatively or in addition thereto, clicking the B-score card 414 may bring up an abbreviated version of fourteenth GUI 274 that displays some, but perhaps not all, features of fourteenth GUI 274. For example, the B-score card 414, when clicked, may display gradient bar or scale 1602, a determined B-score (e.g., 5.1), and representations of bones (e.g., femurs) at various B-scores for comparison, such as below the determined B-score (e.g., 0 and 3) and above the determined B-score (e.g., 7).

Referring to FIG. 16C, the fourteenth GUI 274 may, upon clicking, rotate or flip to display a “back” of the B-score card 414, which may display textual information, videos, etc. or other additional information or analysis (e.g., determinations by the one or more algorithms 90) regarding B-score or bone shape. For example, a back of the B-score card 414 may display B-score video 1606 and/or an analysis of a change in B-score. The B-score card 414 may provide an assessment of a change in shape based on a 3D model (e.g., artificial model 402), such as that the 3D femur bone has a consistent shape change in knee osteoarthritis, and that a shape has been recorded as a B-score. The B-score card 414 may describe patient-specific variations in shape and/or curvature of the bone which may not be reflected by the overall B-score.

Although FIGS. 15 and 16A through 16C exemplify predictions related to pain and loss of function, the displayed predicted outcomes 2080 are not limited to pain and loss of function. For example, image analysis system 10 may predict a stress level, anxiety level, and/or mental health status of the patient, a recovery time, a risk of complications during the procedure (e.g., breathing difficulties and/or blood flow or heart rate complications), a risk of infection, a likelihood of revision surgery, and/or an increased difficulty rating based on a comparison of the determined joint space width by the joint space width algorithm 50 with a planned implant size in the determined procedure plan 2020, based on a narrower (or wider) joint space width determined by the joint space width algorithm 50, based on joint space narrowing over time determined by the joint space width algorithm 50, based on an osteophyte volume or osteophyte number determined by the osteophyte detection algorithm 60, based on a progressing osteophyte volume determined by the osteophyte detection algorithm 60, based on a B-score or progressing B-score (or alternatively, a B-score outside of a predetermined range) determined by the B-score algorithm 70, based on a severe deformity detected by the alignment/deformity algorithm 80, based on an OA progression determined using the one or more algorithms 90, based on impingement data calculated using parameters determined from the joint space width algorithm 50, the osteophyte detection algorithm 60, and/or the alignment/deformity algorithm 80, a larger bone-to-tissue ratio, PPT, and/or PTT, etc. With respect to cartilage loss, the image analysis system 10 may determine a Z score or other statistical measure to determine a risk of cartilage loss. The determined predicted cartilage loss may be based on the joint space width. These predicted outcomes 2080 may be displayed on any of the GUIs 250, such as twelfth GUI 270, fourth GUI 256 (FIG. 4B), second and third GUIs 254 and 255 (FIGS. 4A through 4E), etc.

### Tissue-to-Bone GUIs

Referring to FIGS. 2, 3, and 17-19, the one or more algorithms 90 may be configured to detect or determine, from one or more acquired images 302, prepatellar thickness (PPT) and/or pretubercular thickness (PTT), a minimum distance from bone to skin, tissue-to-bone ratio, bone-to-tissue distances or values, and/or bone-to-tissue distances for PPT and/or PTT, bone-to-skin ratio, etc.

PPT and/or PTT may be a distance measurement between a bone and skin determined using the acquired images 302 (e.g., CT scans), and may be used as a proxy or alternative to a manually input BMI. In some examples, PPT and/or PTT at a joint (e.g., knee joint) may provide more precise information than BMI, which may be a whole-body measurement. The image analysis system 10 may determine certain tissue-to-bone parameters such as a bone-to-tissue ratio, PPT, PTT, and/or BMI and/or use some of these parameters as input (e.g., as patient data 1020 or from a previous output of the one or more algorithms). The image analysis system 10 may determine one or more outputs 2000 based on the determined certain tissue-to-bone parameters. For example, the one or more algorithms 90 may determine a larger procedure time 2010 based on a larger determined tissue-to-bone ratio, as practitioners may need more time to handle (e.g., cut through) a larger amount of tissue. In addition, the image analysis system 10 may determine a higher case difficulty level based on a larger bone-to-tissue ratio, PPT, and/or PTT determined by the one or more algorithms 90, as a joint (e.g., knee) may be harder to balance due to more tissue.

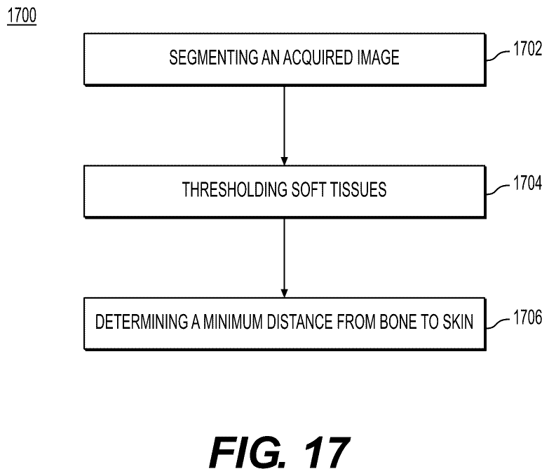

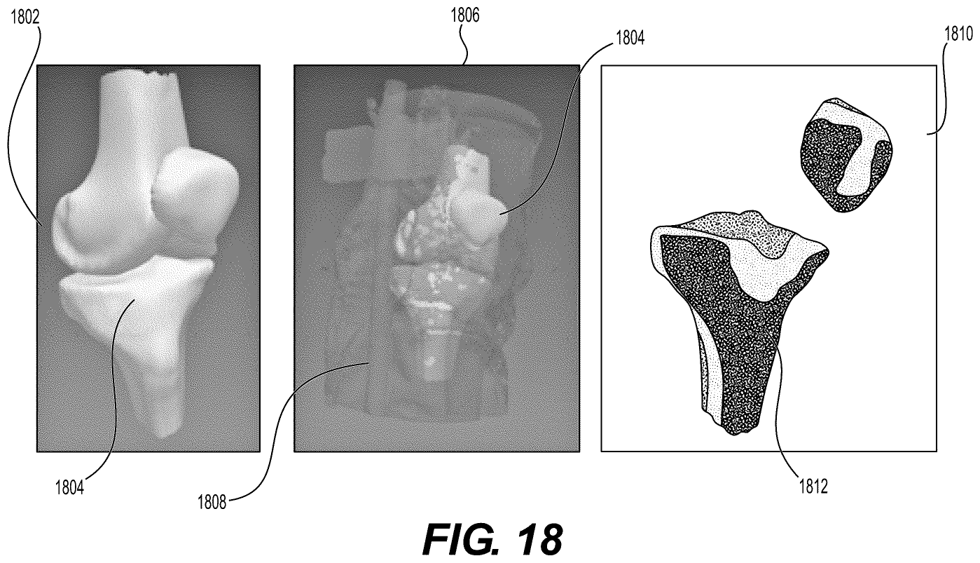

FIG. 17 describes an image processing method 1700 that the one or more algorithms 90 may use to determine tissue-to-bone parameters, and FIG. 18 shows a fifteenth GUI 276 that may display certain steps as the one or more algorithms 90 perform the image processing method 1700. The image processing method 1700 may include a step 1702 of segmenting an acquired image 302, such as a CT scan. Based on the segmenting step 1702, the fifteenth GUI 276 may display a frame 1802 showing a representative model 1804 of the patient's anatomy. The image processing method 1700 may include a step 1704 of thresholding soft tissues, which may be visualized in frame 1806 showing the representative model 1804 and a boundary 1808 encompassing the soft tissues. The image processing method 1700 may include a step 1706 of determining a minimum distance from bone to skin. For example, step 1706 may include determining the minimum distance from bone to skin at a plurality of locations on a bone, and the distances may be expressed according to a color gradient scale, which may be overlaid on the representative model 1804. Frame 1806 shows a color-coded representation 1812 of a portion of the representative model 1804 (e.g., tibia).

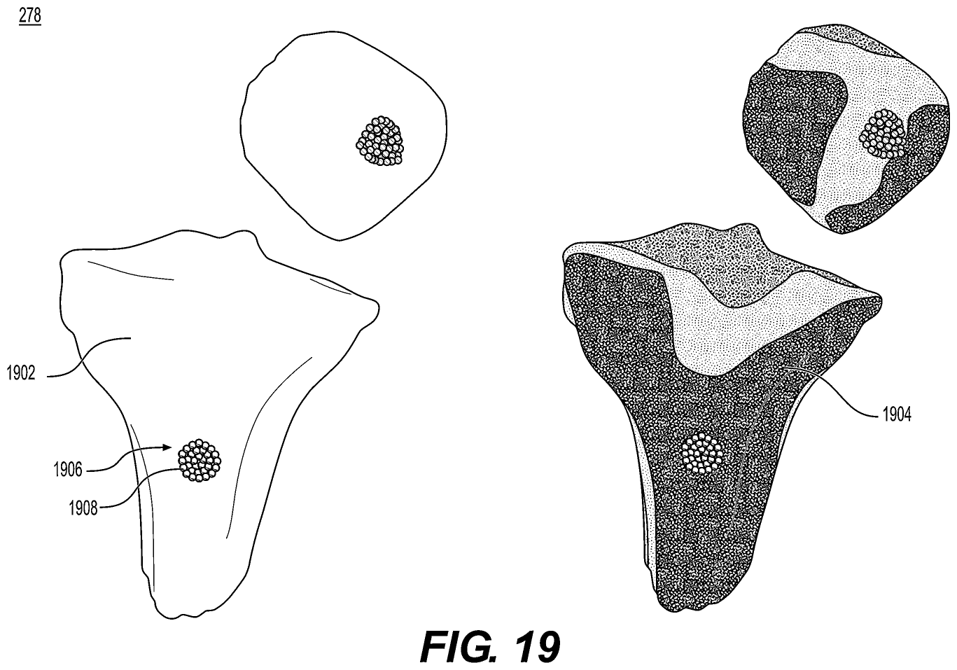

Alternatively or in addition to fifteenth GUI 276, the one or more GUIs 250 may include a sixteenth GUI 278 (FIG. 19) configured to display a representative model 1902 of at least a portion of the patient's bone adjacent to or on the same screen as a corresponding color coded representation 1904. The sixteenth GUI 278 may include one or more target or areas 1906, which may be predefined and/or recognized by the one or more algorithms 90. For example, each area 1906 may represent an area of clinical importance (e.g., generally associated with one or more particular bones, such as a knee joint, and/or for a particular patient). Each target area 1906 may include a plurality of smaller areas or points 1908. Each point 1908 may indicate a key anatomical region or point on a bone (e.g., femur, tibia and patella bones). The one or more algorithms 90 may calculate bone-to-skin distances from all of these points 1908 in these target areas 1906 to provide a bone-to-skin or soft tissue thickness value, which may be output to storage and/or displayed (e.g., on GUI 278).

### Toggling Features

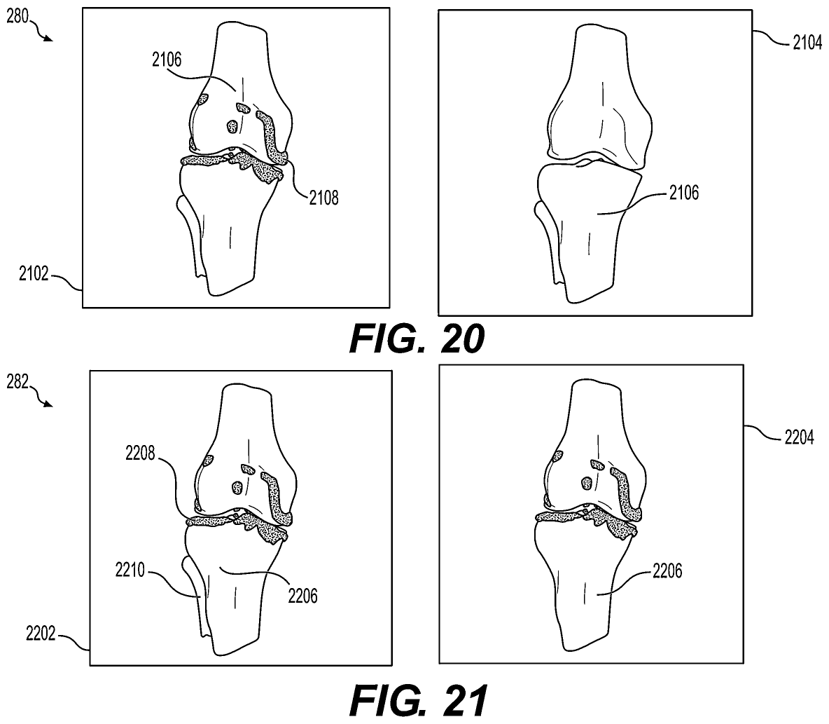

Referring to FIGS. 20-23, any of the one or more GUIs 250 may include a toggling function where certain displayed features may be selectively displayed, toggled off, or modified. Referring to FIG. 20, the one or more GUIs 250 may include a seventeenth or osteophyte toggling GUI 280 having a first screen or frame 2102 and a second screen or frame 2104. The first frame 2102 may display a representative model 2106 and one or more osteophytes 2108 determined by the image analysis system 10 using the one or more algorithms 90. A user may input a command to toggle off the osteophytes to reveal second frame 2104, which may display the representative model 2106 and may omit the one or more osteophytes 2108. The user may input the command by clicking on the osteophytes, pressing a key, touching the osteophytes or swiping on the osteophytes using a touch screen, etc. Although first and second frames 2102 and 2104 only show the representative model 2106 in seventeenth GUI 280, the toggling functionality exemplified in FIG. 20 may be applied to any GUI displaying a representative model and osteophytes, such as second and third GUIs 254 and 255 (FIGS. 4A through 4E), ninth GUI 264 (FIGS. 7-9), etc.

Referring to FIG. 21, the one or more GUIs 250 may include an eighteenth or bone portion toggling GUI 282 having a first screen or frame 2202 and a second screen or frame 2204. The first frame 2202 may display a representative model 2206 and one or more osteophytes 2208 determined by the image analysis system 10 using the one or more algorithms 90, similar to the seventeenth GUI 280. A portion 2210 of the representative model 2206 may be toggled off in the second frame 2204. As exemplified in FIG. 21, the portion 2210 of the representative model 2206 may represent a fibula. The user may input a command to toggle off the portion 2210 to reveal second frame 2204, which may display the representative model 2206 and may omit the portion 2210 (e.g., fibula), but the osteophytes 2208 may remain. Although the portion 2206 is exemplified as a fibula in FIG. 21, other portions of the bone may be toggled on and/or off, such as a patella, certain anatomical compartments, certain bone landmarks (e.g., condyle surfaces), etc. The user may input the command by clicking on the portion 2210, pressing a key, touching the osteophytes or swiping on the osteophytes using a touch screen, etc. Although first and second frames 2202 and 2204 only show the representative model 2206 in eighteenth GUI 282, the toggling functionality exemplified in FIG. 21 may be applied to any GUI displaying a representative model, such as second and third GUIs 254 and 255 (FIGS. 4A through 4E), ninth GUI 264 (FIGS. 7-9), etc.

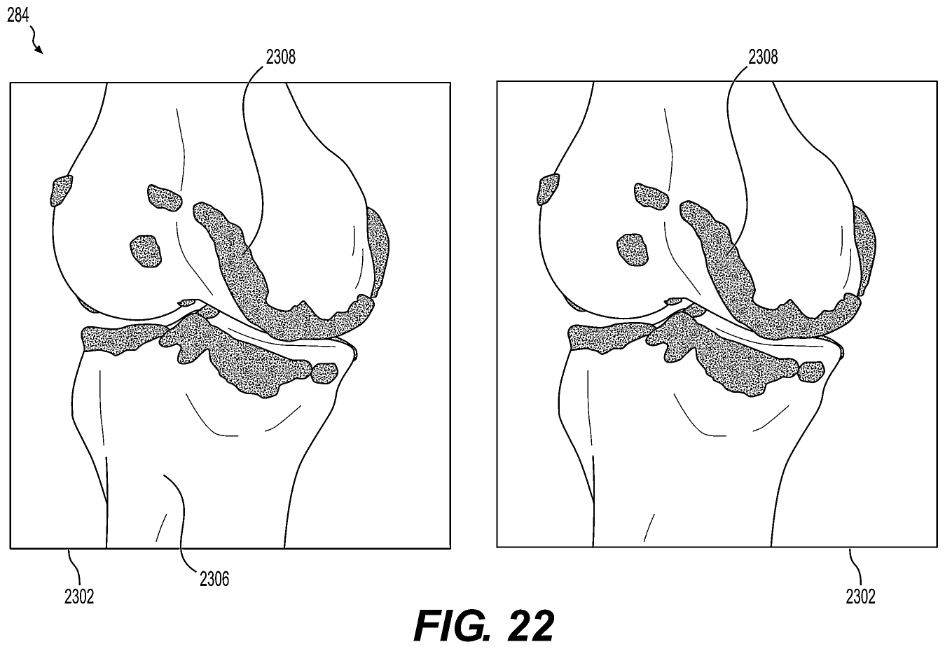

Referring to FIG. 22, the one or more GUIs 250 may include a nineteenth or opacity GUI 284 having a first screen or frame 2302 and a second screen or frame 2304. The first frame 2302 may display a representative model 2306 and one or more osteophytes 2308 determined by the image analysis system 10 using the one or more algorithms 90. The osteophytes 2308 may be displayed in a first color and/or opacity. A user may input a command to change a color and/or opacity of the osteophytes 2308 to reveal second frame 2304, which may display the representative model 2306 and the osteophytes 2308 in a selected opacity. The user may input the command by clicking on the osteophytes 2308, pressing a key, touching the osteophytes or swiping on the osteophytes using a touch screen, etc. For example, the first frame 2302 may display opaque osteophytes 2308, and the user may toggle to the second frame 2304, which may display more transparent osteophytes 2308 so that portions of the representative model 2306 under the osteophytes 2308 may be visible. Although first and second frames 2302 and 2304 only show the representative model 2306 in nineteenth GUI 284, the toggling functionality exemplified in FIG. 22 may be applied to any GUI displaying a representative model and osteophytes, such as second and third GUIs 254 and 255 (FIGS. 4A through 4E), ninth GUI 264 (FIGS. 7-9), etc.

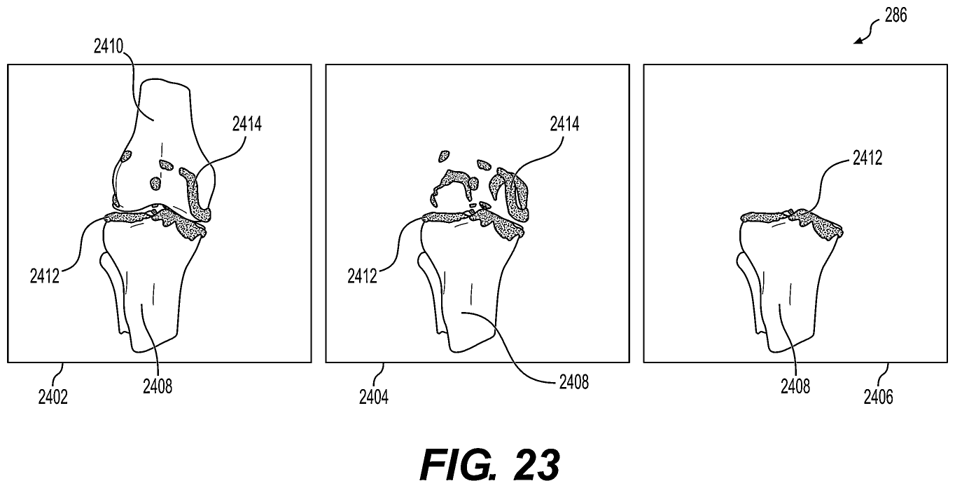

Referring to FIG. 23, the one or more GUIs 250 may include a twentieth or bone and osteophyte toggling GUI 286 having a first screen or frame 2402, a second screen or frame 2404, and a third screen or frame 2406. The first frame 2402 may display a representative model determined by the image analysis system 10 and having a first bone 2408 (e.g., tibia), a second bone 2410 (e.g., femur), one or more first osteophytes 2412 on the first bone 2408, and one or more second osteophytes 2414 on the second bone 2410. A user may input a command to toggle off any one of the first bone 2408, the second bone 2410, the one or more first osteophytes 2412, and the one or more second osteophytes 2414. For example, the second frame 2404 may toggle off the second bone 2410 such that the second frame 2404 displays the first bone 2408, the one or more first osteophytes 2412, and the one or more second osteophytes 2414, and omits the second bone 2410. Alternatively, the first bone 2408 may be toggled off. The third frame 2406 may toggle off the second osteophytes 2414 such that the third frame 2406 displays the first bone 2408 and the one or more first osteophytes 2412, and omits the second bone 2410 and the second osteophytes 2414. The user may select a feature to turn off and/or on by inputting a command, such as by clicking on the features to toggle off (e.g., first bone 2408, the second bone 2410, the one or more first osteophytes 2412, and the one or more second osteophytes 2414), pressing a key, touching the features or swiping on the features using a touch screen, etc. The toggling functionality exemplified in FIG. 23 may be applied to any GUI displaying a representative model and osteophytes, such as second and third GUIs 254 and 255 (FIGS. 4A through 4E), ninth GUI 264 (FIGS. 7-9), etc.

Any of the GUIs or functionalities described with reference to FIGS. 20-23 and the seventeenth through twentieth GUIs 280-286 may be implemented in third GUI 255 (FIG. 4B) and/or accessible via menu 418 (FIG. 4B) or other interactive features. For example, the user may click on displayed indicators 404 of osteophytes, etc. and/or bones (e.g., femur, tibia, or fibula) to toggle them on or off, and/or use menu 418 to click a switch. The menu 418 may also include a slider for opacity of the indicators 404 (e.g., osteophytes).

### Simulated Movement GUI

Referring to FIG. 24, the plurality of GUIs 250 may include a twenty-first GUI 288 to display movement of one or more bones and one or more ligaments. The twenty-first GUI 288 may allow the practitioner to assess 3D interactions where imaging modalities may produce a 2D image (e.g., X-ray) and where certain areas (e.g., a tibial plateau) may not be as visible. A procedure plan (e.g., procedure plan 2020) may call for removing chunks of bone to provide room for an implant, but may also call for leaving certain osteophytes. The twenty-first GUI 288 may allow a practitioner to assess how remaining osteophytes may cause problems with a soft tissue envelope and joint balancing (e.g., knee balancing). For example, a ligament may remain stretched if it consistently interacts with remaining osteophytes. An osteophyte near a ligament may stretch the ligament and may create gaps in some areas, such as between the ligament and bone. As another example, a close positional relationship between the ligament and remaining osteophytes may cause pain.

The twenty-first GUI 288 may display an artificial model 402 of one or more bones (e.g., knee joint) and one or more indicators 404 of a patient's osteophytes (e.g., determined by the one or more algorithms 90). The twenty-first GUI 288 may display a simulated movement of the artificial model 402, such as a simulated flexion and/or extension. The simulated movement may be determined by the one or more algorithms 90 based on prior procedure data 1050 of multiple patients and/or available simulated or statistical models. In some examples, the simulated movement may be determined by the one or more algorithms 90 using patient data 1020 (e.g., alignment data, range of motion data, etc.) and/or imaging data 1010. In some examples, the twenty-first GUI 288 may display the patient data 1020 and/or other data 1000 used to determine the simulated movement.

The twenty-first GUI 288 may also display a ligament 2502 (e.g., medial collateral ligament or MCL) on the artificial model 402. The ligament 2502 itself and its movement through a motion of the joint may be simulated (e.g., based on available models, a statistical model, and/or prior procedure data 1050 from multiple patients). For example, the ligament 2502 may be based on a known model and located on a known area of a bone where an average ligament (e.g., average MCL) would be located. In some examples, ligament 2502 may be modeled using image analysis system 10 and/or based on patient's own anatomy (e.g., using patient data 1020 such as from previous surgeries or imaging data 1010 using a modality capable of imaging ligaments). The ligament 2502 may rotate and/or translate as the joints move through motion (e.g., flexion and extension).

The image analysis system 10 may correspond surfaces or features of a bone model to surfaces or features of the artificial model 402. For example, the artificial model 402 may include a same number of vertices, faces, triangles, bases, etc. as a patient bone or other statistical bone model. These features may move as a shape of the bone changes slightly. These features may define a set of points or locations in the artificial model 402 of a bone. The image analysis system 10 may create a mask over those points based on known positions. For example, the mask may include a ligament representation, and the mask may be overlaid onto the points or features based on a known location of the ligament (e.g., MCL, ACL, etc.). The mask may be displayed over the bone of the artificial model 402.

The twenty-first GUI 288 may simulate movement of both the ligament 2502 and joint so that a practitioner may assess how the ligament 2502 will interact with osteophytes (indicated by indicators 404) during movement. In some examples, the osteophytes and ligament 2502 displayed may represent a preoperative state. In other examples, the osteophytes and the ligament 2502 displayed may represent a predicted postoperative state based on a current procedure plan 2020 and/or planned procedure data 1030. In yet other examples, the osteophytes and ligament 2502 displayed may represent a predicted state if a patient does not undergo treatment. The twenty-first GUI 288 may allow the practitioner to evaluate the procedure plan 2020 and make modifications or adjustments based on assessment of the ligament 2502 with respect to the osteophytes during motion. As the twenty-first GUI 288 displays simulated movement, attachment points of the simulated ligament 2502 may remain the same. In some examples, the osteophytes and ligament 2502 displayed may represent an intraoperative state (e.g., during a procedure as potentially new and/or intraoperative data is received) and/or a postoperative state after a procedure using intraoperative and/or postoperative data. In yet other examples, the osteophytes and ligament 2502 displayed may represent a predicted long-term state after the procedure to allow a practitioner to assess a need for revision surgery and/or further treatment based on patient outcomes.

In some examples, the twenty-first GUI 288 may display a determined or predicted movement, rather than a simulation based on a statistical model or available model. For example, the image analysis system 10 may determine, via the one or more algorithms 90 (e.g., alignment/deformity algorithm 80) how the patient's anatomy currently moves, how the patient's anatomy would be predicted to move if the patient does not undergo treatment, how the patient's anatomy would be predicted to move if the patient undergoes treatment (e.g., the procedure plan 2020) and/or a desired or ideal movement. The image analysis system 10 may generate one or more simulations of the determined movement. For example, the image analysis system 10 may generate images of movement of a tibia and femur relative to each other throughout an entire range of motion of a knee joint. The display of osteophytes on the tibia and femur, and the osteophyte's relative position throughout a range of motion of a knee joint, may facilitate the identification of osteophytes that may hinder a patient's range of motion and/or cause pain during movement of the patient's knee joint.

The twenty-first GUI 288 may further display related metrics and/or determinations by image analysis system 10 corresponding to the simulated movement of the ligament 2502. For example, the image analysis system 10 may determine a perceived pain associated with the simulated movement of the ligament 2502, a measurement and/or size of a gap between the ligament 2502 and surrounding bone, a range of extension and/or stretch of the ligament 2502, an extent of a stretch and/or extent of the ligament 2502 beyond a predetermined threshold and/or average value, etc.

### Simulated Implant GUI

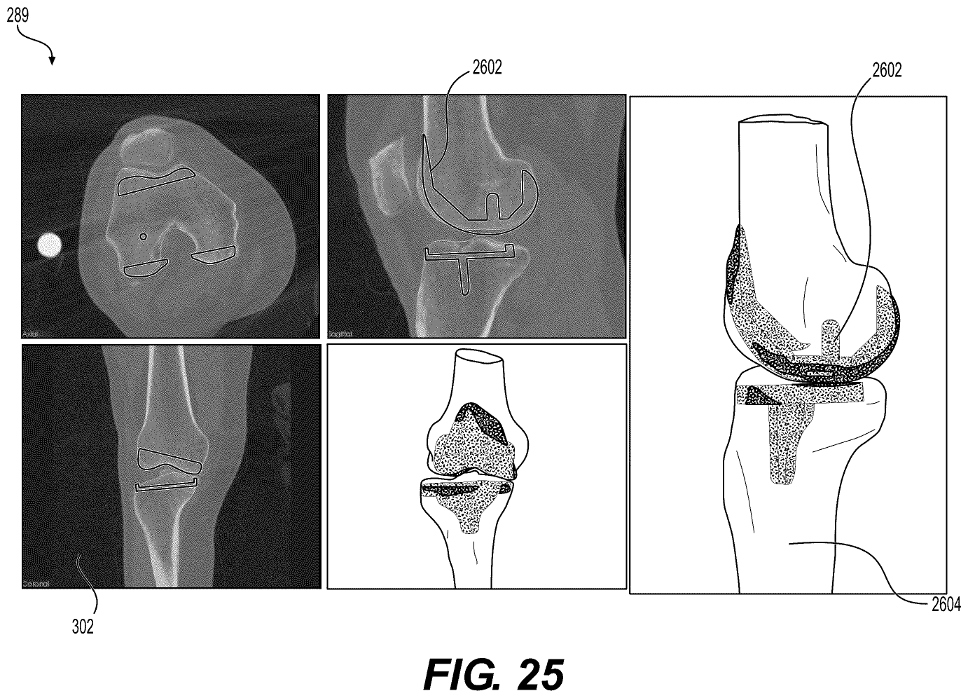

Referring to FIG. 25, the image analysis system 10 may determine, based parameters determined by the one or more algorithms 90, that the procedure plan 2020 should include a certain implant design or dimensions. For example, based on a determined joint-space width or joint-space narrowing by the joint-space width algorithm 50, the image analysis system 10 may determine that an implant width should be decreased and/or determine a type of implant (e.g., a constrained type) based on a narrower determined joint-space width or joint-space narrowing. Based on a joint width and/or an increased joint-space width determined by the joint-space width algorithm 50 and/or a looser or less stable joint determined by alignment/deformity algorithm 70, the image analysis system 10 may determine that an implant width should be increased (e.g., with augments or shims) and/or determine a type of implant should be a stabilizing or constrained type of implant, that a type or extent of procedure in the procedure plan 2020 should include a more corrective surgery, such as from a partial joint (e.g., knee, hip, or shoulder) replacement to a total joint replacement, etc.

As exemplified in FIG. 25, the image analysis system 10 may visually depict, in a twenty-second GUI 289, a determined implant design as a model implant 2602 overlaid or superimposed on an acquired image 302 (e.g., CT scan) and/or on representative model 2604. FIG. 25 shows an acquired image 302 of a knee and a model implant 2602 configured to be coupled to at least a portion of the knee.

### Bone Resection GUIs

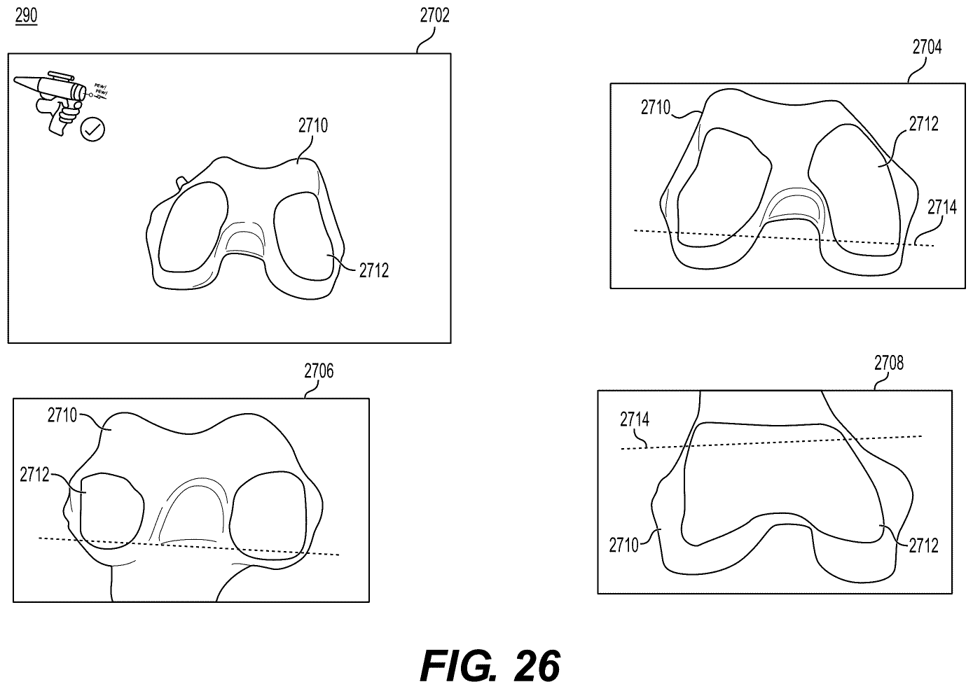

Aspects disclosed herein may be used to determine geometry and/or dimensions for bone cuts or resections and/or implant design. FIGS. 26 and 27 illustrate exemplary GUIs depicting bone resection planes and/or a virtual bone model.

Referring to FIG. 26, the one or more GUIs 250 may include a twenty-third or bone resection GUI 290 having at least one screen or frame 2702, 2704, 2706, and/or 2708. The at least one frame 2702, 2704, 2706, and/or 2708 may display a representative model determined by the image analysis system 10 and having at least one bone 2710 (e.g., tibia or femur).

The image analysis system 10 may determine, using the one or more algorithms 90 (e.g., osteophyte detection algorithm 60) a recommended or planned resection area or volume 2712 (for example, as part of procedure plan 2020). As an example, the image analysis system 10 may determine value for the resection area or volume 2712 based on a determined osteophyte volume, and may determine a location of the resection area or volume 2712 based on one or more detected osteophyte locations. The bone resection GUI 290 may display the determined resection area or volume 2712 overlaid on the at least one bone 2710. The at least one frame 2702, 2704, 2706, and/or 2708 may include a plurality of frames 2702, 2704, 2706, and/or 2708 showing various orientations and/or perspectives of the determined resection area or volume 2712 on the at least one bone 2710.

The image analysis system 10 may determine, using the one or more algorithms 90, a recommended or desired cut start line 2714 where a practitioner (e.g., surgeon) should position a surgical tool (e.g., burr or other cutting tool) to produce the displayed, determined resection area or volume 2712. The bone resection GUI 290 may display the recommended or desired cut start line 2714 overlaid on the at least one bone 2710 and/or the determined resection area or volume 2712. In some examples, the bone resection GUI 290 may determine multiple cut start lines 2714, which may be displayed in separate frames 2704, 2706, and/or 2708. In some examples, the bone resection GUI 290 may determine an updated or adjusted start line 2714 based on a progression of a procedure, a cut, or other newly received information.

Referring to FIG. 27, the one or more GUIs 250 may include a twenty-fourth or virtual bone GUI 292 having at least one screen or frame 2802, 2804, 2806, 2810, and/or 2812. The at least one frame 2802, 2804, 2806, 2810, and/or 2812 may display a representative model determined by the image analysis system 10 and having at least one bone 2814 (e.g., tibia or femur).

The image analysis system 10 may determine, using the one or more algorithms 90 (e.g., osteophyte detection algorithm 60) one or more bone cuts or planes 2816, 2818, 2820, 2822, 2824, 2826 (for example, according to procedure plan 2020). The virtual bone GUI 292 may display the determined bone cuts 2816, 2818, 2820, 2822, 2824, 2826, 2828, and 2830 overlaid on the at least one bone 2814. For example, at least one frame 2802, 2804, 2806, 2810, and/or 2812 may display a posterior cut 2816, a posterior chamfer cut 2818, a distal cut 2820, an anterior chamfer cut 2822, an anterior cut 2824, a floor cut 2826 (e.g., tibial floor cut), a peg cut 2828, and/or a wall cut 2830. The at least one frame 2802, 2804, 2806, 2810, and/or 2812 may include a plurality of frames frame 2802, 2804, 2806, 2810, and/or 2812 that display the bone 2814 in various orientations to best display the determined bone cuts 2816, 2818, 2820, 2822, 2824, 2826, 2828, and 2830. The image analysis system 10 may also determine, using the one or more algorithms 90, a desired or recommended implant design 2832, and the virtual bone GUI 292 may display the determined implant design 2832 (with or without bone 2814). The image analysis system 10 may determine certain planes or lines 2834 corresponding to a geometry of the bone 2814 and/or the determined bone cuts 2816, 2818, 2820, 2822, 2824, 2826, 2828, and 2830.

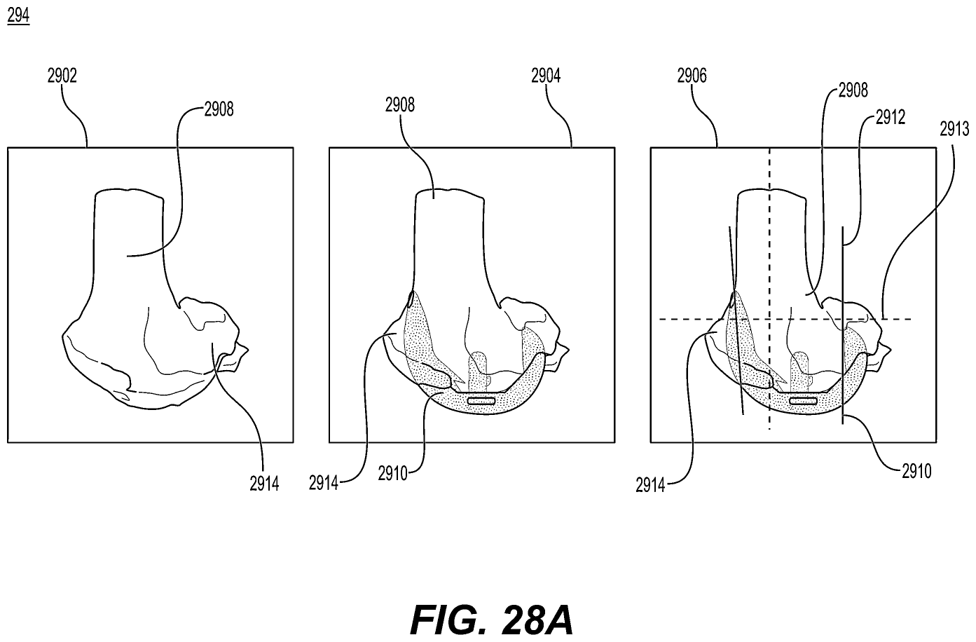

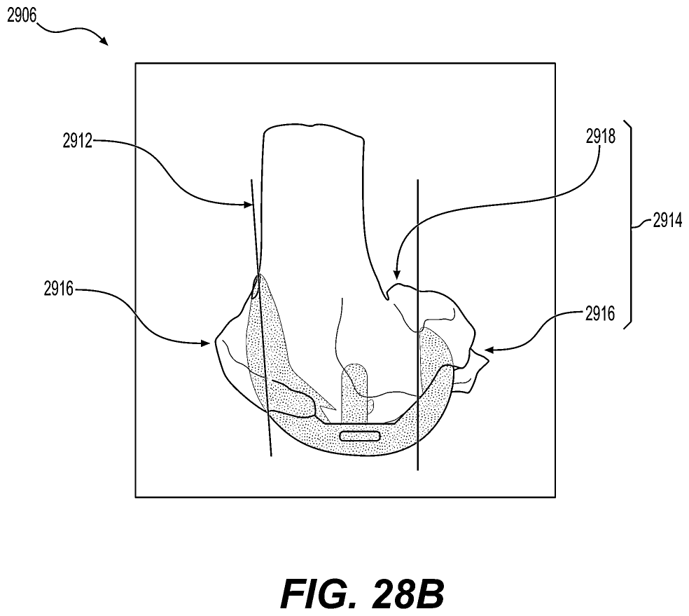

Referring to FIG. 28A, the one or more GUIs 250 may include a twenty-fifth GUI or a bone cut GUI 294 having at least one screen or frame 2902, 2904, and/or 2906. The at least one frame 2902, 2904, and/or 2906 may display a representative model determined by the image analysis system 10 and having at least one bone 2908 (e.g., tibia or femur) and implant 2910 (e.g., as determined by the image analysis system 10). The bone 2908 may include at least one osteophyte 2914. The bone 2908 and/or the osteophyte 2914 may be translucent or transparent so that the full implant 1910 may be visible.

The image analysis system 10 may determine, using the one or more algorithms 90 (e.g., osteophyte detection algorithm 60) one or more bone cuts 2912 (for example, according to procedure plan 2020). The bone cuts 2912 may be configured to remove the osteophytes 2914. The bone cut GUI 294 may display the determined bone cuts 2912 overlaid on the at least one bone 2908 and/or implant 2910.

For example, at least one frame 2902, 2904, and/or 2906 may include a first frame 2902, a second frame 2904, and a third frame 2906. The first frame 2902 may be configured to display the bone 2908 (e.g., femur) that has been segmented. The second frame 2904 may be configured to display the implant 2910 overlaid on the bone 2908 (e.g., according to procedure plan 2020). The third frame 2906 may be configured to display a planned or determined bone cut 2912 overlaid on the bone 2908 to show how much bone tissue and/or osteophytes 2914 would be removed using the bone cut 2912. For example, the third frame 2906 may display a view that shows a cross-section of a plane of a bone cut 2912. The third frame 2906 may also display the implant 2910 overlaid on the bone 2908. In some examples, the third frame 2906 may display a reference axis, plane, or grid 2913 relative to the bone cut 2912.

The image analysis system 10 may also determine, using the one or more algorithms 90, a desired or recommended implant design of the implant 2910, and the bone cut GUI 294 may display the determined implant design of the implant 2910 (with or without bone 2908).

FIG. 28B shows an enlarged view of the third frame 2906. Referring to FIG. 28B, the one or more osteophytes 2914 may include a first portion 2916 removed from the bone cut 2912 and a second portion 2918 that remain after the bone 2908 is cut according to the bone cut 2912. As shown in FIG. 28B, the first portion 2916 removed is displayed on an outer side of the bone cut 2912, while the second portion 2918 remaining is displayed on an inner side of the bone cut 2912.

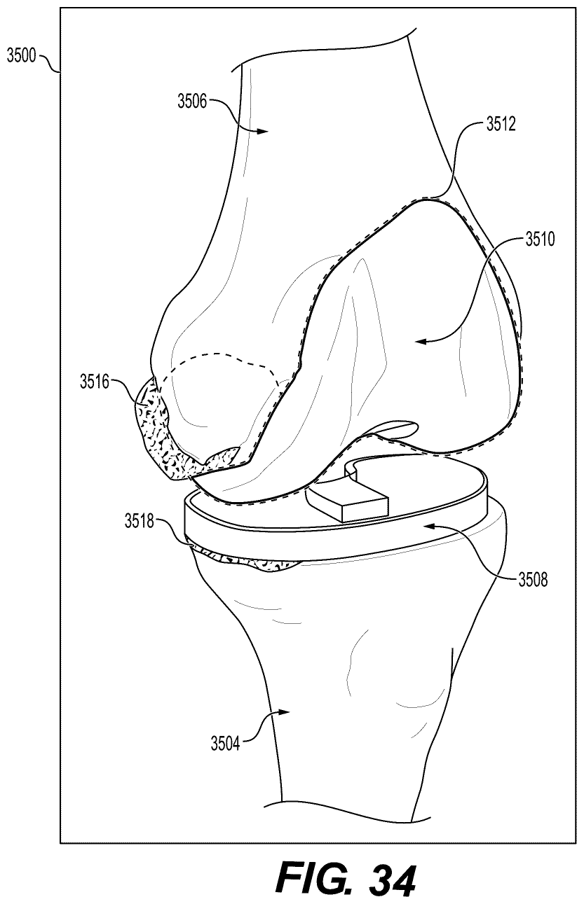

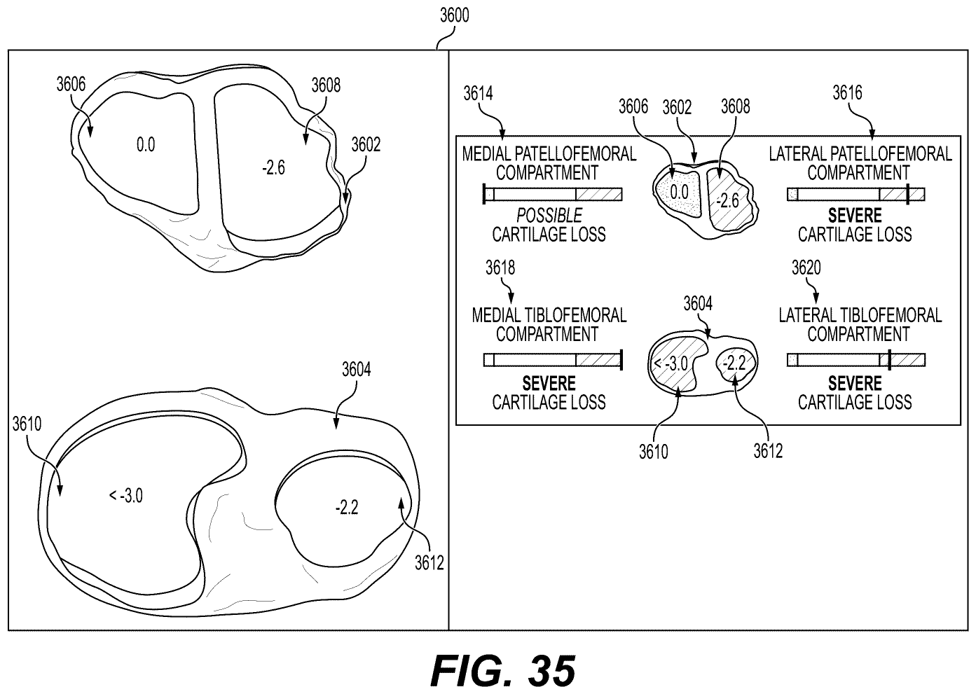

Referring to FIG. 34, a GUI 3500 that includes an exemplary post-operative model generated by the image analysis system 10 with a virtual tibia 3504 and femur 3506. Virtual models of implants 3508, 3510 are shown installed on the tibia 3504 and femur 3506. A resection line 3512 is shown overlaid onto the femur 3506, and resection line 3512 may indicate the cut path on the femur 3504 of a surgical plan. The bone resections, resection lines 3512, and implants 3508, 3510 may be determined by the image analysis system 10. The tibia 3504 and the femur 3506 may include at least one osteophyte 3516, 3518, and osteophytes 3516, 3518 may be determined by the image analysis system 10. In some examples, the bones 3504, 3506, a wireframe, and/or the osteophytes 3516, 3518 may be displayed as translucent or transparent so that the entirety of each implant 3508, 3510 may be visible. In some other examples, implants 3508, 3510 may be transparent or translucent to show other features, such as intended bone to be removed. The transparency and/or translucency may be turned on and off, depending on the needs of the user. In some examples, bones 3504, 3506 may be removed from the display upon a control command, and re-inserted into the display upon a second actuation of the control command. Implants 3508 is positioned over the proposed resection area of the tibia 3502, and implant 3510 is positioned over the proposed resection area of the femur 3506. In this example, display of resection line 3512 and surgical implant 3510 may facilitate determining how well the implant 3510 will fit after the cuts have been made with that particular surgical plan, and may allow a surgeon to adjust the resection line 3512 based on the relative positioning of resection line 3512 and implant 3510. The surgeon will be able to inspect the proposed cuts outlined by the resection line 3512 with reference to a implant 3510, and in some examples, may adjust the position of resection line 3512 using one or more controls of GUI 3500. For example, a user may select resection line 3512 and “drag and drop” the resection line 3512 to a new position on femur 3506. In some examples, the osteophytes 3516, 3518 on the tibia 3506 and femur 3504 are also cut so that the implants 3508, 3510 may be installed. Although not shown in this figure, the image analysis system 10 may create and display a resection line corresponding to the proposed resection of the femur as well. A user may be able to manipulate the position of tibia 3504, femur 3506, and implants 3508, 3510, to visualize resection line 3512 in three-dimensions and view resection line 3512 from any perspective. In some examples, multiple resection lines 3512 may be displayed on a bone, such as femur 3506 and/or tibia 3504, to allow a user to compare different potential resection lines 3512. In some examples, a user may actuate a control, such as a button or other control command for GUI 3500, to remove osteophytes 3516, 3518 from the display of GUI 3500.

### Bone Mineral Density (BMD) Algorithm and Graphic User Interfaces (GUIs)

Referring to FIGS. 2 and 43, the bone mineral density (BMD) algorithm 87 and/or the other one or more algorithms 90 may analyze imaging data 1010 to determine one or more outputs 2000 to display within a GUI (e.g., GUI 4200), such as a BMD of a patient's bone. BMD may refer to an amount of bone mineral present in the bone tissue of a bone, and may be factored into a variety of medical treatments and/or used as an indicator of various medical conditions, such as osteoporosis or osteopenia. For example, BMD may be used for bone health assessments, risk predictions, monitoring treatment and disease, and preoperative planning for surgeries such as knee and hip replacements, among others.

Referring to FIGS. 40A-40B and 41, BMD is often clinically measured by proxy through an analysis of medical images. However, a BMD typically may not be derived from a medical image directly, and may require additional processing and/or calibration. For example, in many instances, a BMD of a bone 4112 may be determined using an image generated by a computed tomography (CT) scan including the bone 4112 and a calibration device, such as a calibration phantom 4120. A calibration phantom 4120 may include a plurality of different reference materials (e.g., reference materials 4122, 4124, 4126) for each of which a BMD equivalent is known, and may be placed near the bone 4112 during the CT scan. The medical image 4102 generated by the CT scan represents the attenuation of x-rays as they pass through different materials, indicated by different intensities exhibited in the medical image 4102 (with higher intensities appearing lighter and lower intensities appearing darker, in this example). At least one reference material of the plurality of different reference materials included in the calibration phantom 4120 is configured to cause a different attenuation of the x-rays than the bone 4112, thereby producing a different intensity exhibited in the medical image 4102 than that of the bone, and because the BMD equivalent of the at least one reference material is known, the intensity of the at least one reference material and the BMD equivalent of the at least one reference material may be used as reference points to determine the BMD of the bone 4112. However, such calibration phantoms 4120 are often unavailable and/or uncomfortable for the patient. Thus, a method for determining a BMD of a bone from a medical image that is more accessible, reliable, accurate, and comfortable than using a calibration phantom is desired.

Referring to FIGS. 40A and 42, BMD algorithm 87 and/or the one or more algorithms 90 may be configured to determine a BMD of a patient's bone from an acquired image 302 using a BMD equivalent from only a single reference material included in a calibration device. For example, the calibration device may be a motion rod 4110. A motion rod 4110 may be a straight rod or bar that is physically coupled to a patient's anatomy or limb (e.g., the patient's leg) during a medical imaging process (e.g., a CT scan or an MRI scan) to hold the patient's anatomy still or straight. Additionally or alternatively, to ensure the accuracy of a medical imaging process, a motion rod 4110 may be used to determine if a patient's anatomy remained still or straight during the medical imaging process. For example, any motion of the motion rod 4110 detected during a medical imaging process may indicate that a patient's anatomy was moved during the medical imaging process, which may require a rejection or repeat of the medical imaging process. A motion rod 4110 may be included as a standard component of a medical imaging process protocol. A motion rod 4110 may be radiopaque, and may made of aluminum or any other suitable material.

Referring still to FIG. 42, in one example, the BMD algorithm 87 may be configured to determine a BMD of a patient's bone from an acquired image 302 using a BMD equivalent from only a single reference material included in a calibration device (e.g., a motion rod 4110) by first identifying, from within the acquired image 302, intensities of the single reference material included in the calibration device, a bone 4112 of the patient's anatomy, and at least one of fat 4114 from the patient's anatomy and air 4116. In this example, the BMD algorithm 87 then receives, accesses, retrieves, or otherwise obtains predetermined BMD equivalents for the single reference material included in the calibration device and the at least one of the fat 4114 and the air 4116. Then, using the intensities and the BMD equivalents of the single reference material included in the calibration device and the at least one of the fat 4114 and the air 4116, the BMD algorithm 87 determines a BMD calibration factor (BCF) for the acquired image 302. The BCF may be determined using a linear regression, a best fit line, or any other suitable method. Since both (i) the single reference material included in the calibration device and (ii) the at least one of the fat 4114 and the air 4116 may typically be present in a medical image, using the intensities and BMD equivalents of the single reference material included in the calibration device and the at least one of the fat 4114 and the air 4116 to determine the BCF may be more reliable than using a calibration phantom 4120 (as described above). Furthermore, the single reference material included in the calibration device and the at least one of the fat 4114 and the air 4116 may produce a wider range of intensities than a calibration phantom 4120, thereby producing a more accurate BCF. After determining the BCF, the BMD algorithm 87 may apply the BCF to the intensity (i.e. the CT image data) of the bone 4112 to determine the BMD of the bone 4112. The BMD of the bone 4112 may then be used to generate one or more GUIs. For example, the BMD of the bone 4112 may be used to generate and display an artificial or virtual (e.g., two-dimensional or three-dimensional) model of the patient's anatomy, exhibiting the BMD of the bone 4112, within a GUI.

In some instances, in which intensities of only one of fat 4114 and air 4116 is used to generate the BCF, the BMD algorithm 87 may be configured to compare the intensity and/or the BMD equivalent of the one of the fat 4114 and the air 4116 that was not used in determining the BCF to one or more relationships between the intensities and the BMD equivalents of the single reference material included in the calibration device and the one of the fat 4114 and the air 4116 that was used in determining the BCF, to determine if the BCF is acceptably accurate. For example, if the intensity and/or the BMD equivalent of the one of the fat 4114 and the air 4116 that was not used in determining the BCF does not fit, within an acceptable threshold, a linear regression or a best fit line generated using the intensities and the BMD equivalents of the single reference material included in the calibration device and the other of the fat 4114 and the air 4116, the BMD algorithm 87 may determine that the BCF is not acceptably accurate. In some instances, intensities and BMD equivalents of the single reference material included in the calibration device, the fat 4114, and the air 4116 are used to determine the BCF. In some instances, the bone 4112, the single reference material included in the calibration device, and the fat 4114 and/or the air 4116, and/or their respective intensities, may be identified from within the acquired image 302 using a shape model.

In some instances, instead of using a calibration device (e.g., a motion rod 4110) to determine a BMD of a patient's bone, the BMD algorithm 87 may use cortical bone as a reference material. For example, the BMD algorithm 87 may identify, from an acquired image 302, intensities of cortical bone from the patient's anatomy and at least one of fat 4114 from the patient's anatomy and air 4116. The BMD algorithm 87 may then receive, access, retrieve, or otherwise obtain BMD equivalents for cortical bone and the at least one of the fat 4114 and the air 4116. Then, using the intensities of cortical bone and the at least one of the fat 4114 and the air 4116, the BMD algorithm 87 may determine a BCF for the acquired image 302. Since both cortical bone, fat 4114, and air 4116 may typically be present in a medical image, using intensities and BMD equivalents of cortical bone, fat 4114, and/or air 4116 to determine a BCF for the medical image may be more reliable, feasible, or cost effective than using a calibration phantom 4120 (as described above) or a motion rod 4110 (as described above).

FIG. 43 illustrates a GUI 4200 provided or generated by, or otherwise communicatively coupled to the image analysis system 10, similar to the GUI 255 of FIG. 4E. GUI 4200 displays an artificial model 402 of a patient's anatomy (e.g., a patient's knee). In this example, in addition to the B-score widget 414 (as described above) and the C-score widget 416 (as described above), the GUI 4200 includes a bone mineral density (BMD) widget 4202. As illustrated in FIG. 43, the B-score widget 414 may display various information regarding a B-score determined for the patient (e.g., by B-score algorithm 70), such as an image of a bone that represents the B-score or a gradient bar that includes the B-score in the context of a scale, as described above. As illustrated in FIG. 43, the C-score widget 416 may display various information regarding a C-score determined for the patient (e.g., by joint-space width algorithm 50), such as an image of a bone that represents the C-score or a gradient bar that indicates the C-score in the context of a scale, as described above. Similarly, as illustrated in FIG. 43, the BMD widget 4204 displays various information regarding a BMD determined for the patient, such as a gradient bar that indicates the BMD in the context of a scale. The GUI 4200 may allow a user of the GUI 4200 (e.g., a surgeon or a medical practitioner) to select any point or area on the artificial model 402 of the patient's anatomy and view a BMD determined for a corresponding point or area of the patient's anatomy within the BMD widget 4202. If the user selects an area of the BUI 4200, the BMD determined may correspond to an average BMD across the selected area.

### Other Outputs 2000

Referring back to FIG. 2, the one or more algorithms 90 may also determine (or be used by the image analysis system 10 to determine) other outputs 2000 to display on the one or more GUIs 250 such as aspects of the procedure plan 2020 including steps, instructions, tools, etc. for preparing for and/or performing a procedure (e.g., surgery). The procedure plan 2020 may include a planned number, position, length, slope, angle, orientation, etc. of one or more tissue incisions or bone cuts, a planned type of the implant, a planned design (e.g., shape and material) of the implant, a planned or target position or alignment of the implant, a planned or target fit or tightness of the implant (e.g., based on gaps and/or ligament balance), a desired outcome (e.g., alignment of joints or bones, bone slopes such as tibial slopes, activity levels, or desired values for postoperative outputs 2000), a list of steps for the surgeon to perform, a list of tools that may be used, etc. The image analysis system 10 may determine that a type or extent of the procedure in the procedure plan 2020 should include a more corrective surgery, such as from a partial joint (e.g., knee, hip, or shoulder) replacement to a total joint replacement, that certain fixation or other techniques should be used, whether cementing techniques or cementless techniques or implants should be used, etc.

The procedure plan 2020 may, for example, include instructions on how to prepare a proximal end of a tibia to receive a tibial implant, how to prepare a distal end of a femur to receive a femoral implant, how to prepare a glenoid or humerus to receive a glenoid sphere and/or humeral prosthetic component, how to prepare a socket area or acetabulum to receive a ball joint, etc. The bone surface may be cut, drilled, or shaved relative to a reference (e.g., a transepicondylar axis). The procedure plan 2020 may include positions, lengths, and other dimensions for the surfaces and/or values for the slopes for bone preparation. As will be described later, the procedure plan 2020 may be updated and/or modified based on intraoperative data 3000. The one or more GUIs 250 may include a GUI configured to display the procedure plan 2020 and/or related steps.

The procedure plan 2020 may also include predictive or target outcomes and/or parameters, such as target postoperative range of motion and alignment parameters, and target scores (e.g., stability, fall risk, joint stiffness or laxity, or OA progression). The one or more GUIs 250 may include a GUI configured to display these target and/or predicted parameters. These target parameters may ultimately be compared postoperatively to corresponding measured postoperative data or results to determine whether an optimized outcome for a patient was achieved. The image analysis system 10 may be configured to update the procedure plan 2020 based on manual input and/or feedback input by practitioners, newly acquired preoperative data 1000, or patient feedback.

The image analysis system 10 may also determine, assign, and/or designate assigned staff 2050 to assist in performance of the procedure. For example, the image analysis system 10 may determine that the assigned staff 2050 should include surgeons, nurses, or other individuals having more experience with a type of surgery (e.g., knee surgery or total knee arthroplasty) planned in the procedure plan 2020 and/or having more experience with patients having similar characteristics as the instant patient (e.g., narrower joint space width, patient history, a certain type of deformity etc.) The image analysis system 10 may determine that the assigned staff 2050 should include surgeons, nurses, or other individuals having experience with procedures that take as long as the predicted procedure time 2010. The image analysis system 10 may store or determine experience scores or levels for each staff member, and may determine an average of a composite procedure or staff team and/or use a rolling average to determine the assigned staff 2050.

The image analysis system 10 may determine that the assigned staff 2050 should have, individually and/or collectively, more experience based on: a certain type or more complex implant plan, a narrower (or narrowing over time) joint space width determined by the joint space width algorithm 50, a larger osteophyte volume or osteophyte number (or increasing osteophyte volume or number over time, or an osteophyte volume outside of a predetermined range) determined by the osteophyte detection algorithm 60, a higher (or increasing) B-score determined by the B-score algorithm 70, a severe or complicated deformity detected by the alignment/deformity algorithm 80, an OA progression determined using the one or more algorithms 90, impingement data calculated using parameters determined from the joint space width algorithm 50, the osteophyte detection algorithm 60, and/or the alignment/deformity algorithm 80, etc. The one or more GUIs 250 may include a GUI configured to display the assigned staff 2050.

The image analysis system 10 may also determine an operating room layout 2030 and an operating room schedule 2040 based on joint-space width parameters determined by the joint-space width algorithm 50, osteophyte volume parameters determined by the osteophyte detection algorithm 60, B-score determined by the B-score algorithm 70, a bone-to-tissue ratio, PPT, and/or PTT, and/or based on the predicted procedure time 2010 or other determinations or outputs 2000 (e.g., assigned staff 2050). The OR layout 2030 may include a room size, a setup, an orientation, starting location, positions and/or a movement or movement path of certain objects or personnel such as robotic device 142, a practitioner, surgeon or other staff member, operating room table, cameras, displays 210, other equipment, sensors, or patient. The image analysis system 10 may determine a series of alerts, warnings, and/or reminders sent to practitioners, hospital staff, and/or patients in preparation for the operation and/or during the operation. The image analysis system 10 may determine or output a new alert to practitioners, hospital staff, and/or patients based on a change in any of the previously determined outputs 2000, which may be based on newly acquired preoperative data 1000 and/or intraoperative data 3000 described later. In some examples, an alert may be a message or indication displayed on a graphical user interface preoperatively or intraoperatively. The one or more GUIs 250 may include a GUI configured to display the OR layout 2030.

The image analysis system 10 may also determine or be used to determine surgeon ergonomics 2070 guidance. For example, the image analysis system 10 may recommend certain postures or positions for assigned staff 2050 based on a longer predicted procedure time 2010 (and/or parameters associated with a longer procedure time 2010, such as a narrower joint space width, a larger osteophyte volume, a larger B-score, a more severe deformity, a larger bone-to-tissue ratio, PPT, and/or PTT, etc.), past experience of the assigned staff 2050, and/or tools to use as part of the determined procedure plan 2020. The image analysis system 10 may optimize surgeon ergonomics 2070 to reduce and/or optimize the predicted procedure time 2010. The one or more GUIs 250 may include a GUI configured to display steps or recommendations based on the determined surgeon ergonomics 2070.

Referring to FIG. 36, the image analysis system 10 may determine a density of the tibia 3704 and the femur 3702 and display it on a GUI 3700. Shown in FIG. 36, the virtual model of the tibia 3704 is displayed with apportions of the tibia 3704 marked with shading and/or outlined to show two bone density display portions 3706, 3708. In some examples, the bone density display portions 3706, 3708 may be shown centered below one or more contact points of the femoral condyles with the tibia 3704 when a leg is in extension and/or may be centered below the deepest point of the medial and lateral tibia surface where the load/pressures are the highest. Although the bone density display portions 3706, 3708 are shown positioned in this manner, a user may have the ability to adjust the positioning of bone density display portions 3706, 3708 to anywhere within any of the displayed bones, may adjust the size of bone density display portions 3706, 3708, and may adjust the number of bone density display portions 3706, 3708. Or for example, when determining and/or displaying bone density display portions, the image analysis system 10 may extract and/or remove the cortical bone from the tibia 3704 and/or the femur 3702. The bone density display portions may then be displayed over the trabecular or cancellous bone of the tibia 3704 and/or the femur 3702. In some examples, the cancellous bone of the tibia 3704 or any other bone may be spit into lateral and medial epiphyseal and/or metaphyseal areas, and such areas may be displayed along with the associated bone density and/or other parameters described herein. The 3D image model 3710, including the bone density display portions 3706, 3708, may determine an exact depth, width, height, and location of the bone density display portions 3706, 3708. In other examples, these dimensions may be user selected, and/or a user may adjust the determined depth, width, height, and location of the bone density display portions 3706, 3708. The location, size, and/or exterior surface angles of the bone density display portions 3706, 3708 may be determined using the planned tibial cut(s) (e.g., 3 mm below the original tibial surface). The volume may have fixed dimensions (e.g., approximately the size of a human thumb). Typically, a surgeon may test a resected area to assess bone stability/strength of the tibia surface after making the tibial cut and feel with their thumb whether the cut bone is soft, compressible, and/or spongeous. When the bone is soft, compressible, and/or spongeous, the surgeon may adjust the surgical plan to cut more of the tibia or choose a cemented implant for stability. In this example shown in FIG. 36, the 3D model 3710 of the target bone 3704 may be rendered to display the bone density display portions 3706, 3708 of the user or system specified (e.g., specified by the image analysis system 10) bone portion (e.g., a portion of tibia 3704), which displays to a user a density gradient of the bone density display portions 3706, 3708, and showing the surgeon whether there are soft or spongeous areas within the bone before making a cut.

An average density value for the bone density display portions 3706, 3708 may be determined, along with a density threshold determined based on prior patient data or user defined. FIG. 36 illustrates the GUI 3700 with bone density display portions 3706, 3708 that are positioned within, or an interior portion of, bone 3704. However, in some examples, the density of the bone density display portions 3706, 3708 may be displayed next to the bone density display portions 3706, 3708, for example may be shown outside of bone density display portions 3706, 3708 and/or outside of bone 3704. In some examples, there may be a plot of maximum and minimum density in the bone density display portions 3706, 3708 to give the surgeon an indication of the density distribution within bone density display portions 3706, 3708, and this may be displayed separate from each bone density display portions 3706, 3708 and appropriately labeled with each associated bone density display portion 3706, 3708. In some examples, a cross section of the model 3710 may be created to allow the surgeon to view the density changes in a planar section through the bone density display portions 3706, 3708. In some examples, the user may define two different areas of the bone 3702, 3704 to compare differences in density measurement, and the density measurements may be displayed for each defined area to allow the user to quickly compare the measurements.

The GUI 3700 displaying the bone density display portions 3706, 3708 may be based on a direct volume rendering. The image analysis system 10 may determine a threshold density of the bone and display portions of the target bone (e.g., bone density display portions 3706, 3708) as transparent that have a lower density than the threshold. With portions of the target bone that are more dense than the threshold density, the higher density portions of the target bone will be displayed with a visual indicator such as a color, a pattern, a combination thereof, or the like (e.g., shade of gray or color map) based on the determined density value from the image analysis system 10. In this example, tibia 3704 is shown as transparent and the bone density display portions 3706, 3708 are indicated as a pattern. The 3D image generated by the image analysis system 10 of the target bone (e.g., tibia 3704; femur 3702) may then be rendered using a display that is repositioned by the surgeon or other user. In some examples, the display may be a surgical monitor 210. In other examples, the display may be an augmented reality display on a monitor 210 of the procedure system 240. In some other examples, the display may be a mobile device 220, such as a cell phone, a tablet, or other type of portable display. A color map may be included on the display to indicate hard and soft bone based on the defined threshold density. The color map may use a color gradient to indicate the hard and soft areas of the bone density display portions 3706, 3708. In some examples, after the image analysis system 10 has rendered the 3D image 3710 with the bone density display portions 3706, 3708, a Finite Element model may be generated so that another numeric value color map may be applied to the 3D model 3710.

With continued reference to FIG. 36, each of the bone density display portions 3706, 3708 may include a numeric value associated with the bone density. In some examples, the numeric value may be displayed within the bone density display portions 3706, 3708. In some examples, in addition to the numerical value associated with the bone density, or alternatively to the numerical value associated with the bone density, the numerical value associated with the bone density may be converted into a percentile relative to the bone densities of a group of other patients, for example of a group of prior patients and/or other patients with one or more similar attributes (e.g., gender, BMI, weight, injury location/surgery required, and/or age). In other examples, an average density across each bone density display portion 3706, 3708 may be displayed with the respective bone density display portions 3706, 3708 as determined by the image analysis system 10. In some other examples, the numeric values may be displayed next to the 3D model 3710 on the GUI 3700. In some examples, the bone density display portions 3706, 3708 may include a display of the estimated weight tolerance of the bone density display portion 3706, 3708 before compression or collapse.

### Intraoperative Systems

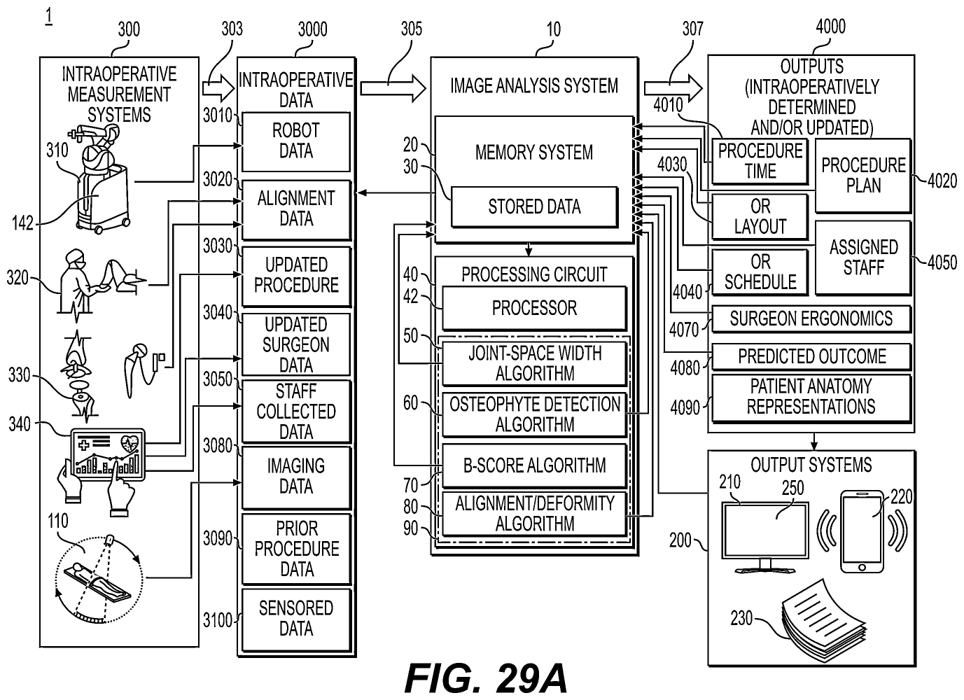

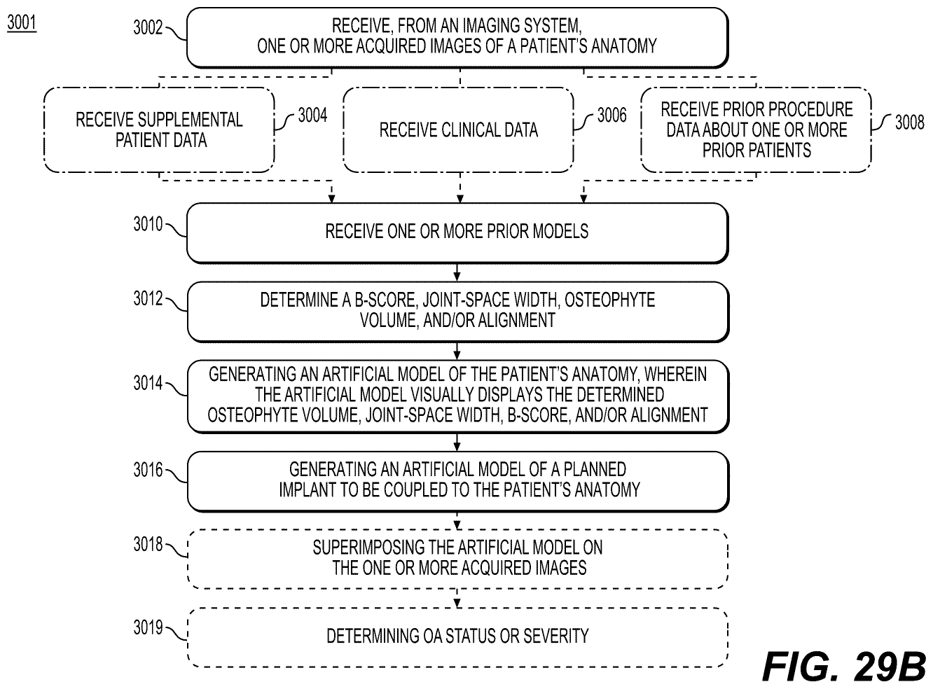

Referring to FIG. 29A, one or more intraoperative measurement systems 300 may collect (via arrow 303) intraoperative data 3000 during the procedure. During a medical treatment plan or procedure, the image analysis system 10 may collect, receive (e.g., from intraoperative measurement systems 300 via arrow 305), and/or store intraoperative data 3000. The image analysis system 10 may determine intraoperative outputs 4000 and output or send (via arrow 307) the intraoperative outputs 4000 to the output systems 200.

Although the term “intraoperative” is used, the word “operative” should not be interpreted as requiring a surgical operation. Postoperative data may also be collected, received, and/or stored after completion of the medical treatment or medical procedure to become prior procedure data 1050 for a subsequent procedure and/or so that the one or more algorithms 90 may be refined. The intraoperative outputs 4000 may be an updated or refined form of outputs 2000 determined preoperatively (FIG. 2) and/or may be newly generated. The intraoperatively determined outputs 4000 may also be referred to as secondary outputs 4000. Because many of the devices in the one or more intraoperative measurement systems 300 are similar to devices in the one or more preoperative measurement systems 100, many of the types of intraoperative data 3000 are similar to the preoperative data 2000, and many of the processes used and information included in the intraoperative outputs 4000 are similar to those with respect to the preoperatively determined outputs 2000. Any of the preoperative measurement systems 100 and data described herein may also be used and/or collected intraoperatively. Although certain information is described in this specification as being intraoperative data 3000 or intraoperatively determined outputs 4000 and/or postoperative data or postoperatively determined outputs, due to continuous feedback loops of data (which may be anchored by memory system 20), the intraoperative data 3000 described herein may alternatively be determinations or outputs 4000, and the intraoperatively determined outputs 4000 described herein may also be used as inputs into the image analysis system 10. For example, some intraoperative data 3000 may be directly sensed or otherwise received, and other intraoperative data 3000 may be determined, processed, or output based on other intraoperative data 3000, preoperative data 1000, and/or stored data 30.

Like the preoperative measurement systems 100, the intraoperative measurement systems 300 may include electronic medical records and/or user interfaces or applications 340 and imaging devices 350 (e.g., an intraoperative X-ray device or a fluoroscopy device configured for intraoperative use). The intraoperative measurement systems 300 may also include a robot system 310 including a robotic device 142 (e.g., surgical robot), sensors and/or devices 320 to conduct intraoperative tests (e.g., range of motion tests), and sensored implants 330 (e.g., a trial implant). The intraoperatively determined outputs 4000 may include intraoperatively determined (e.g., updated) or secondary procedure time or duration 4010, procedure plan 4020, OR layout 4030, OR schedule 4040, assigned staff 4050, surgeon ergonomics 4070, and/or predicted outcomes 4080.

The user interfaces or applications 340 may be used to input or update procedure information 3030, surgeon data 3040, and staff collected data 3050 (e.g., observations during a procedure and/or other data from sensors that may not have wireless communication modules, such as traditional thermometers). The updated procedure information 3030, surgeon data 3040, and staff collected data 3050 may be updated or refinements to preoperative data 1000 and/or newly generated. The imaging devices 350 may collect imaging data 3080, which may be similar to preoperatively collected imaging data 1010.

The robotic device 142 may be a surgical robot, a robotic tool manipulated or held by the surgeon and/or surgical robot, or other devices configured to facilitate performance of at least a portion of a surgical procedure, such as a joint replacement procedure involving installation of an implant. In some examples, a surgical robot may be configured to automatically perform one or more steps of a procedure. Robotic device refers to surgical robot systems and/or robotic tool systems, and is not limited to a mobile or movable surgical robot. For example, robotic device may refer to a handheld robotic cutting tool, jig, burr, etc.

For convenience of description, the robotic device 142 will be described as a robot configured to move in an operating room and assist staff in performing at least some of the steps of the preoperatively determined procedure plan 2020 and/or a newly generated, refined, or updated procedure plan 4040 (hereinafter referred to as “intraoperatively determined procedure plan 4040”).

The robotic device 142 may include or be configured to hold (e.g., via a robotic arm), move, and/or manipulate surgical tools and/or robotic tools such as cutting devices or blades, jigs, burrs, scalpels, scissors, knives, implants, prosthetics, etc. The robotic device 142 may be configured to move a robotic arm, cut tissue, cut bone, prepare tissue or bone for surgery, and/or be guided by a practitioner via the robotic arm to execute the procedure plan 2020 and/or intraoperatively determined procedure plan 4040. The determined procedure plan 2020 and/or intraoperatively determined procedure plan 4040 may include instructions and/or algorithms for the robotic device 142 to execute.

The robotic device 142 may include and/or use various sensors (pressure sensors, temperature sensors, load sensors, strain gauge sensors, force sensors, weight sensors, current sensors, voltage sensors, position sensors, IMUs, accelerometers, gyroscopes, position sensors, optical sensors, light sensors, ultrasonic sensors, acoustic sensors, infrared or IR sensors, cameras, etc.), sensored tools, cameras, or other sensors (e.g., timer, temperature, etc.) to record and/or collect robot data 3010.

The robot system 310 and/or robotic device 142 may include one or more wheels to move in an operating room, and may include one or more motors configured to spin the wheels and also manipulate surgical limbs (e.g., robotic arm, robotic hand, etc.) to manipulate surgical or robotic tools or sensors. The robotic device 142 may be a Mako SmartRobotics™ surgical robot, a ROBODOC® surgical robot, etc. However, aspects disclosed herein are not limited to mobile robotic devices 142.

The robotic device 142 may be controlled automatically and/or manually (e.g., via a remote control or physical movement of the robotic device 142 or robotic arm by a practitioner). For example, the procedure plan 2020 and/or intraoperatively determined procedure plan 4040 may include instructions that a processor, computer, etc. of the robotic device 142 is configured to execute. The robotic device 142 may use machine vision (MV) technology for process control and/or guidance. The robotic device 142 may have one or more communication modules (WiFi module, BlueTooth module, NFC, etc.) and may receive updates to the procedure plan 2020 and/or intraoperatively determined procedure plan 4040. Alternatively or in addition thereto, the robotic device 142 may be configured to update the procedure plan 2020 and/or generate a new and/or intraoperatively determined procedure plan 4040 for execution.

The robot data 3010 may include data relating to the operating room, movement by staff and/or the robotic device 142, actual time spent on steps of the procedure plan 2020 and/or intraoperatively determined procedure plan 4040, actual total procedure time (e.g., as compared to the determined procedure time 2010). The robotic system 310, via robotic device 142, may also collect or sense information regarding performed procedure steps, such as incision length or depth, bone cut or resection depth, or implant position or alignment. The robotic system 310, via robotic device 142, may also collect or sense information from the patient, such as biometrics pressure, body temperature, heart rate or pulse, blood pressure, breathing information, etc. The robotic system 310 may monitor and/or store information collected using the robotic device 142, and may transmit some of the information after the procedure is finished rather than during the procedure.

The other sensors and/or devices 320 may include one or more sensored surgical tools (e.g., a sensored marker), wearable tools, sensors, or pads, etc. The sensors and/or devices 320 may be applied to or be worn by the patient during the execution of procedure plan 2020 and/or intraoperatively determined procedure plan 4040, such as a wearable sensor, a surgical marker, a temporary surgical implant, etc. Although some sensors and/or devices 320 may also be sensored implants 330 or robotic devices 142 (e.g., robotic surgical tools configured to execute instructions and/or use feedback from sensors using motorized tool heads), other sensors and/or devices 320 may not strictly be considered an implant or a robotic device. For example, the sensors and/or devices 320 may be or include a tool (e.g., probe, knife, burr, etc.) used by medical personnel and including one or more optical sensors, load sensors, load cells, strain gauge sensors, weight sensors, force sensors, temperature sensors, pressure sensors, etc.

The image analysis system 10 may use the sensors and/or devices 320 to collect sensored data 3100, which may include pressure, incision length and/or position, soft tissue integrity, biometrics, etc. In addition, the sensored data 3100 may include alignment data 3020, range of motion data (e.g., collected during intraoperative range of motion tests by a practitioner manipulating movement at or about the joints) and/or kinematics data.

The one or more sensored implants 320 may include temporary or trial implants applied during the procedure and removed from the patient later during the procedure and/or permanent implants configured to remain for postoperative use. The one or more sensored implants 320 may include implant systems for a knee (e.g., femoral and tibial implant having a tibial stem, sensors configured to be embedded in a tibia and/or femur), hip (e.g., femoral implant having a femoral head having an acetabular component and/or stem), shoulder (e.g., humeral or humerus implant), spine (e.g., spinal rod or spinal screws), or other joint or extremities implants, replacements, prosthetics (e.g., fingers, forearms, etc.). The sensored implants 320 may include one or more load sensors, load cells, force sensors, weight sensors, current sensors, voltage sensors, position sensors, IMUs, accelerometers, gyroscopes, optical sensors, light sensors, ultrasonic sensors, acoustic sensors, infrared or IR sensors, cameras, pressure sensors, temperature sensors, etc.

The sensored implants 320 may collect sensored data 3100 and/or alignment data 3020, such as range of motion, pressure, biometrics, implant position or alignment, implant type, design, or material, etc. The sensored implants 320 may also be configured to sense and/or monitor infection information (e.g., by sensing synovial fluid color or temperature).

The intraoperative measurement systems 300 is not limited to the sensors discussed herein. For example, intraoperative data 3000 may also be collected using cameras or motion sensors installed in an operating room (e.g., camera above an operating table, high up on a wall, or on a ceiling) or a sensored patient bed or operating table (e.g., having temperature sensors, load cells, pressure sensors, position sensors, accelerometers, IMUs, timers, clocks, etc. to collect information on an orientation or position of the patient and biometrics, heart rate, breathing rate, skin temperature, skin moisture, pressure exerted on the patient's skin, patient movement/activity, etc., movement or position of the bed or table via wheel sensors, and/or a duration of the procedure). In addition, the intraoperative data 3000 may include prior procedure data 3090 from prior procedures with similar patients and/or similar intraoperative data 3000. The intraoperative data 3000 may include the same types of data in preoperative data 1000 and/or data such as operating room efficiency and/or performance, tourniquet time, blood loss, biometrics, incision length, resection depth, soft tissue integrity, pressure, range of motion or other kinematics, implant position or alignment, and implant type or design, though this list is not exhaustive.

As another example, cameras and/or a navigational system may be used to track operating room efficiency, pacing, layout information, information on staff and/or surgeon's performing the procedure plan 2020 and/or intraoperatively determined procedure plan 4020, and/or movement and posture patterns (measured by, for example, wearable sensors, external sensors, cameras and/or navigational systems, surgical robot 142, etc.). Based on intraoperatively collected data 3000, the image analysis system 10 may determine, in determining surgeon ergonomics 4070, that a table is too high for a surgeon and determine a lower height for the table in an updated operating room layout 4030, which may increase operating room efficiency and thus decrease a determined procedure duration 4010 and may reduce fatigue for a surgeon working over the operating table.

The image analysis system 10 may execute the one or more algorithms 90 to determine intraoperative outputs 4000 based on the intraoperative data 3000 similarly to how the one or more algorithms determined outputs 2000 based on the preoperative data 1000. The one or more algorithms 90 may also determine the intraoperative outputs 4000 based on the previously collected and/or stored intraoperative data 1000 and any other stored data 30, such as prior procedure data 3090. For example, the joint-space width algorithm 50 may use intraoperative data 3000 to determine, intraoperatively, joint space width dimensions, such as an updated joint space width between two bones based on intraoperative data 3000 and/or a new joint space width when an implant (e.g., trail implant 330 and/or permanent implant 330) is applied or other corrective steps in the procedure are performed. The osteophyte detection algorithm 60 may determine osteophyte position, dimension, and volume, such as an updated position and volume based on intraoperative data 3000 and/or a new position and volume after certain steps in the procedure are performed, such as when bone cuts are made. The B-score algorithm 70 may determine an updated B-score based on intraoperative data 3000 and/or a new B-score based on when an implant is applied or when other corrective steps in the procedure are performed. The alignment/deformity algorithm 80 may determine updated alignment and deformity information of the patient's bones based on intraoperative data 3000 and/or new alignment and deformity information after an implant is applied or certain steps of the procedure are performed.

Like outputs 2000 determined preoperatively, the intraoperative outputs 4000 may include surgical time 4010, procedure plan 4020, operating room layout 4030, operating room schedule 4040, assigned staff 4050, surgeon ergonomics 4070, predicted outcomes 4080, and patient anatomy representations 4090. As an example, based on complications during the procedure or due to certain information (e.g., alignment, deformity, or infection) that is more readily apparent intraoperatively once a tissue cut has been made, the image analysis system 10 may determine, intraoperatively, a new implant design as part of the procedure plan 4020 and/or new predicted outcomes 4080 (e.g., higher or lower risks or likelihoods for postoperative infection, perceived pain, stress level, anxiety level, mental health status, cartilage loss, and/or increase in the case difficulty). The image analysis system 10 may update the one or more GUIs 250 to account for a new implant model based on the newly determined implant design and/or new predicted outcomes 4080. These intraoperative outputs 4000 may be output on the previously described output systems 200.

As another example, the image analysis system 10 may determine that the procedure plan 4020 should include adjusted or extra steps, that an operating room layout 4030 should be adjusted, that the operating room schedule 4040 should be adjusted (and/or that other bookings using some same staff members or a same room should be adjusted) that the assigned staff 4050 should include more or less staff members, and/or that surgeon ergonomics 4070 should include positions suited to the longer duration.

In some cases, the image analysis system 10 may determine that the procedure should be stopped and/or postponed for a later date based on extreme complications of a patient's alignment and/or infection status and/or external factors (e.g., other emergencies at an institution, weather emergencies, etc.).

The intraoperative measurement systems 300 may periodically and/or continuously sense or collect intraoperative data 3000 (arrow 303), some or all of which may be periodically and/or continuously sent to the procedure time prediction system (arrow 305). The image analysis system 10 may periodically or continuously determine the intraoperatively determined outputs 4000 to update information and may periodically and/or continuously send the intraoperatively determined outputs 4000 to the output systems (arrow 307).

The image analysis system 10 may periodically and/or continuously compare the predicted outcome data 4080 with target or desired outcomes, and further determine, update, or refine the procedure duration 4010, the procedure plan 4020, and/or other outputs 4000 (e.g., OR layout 4030, OR schedule 4040, assigned staff 4050, and surgeon ergonomics 4070) based on the comparison. The image analysis system 10 may be configured to output this comparison (e.g., via information and/or visually) to the output system 200, such as the one or more GUIs 250 of the displays 210.

### Methods

Referring to FIGS. 2, 3, and 29B, an exemplary method 3001 according to an embodiment may be used to determine and/or generate one or more GUIs. The method 3001 may be merely exemplary and is not comprehensive of all aspects disclosed herein. The method 3001 may include a step 3002 of receiving, from an imaging system having an imaging device 110, one or more acquired images 302 of a patient's anatomy (e.g., leg or knee joint). The imaging device 110 may be a CT imaging machine, an MRI machine, an x-ray machine, etc. and the acquired image 302 may be a CT scan, an MR scan, an x-ray image, etc. The acquired image 302 may visualize internal structures (e.g., bone and/or tissues) of the instant patient. In step 3002, the image analysis system 10 may receive the acquired image 302 into memory system 20.

The method 3001 may also include a step 3004 of receiving patient specific data about the instant patient. The patient specific data may include patient data and medical history 1020. For example, the step 3004 may include receiving information about patient demographics, biometrics, treatment history, observations, etc. from EMR 120 and/or input (e.g., at an intake appointment) by a practitioner through an interface 130. Step 3004 may also include receiving patient information directly from the instant patient using, for example, an application through an interface 130 on a mobile device. In step 3004, the image analysis system 10 may store the patient specific data in memory system 20.

The method 3001 may also include a step 3006 of receiving clinical data, such as information about the planned procedure 1030 and/or surgeon or staff data 1040. The clinical data may be input by a practitioner or other staff into a user interface or application 130 to be received by the image analysis system 10. In step 3006, the image analysis system 10 may receive the clinical data into memory system 20.

The method 3001 may include a step 3008 of receiving prior procedure data 1050 of one or more prior patients. The prior procedure data 1050 may be input by a practitioner and received in memory system 20, or may already be incorporated into the stored data 30 of the memory system 20. The prior patients may share at least one physical characteristic (e.g., demographics, biometrics, disease or disease state, etc.) with the instant patient and may have undergone a similar procedure as the instant patient.

The method 3001 may include a step 3010 of determining, receiving, and/or selecting one or more prior models. The one or more prior models may be standard models or models obtained from healthy patients that represent a same anatomy type (e.g., leg or knee joint) as shown in the acquired images 302. Step 3010 may include recognizing one or more bone landmarks in the one or more received images, and determining a model that includes the recognized bone landmarks. Determining the prior model may also be based on received supplemental patient data, received clinical data, and/or received prior procedure data.

The method 3001 may include a step 3012 of determining at least one of a B-score, joint-space width, osteophyte volume, and/or alignment or deformity data based on the acquired image 302. In step 3012, the image analysis system 10 may use one or more algorithms 90 to determine parameters relating to B-score, joint-space width, osteophyte volume, and/or alignment or deformity for at least one bone of interest. For example, the image analysis system 10 may execute a B-score algorithm 70 to determine B-score and related parameters for a femur, a joint-space width algorithm 50 to determine a medial and/or lateral joint-space width between a femur and a tibia, an osteophyte detection algorithm 60 to determine a total osteophyte volume and/or number of osteophytes detected on the femur and tibia, and an alignment/deformity algorithm 80 to determine or detect alignment and/or deformities at the knee joint (e.g., a varus-valgus deformity and/or tilt). In step 3012, the image analysis system 10 may also use received supplemental patient data, received clinical data, and/or received prior procedure data.

The method 3001 may include a step 3014 of generating an artificial model of the patient's anatomy. The artificial model may visually display the determined osteophyte volume, joint-space width, B-score, and/or alignment. In step 3014, the image analysis system 10 may determine one or more modifications to make to a determined prior model to visually depict the determined osteophyte volume, joint-space width, B-score, and/or alignment and to represent the patient's anatomy shown in the one or more received images.

The method 3001 may include a step 3016 of generating an artificial model of a planned implant to be coupled to the patient's anatomy. In step 3016, the image analysis system 10 may determine a planned implant design (e.g., dimensions, thickness, type), and the generated artificial model may visually depict the planned implant design. The generated artificial model may be displayed alone and/or overlaid onto the generated artificial model of the patient's anatomy.

The method 3001 may also include a step 3018 of superimposing the artificial model of the implant onto the one or more acquired images (e.g., CT scans). The acquired image having the superimposed artificial model of the implant may also be displayed.

The method 3001 may include a step 3019 of determining a severity of osteoarthritis progression in the patient based on the determined B-score, joint-space width, osteophyte volume, and/or alignment based on the imaging data. Step 3019 may include displaying the OA severity.

One or more steps of the method 3001 may be repeated (e.g., intraoperatively). For example, step 3002 may be repeated based on intraoperatively acquired images, the determinations in step 3012 may be newly determined and/or updated, and the generated artificial models in steps 3014 and 3016 may be newly determined and/or modified. In addition, the determinations in step 3012 and the generated artificial models in steps 3014 and 3016 may be saved to a memory system (e.g., memory system 20) as prior procedure data for a future patient.

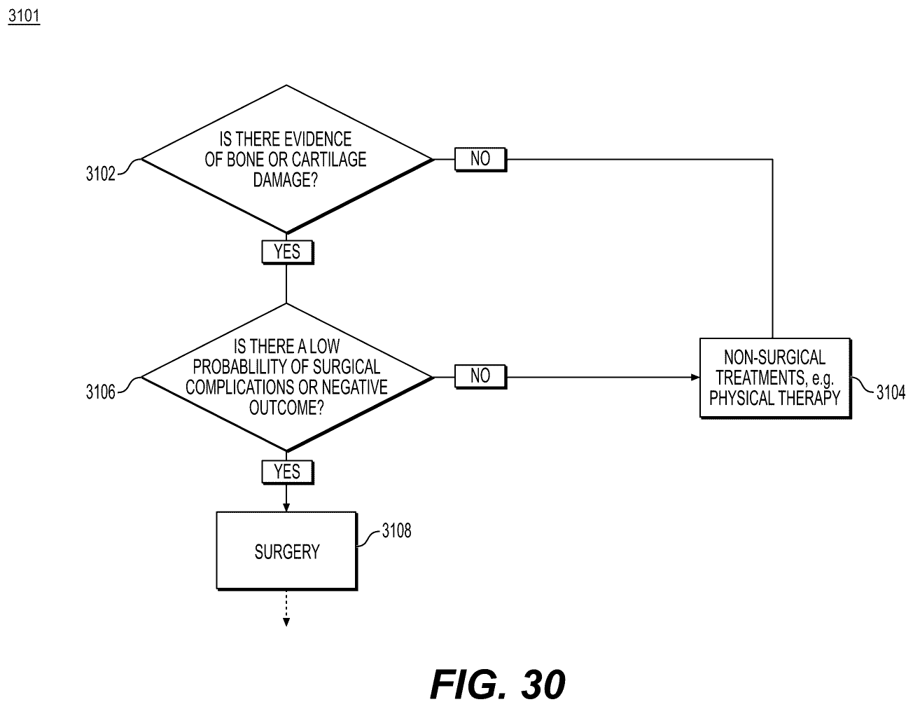

Aspects disclosed herein may be used to make a decision as to whether to proceed with surgery or to pursue less invasive treatments. FIG. 30 may illustrate an exemplary method 3101 of determining whether to proceed to surgery. However, this method 3101 is merely exemplary and not comprehensive of aspects disclosed herein. For example, the method 3101 does not include all possible decisions and does not include all possible image-based measurements. Referring to FIG. 30, the method 3101 of determining whether to proceed to surgery may include a step 3102 of determining (e.g., using the one or more algorithms 90 and/or the image analysis system 10) whether there is evidence of bone or cartilage damage. Alternatively, step 3102 may include determining whether bone or cartilage damage exceeds a predetermined damage threshold.

If it is determined in step 3102 that there is no bone or cartilage damage and/or that such damage does not exceed the predetermined damage threshold (“No” after step 3102), the method 3101 may include a step 3104 of determining and/or evaluating non-surgical treatments, such as physical therapy.

If it is determined in step 3102 that there is bone or cartilage damage and/or that such damage does exceed the predetermined damage threshold (“Yes” after step 3102), the method 3101 may include a step 3106 of determining whether a probability of surgical complications or a negative outcome is low and/or is lower than a predetermined probability. If it is determined in step 3106 that there is not a low probability and/or that the probability is not less (or alternatively, higher than) the predetermined probability (“No” after step 3106), then the method 3101 may include proceeding to step 3104 of determining and/or evaluating non-surgical treatments. If it is determined in step 3106 that there is a low probability and/or that the probability is less (or alternatively, not higher than) the predetermined probability (“Yes” after step 3106), then the method 3101 may include a step 3108 of determining and/or evaluating surgery treatment options.

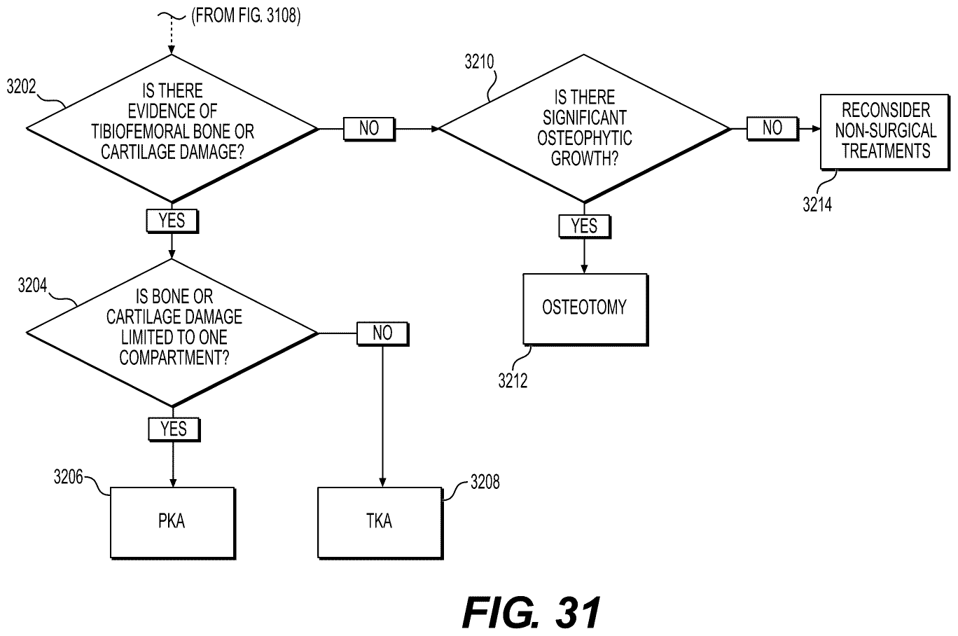

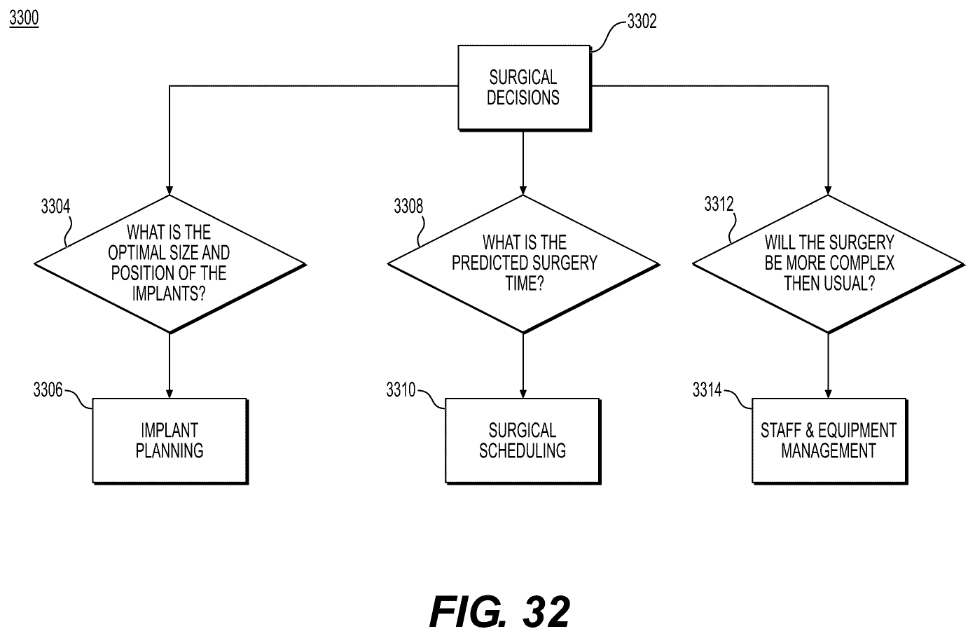

Aspects disclosed herein may be used to make a treatment decision. FIG. 32 may illustrate an exemplary method 3200 of determining a treatment. However, this method 3200 is merely exemplary and not comprehensive of aspects disclosed herein. For example, the method 3200 does not include all possible decisions and does not include all possible image-based measurements. Referring to FIG. 31, aspects disclosed herein may provide a method 3200 of determining a treatment. Method 3200 may be performed with or after method 3101. Method 3200 may include a step 3202 of determining (e.g., using the one or more algorithms 90 and/or the image analysis system 10) whether there is bone or cartilage damage in a certain area (e.g., tibiofemoral bone or cartilage damage in a knee context). Alternatively, step 3202 may include determining whether the bone or cartilage damage in the certain area exceeds a predetermined damage threshold. Step 3202 may be performed after step 3108 in method 3101. For convenience of description, a knee context will be described using tibiofemoral bone or cartilage damage.

If it is determined that there is tibiofemoral bone or cartilage damage and/or that tibiofemoral bone or cartilage damage exceeds (or is not lower than) the predetermined damage threshold (“Yes” after step 3202), then the method 3200 may proceed to step 3204 of determining whether the bone or cartilage damage is limited to one compartment. If it is determined that the bone or cartilage damage is limited to one compartment (“Yes” after step 3204), then the method 3200 may include a step 3206 of determining that a partial arthroplasty (e.g., partial knee arthroplasty) should be performed. If it is determined that the bone or cartilage damage is not limited to one compartment (“No” after step 3204), then the method 3200 may include a step 3208 of determining that a total arthroplasty (e.g., total knee arthroplasty) should be performed.

If it is determined that there is not tibiofemoral bone or cartilage damage and/or that tibiofemoral bone or cartilage damage does not exceed (or is lower than) the predetermined damage threshold (“No” after step 3202), then the method 3200 may include a step 3210 of determining whether there is significant osteophyte growth and/or whether osteophyte growth exceeds a predetermined osteophyte threshold. If it is determined, in step 3210, that there is significant osteophyte growth or that the osteophyte growth exceeds the predetermined osteophyte threshold (“Yes” after step 3210), then the method 3200 may include a step 3212 of determining that an osteotomy should be performed. If it is determined, in step 3210, that there is not significant osteophyte growth or that the osteophyte growth does not exceed the predetermined osteophyte threshold (“No” after step 3210), then the method 3200 may include a step 3214 of reconsidering non-surgical treatments.

Aspects disclosed herein may be used to make surgical decisions. FIG. 32 may illustrate an exemplary method 3300 of determining surgical decisions. However, this method 3300 is merely exemplary and not comprehensive of aspects disclosed herein. For example, the method 3300 does not include all possible decisions and does not include all possible image-based measurements. Referring to FIG. 32, the image analysis system 10 may perform various methods or decisions 3300 to arrive at surgical decisions 3302. For example, the image analysis system 10 may use the one or more algorithms 90 to determine one or more decisions 3304 relating to implant planning 3306. For example, the one or more decisions 3304 relating to implant planning 3306 may include determining an optimal size and/or position of one or more implants to be used during a procedure, but aspects disclosed herein are not limited to size and position. For example, the one or more decisions 3304 may include determinations on a type, material, thickness, etc. of one or more implants. The image analysis system 10 may use the one or more algorithms 90 to determine one or more decisions 3308 relating to surgical scheduling 3310. For example, the one or more decisions 3308 relating to implant planning 3310 may include determining a predicted procedure or surgery time or duration, but aspects disclosed herein are not limited to size and position. For example, the one or more decisions 3308 may include determinations on a room assignment, date and time, location or site, etc. The image analysis system 10 may use the one or more algorithms 90 to determine one or more decisions 3312 relating to staff and equipment management 3314. For example, the one or more decisions 3308 relating to implant planning 3310 may include determining a complexity of the procedure or surgery, but aspects disclosed herein are not limited to size and position. For example, the one or more decisions 3308 may include determinations on staff, a practitioner, surgical tools, etc.

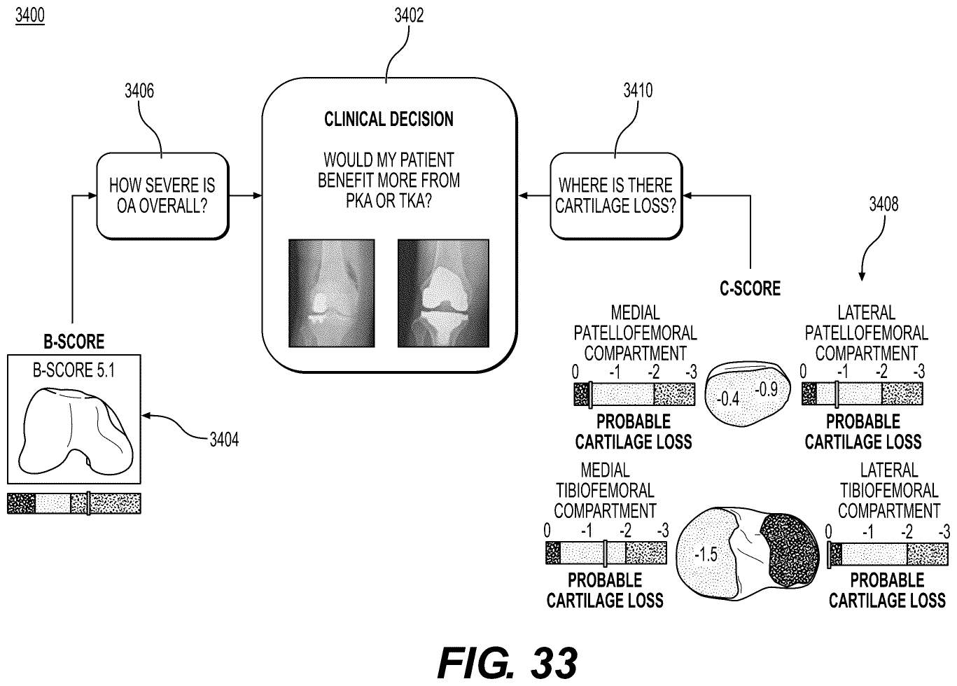

Referring to FIG. 33, aspects disclosed herein may be used to make treatment, surgical, and/or clinical decisions based on determinations of the image analysis system 10 and/or the plurality of GUIs 250. For example, determinations and displays related to B-score and C-score may be used to make a clinical decision 3402 comparing treatment options. FIG. 33 may illustrate an exemplary clinical decision 3402 and is not comprehensive of aspects disclosed herein.

The image analysis system 10 (e.g., via B-score algorithm 70) may determine a B-score of a patient's femur and a display (e.g., twelfth GUI 270, thirteenth GUI 272, fourteenth GUI 274, and/or B-score video 1606). The determined B-score, along with related GUIs and/or displays, may help a practitioner assess a severity of osteoarthritis 3406. In some examples, the image analysis system 10 may determine the severity of osteoarthritis 3406 using the one or more algorithms 90.

In addition, the image analysis system 10 (e.g., via joint space width algorithm 50) may determine a C-score of the patient's femur and a display (e.g., sixth GUI 258, seventh GUI 260, eighth GUI 262, and/or cartilage loss display 606 with gradient scale 608). The determined C-score, along with related GUIs and/or displays, may help the practitioner assess a severity and/or location of cartilage loss (and/or predicted cartilage loss) 3410. In some examples, the image analysis system 10 may determine cartilage loss 3410 using the one or more algorithms 90. The assessments by the practitioner may be used to make the clinical decision 3402, such as whether the patient would benefit more from a total knee arthroplasty or a partial knee arthroplasty. In some examples, the image analysis system 10 may automatically make the clinical decision 3402 based on determinations of the osteoarthritis severity 3406 and/or cartilage loss 3410.

### Predicted Procedures

Referring again to FIG. 2, as described above, the electronic data processing system 1 may include one or more preoperative measurement systems 100 operative to collect and/or output preoperative data 1000 regarding instant and/or prior patients. The image analysis system 10 may receive and analyze preoperative data 1000 to generate one or more outputs or determinations 2000, which may then be outputted to one or more output systems 200. For example, as described above, imaging data 1010 may be generated or otherwise collected by an imaging device 110, and patient and/or medical history data 1020 may be generated or otherwise collected by a patient or a practitioner. The image analysis system 10 may then process the imaging data 1010 and/or the patient and/or medical history data 1020 using one or more algorithms 90 (e.g., the joint-space width algorithm 50, the osteophyte detection algorithm 60, the B-Score algorithm 70, or the alignment/deformity algorithm 80) to generate one or more outputs 2000, such as a procedure time 2010, a procedure plan 2020, or a predicted outcome 2080. The image analysis system 10 may generate preoperative outputs, intraoperative outputs, and/or postoperative outputs, any or all of which may be used by patients and/or practitioners to improve patient outcomes.

As depicted in FIG. 2, in some instances, the one or more algorithms 90 includes a predicted procedure algorithm 85, with which the image analysis system 10 can generate, based on imaging data 1010, patient and/or medical history data 1020, and/or prior procedure data 1050 (e.g., predicted procedures and/or confirmed procedures), a predicted procedure 2005, which may be outputted to an output system 200 and used by a patient or a practitioner in determining what type of procedure a patient should undergo. A predicted procedure 2005 may be a type of procedure predicted or recommended for a patient. A predicted procedure 2005 may be generated based on imaging data 1010, one or more outputs of the one or more algorithms 90, patient data and/or medical history 1020, preoperative data 1000, prior procedure data 1050, one or more outputs 2000, or any combination thereof. In some instances, a predicted procedure 2005 may be selected from a set of similar or related procedures, e.g., a partial knee arthroplasty and a total knee arthroplasty, and/or a medial partial knee arthroplasty (mPKA) and a lateral partial knee arthroplasty (IPKA). However, a predicted procedure 2005 may be selected from any suitable set of procedures, or from an unbounded set of procedures. A predicted procedure 2005 may include a procedure plan 2020, as described above.

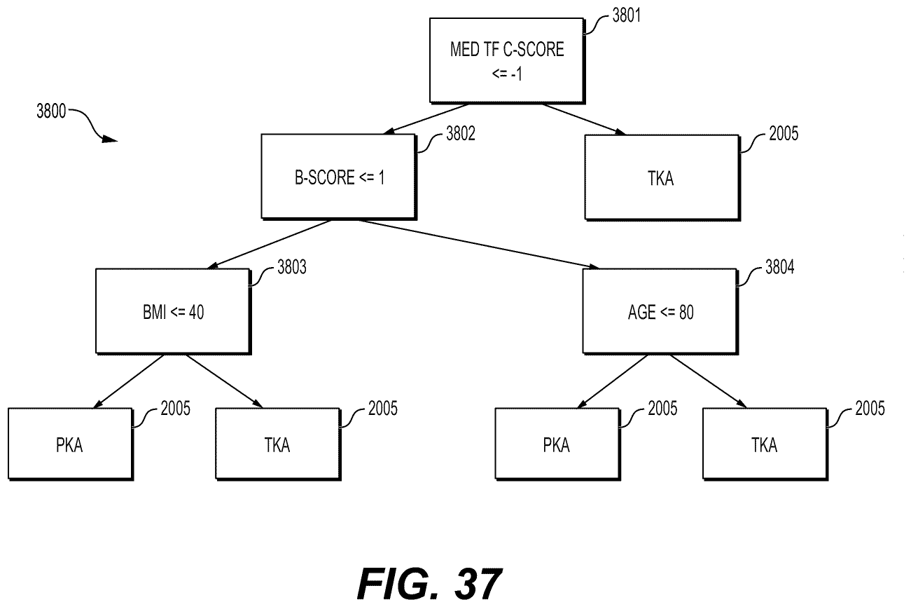

Referring to FIGS. 2 and 37, the predicted procedure algorithm 85 may, based on various inputs, generate a predicted procedure 2005 for a patient. The predicted procedure algorithm 85 may take, as an input, imaging data 1010 including one or more acquired images 302 (as described above), patient and/or medical history data 1020, and/or one or more outputs 2000 of the one or more algorithms 90. For example, patient and/or medical history data 1020 may include identity information (e.g., name, birthdate, nationality, etc.), demographic information (e.g., patient age, gender, height, weight, BMI, etc.), lifestyle information (e.g., smoking habits, exercise habits, drinking habits, eating habits, etc.), medical history information (e.g., allergies, disease progressions, addictions, prior medication use, prior drug use, prior infections, frailties, comorbidities, prior surgeries or treatment, prior injuries, prior pregnancies, utilization of orthotics, braces, prosthetics, or other medical devices, etc.), or any other suitable information, as described above. One or more outputs 2000 of the one or more algorithms 90 may include one or more B-Scores, one or more C-Scores, etc., which may be determined or generated by the one or more algorithms 90 using imaging data 1010, as described above. In some instances, the one or more algorithms 90 may be able to determine or generate a calibrated bone mineral density (BMD) for a patient, and the predicted procedure algorithm 85 may consider the BMD when generating a predicted procedure 85. For example, the BMD of a patient may influence whether a partial knee arthroplasty (PKA) or a total knee arthroplasty (TKA) is more appropriate for the patient, or whether an implant used in a PKA or a TKA for the patient should be a cemented or a cementless implant.

The predicted procedure algorithm 85 may be a machine learning algorithm that is trained using, for example, linear regression, random forest regression, CatBoost regression, statistical shape modeling (SSM), etc. Or, for example, the predicted procedure algorithm 85 may be a model produced or otherwise generated by a machine learning or artificial intelligence algorithm or model. For example, FIG. 37 depicts a predicted procedure algorithm 85 implemented as a decision tree model 3800 generated by a machine learning algorithm. In the example depicted in FIG. 37, the first node 3801 of the decision tree model 3800 considers the medial tibiofemoral C-score (“MED TF C-SCORE”) of a patient. In this example, if the medial tibiofemoral C-score of the patient is greater than −1, the decision tree model 3800 concludes by outputting a predicted procedure 2005 indicating that the patient will likely require a total knee arthroplasty (TKA). However, in this example, if the medial tibiofemoral C-score of the patient is less than or equal to −1, the decision tree model 3800 continues to the second node 3802, at which a B-score of the patient is considered. If the B-score of the patient is less than or equal to 1, the decision tree model 3800 continues to the third node 3803, at which a BMI of the patient is considered. If the BMI of the patient is less than or equal to 40, the decision tree model 3800 concludes by outputting a predicted procedure 2005 indicating that the patient will likely require a partial knee arthroplasty (PKA); if the BMI of the patient is greater than 40, the decision tree model 3800 concludes by outputting a predicted procedure 2005 indicating that the patient will likely require a TKA. However, in this example, if the B-score of the patient is greater than 1, the decision tree model 3800 continues to the fourth node 3804, at which an age of the patient is considered. If the age of the patient is less than or equal to 80, the decision tree model 3800 concludes by outputting a predicted procedure 2005 indicating that the patient will likely require a PKA; if the age of the patient is greater than 80, the decision tree model 3800 concludes by outputting a predicted procedure 2005 indicating that the patient will likely require a TKA.

The decision tree model 3800 depicted in FIG. 37 is only one example of a predicted procedure algorithm 85 and should not be considered limiting in any way. For example, a predicted procedure algorithm 85 implemented as a decision tree model may include any number of nodes considering any type of information. For example, a predicted procedure algorithm 85 implemented as a decision tree model may additionally or alternatively include a node considering a height or weight of a patient, a node considering a gender of a patient, a node considering a nationality of a patient, a node considering a lateral tibiofemoral C-score of a patient, a node considering a medial patellofemoral C-score of a patient, a node considering a lateral patellofemoral C-score of a patient, or any other node considering any other type of information. For example, a single node of a predicted procedure algorithm 85 implemented as a decision tree model may simultaneously consider two or more types of information. While the decision tree model 3800 depicted in FIG. 37 is constrained to output either PKA or TKA, a predicted procedure algorithm 85 implemented as a decision tree model may be constrained to output one of any number of procedures, or may be unconstrained, such that the decision tree model may be capable of outputting any appropriate procedure.

In some instances, a predicted procedure algorithm 85 is implemented as a logistic regression model produced or otherwise generated by a machine learning or artificial intelligence algorithm or model. For example, a predicted procedure algorithm 85 implemented as a logistic regression model in the form of 1/(1+e^{-func}), wherein func is a formula or a function including one or more variables. The one or more variables may include an age of a patient, a height or weight of a patient, a gender of a patient, a B-score of a patient, one or more C-scores of a patient, etc. The one or more variables included in the logistic regression model may be normalized against a data set of like values, e.g., such that the average value of any of the normalized variables is zero, with a standard deviation of one. The one or more variables may each be given a weight (e.g., a multiplier), such that some variables may factor into the logistic regression model more heavily than others. For example, a B-score or a C-score included in the one or more variables may be given a weight greater than that of any other variable included in the one or more variables. In one example, if the outcome of the logistic regression model, rounded to an integer, is one, then the predicted procedure algorithm 85 outputs a predicted procedure 2005 indicating that the instant patient will likely require a PKA (e.g., an mPKA); if the outcome of the logistic regression model, rounded to an integer, is zero, the predicted procedure algorithm 85 outputs a predicted procedure 2005 indicating that the instant patient will likely require a TKA. In some instances, a predicted procedure algorithm 85 implemented as a logistic regression model includes, as variables, a B-score and at least one C-score. In some instances, a predicted procedure algorithm 85 implemented as a logistic regression model includes, as variables, a plurality of C-scores, e.g., two or more of a medial tibiofemoral C-score, a lateral tibiofemoral C-score, a medial patellofemoral C-score, and a lateral patellofemoral C-score.

In some instances, when generating a predicted procedure 2005, a predicted procedure algorithm 85 may be configured to select a procedure from a set of procedures including only two procedures. For example, in some instances, when generating a predicted procedure 2005, a predicted procedure algorithm 85 may be configured to select a procedure from a set of procedures including only TKA and mPKA. In such an instance, the predicted procedure algorithm 85 may prioritize factors more closely associated with mPKAs and/or deprioritize factors less closely related with mPKAs, such as by prioritizing one or more C-scores over a B-score, or by prioritizing one or more C-scores over one or more other C-scores. For example, if the predicted procedure algorithm 85 is implemented as a decision tree model (as described above), the first node of the decision tree model may consider a C-score (e.g., a medial tibiofemoral C-score) before any subsequent node considers a B-score, or the first node of the decision tree model may consider a medial tibiofemoral C-score before any subsequent node considers any other C-score. Or for example, if the predicted procedure algorithm 85 is implemented as a logistic regression model (as described above), the logistic regression model may give a C-score (e.g., a medial tibiofemoral C-score) a weight greater than that of a B-score, or give a medial tibiofemoral C-score and a medial patellofemoral C-score greater weights than those of a lateral tibiofemoral C-score and a lateral patellofemoral C-score. However, a predicted procedure algorithm 85 may prioritize and/or deprioritize factors in any other way.

In some instances, when generating a predicted procedure 2005 for a patient, a predicted procedure algorithm 85 can consider one or more predicted procedures 2005 generated for one or more prior patients and/or one or more respective confirmed procedures 1055 received for the one or more prior patients. For example, a predicted procedure algorithm 85 may be a machine learning algorithm, or produced by a machine learning algorithm, that is trained on a procedure database including a plurality of predicted procedures 2005 generated for prior patients and respective plurality of confirmed procedures 1055 received for those prior patients. Each entry in the procedure database may include a predicted procedure 2005 generated for a prior patient, any information used in generating the predicted procedure 2005, and a confirmed procedure 1055 received for the prior patient. In some instances, after generating and outputting a predicted procedure 2005 for a patient, the image analysis system 10 can receive a confirmed procedure 1055 for the patient, such as through a GUI in which the predicted procedure 2005 is or was displayed, as described in further detail below. The predicted procedure 2005 and the corresponding confirmed procedure 1055 can then be fed back into the procedure database for further training the predicted procedure algorithm 85. By training a predicted procedure algorithm 85 on such a procedure database, for example, the predicted procedure algorithm 85 may develop various correlations or associations between individual types or groups of information or variables with various types of procedures. The predicted procedure algorithm 85 can then use these correlations and/or associations to generate ever more accurate predicted procedures 2005. For example, based on information included in the procedure database, different or additional nodes may be included in a predicted procedure algorithm 85 implemented as a decision tree model, as described above. For example, based on information included in the procedure database, different or additional variables may be included in a predicted procedure algorithm 85 implemented as a logistic regression model, or different weights may be given to one or more variables included in the logistic regression model.

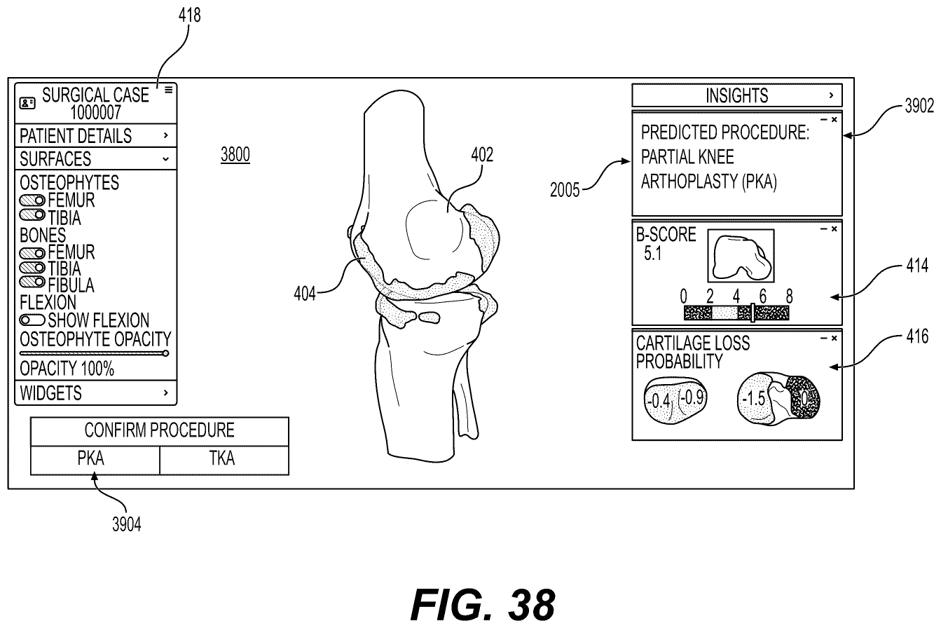

FIG. 38 illustrates a GUI 3900 provided by or otherwise communicatively coupled to the image analysis system 10, similar to the GUI 255 of FIG. 4E. GUI 3900 displays an artificial model 402 of a knee joint with indicators 404 of osteophytes, as described above. In this example, in addition to the B-score widget 414 (as described above) and the C-score widget 416 (as described above), the GUI 3900 includes a predicted procedure widget 3902 and a confirmed procedure widget 3904. As illustrated in FIG. 38, the B-score widget 414 may display various information regarding a B-score determined for the patient (e.g., by B-score algorithm 70), such as an image of a bone that represents the B-score or a gradient bar that indicates the B-score in the context of a scale, as described above. As illustrated in FIG. 38, the C-score widget 416 may display various information regarding a C-score determined for the patient (e.g., by joint-space width algorithm 50), such as an image of a bone that represents the C-score or a gradient bar that indicates the C-score in the context of a scale, as described above. Similarly, as illustrated in FIG. 38, the predicted procedure widget 3902 displays various information regarding a predicted procedure 2005 generated for the patient, such as one or more aspects of a procedure plan 2020 (e.g., a suggested implant). Selecting the predicted procedure widget 3902 may allow a user of the GUI 3900 (e.g., a practitioner) to view additional information regarding the predicted procedure 2005, such as one or more factors (e.g., variables) that contributed most significantly to the generation of the predicted procedure 2005. Selecting the predicted procedure widget 3902 may additionally or alternatively allow a user of the GUI 3900 to manipulate the predicted procedure 2005, such as by increasing/decreasing or toggling on/off one or more factors (e.g., variables) that were used in generating the predicted procedure 2005. Doing so may cause the image analysis system 10 to generate a new or updated predicted procedure 2005 for the patient and cause the new or updated predicted procedure 2005 to be displayed within the GUI 3900.

As illustrated in FIG. 38, the confirmed procedure widget 3904 may allow a user of the GUI 3900 (e.g., a practitioner) to submit a confirmed procedure 1055 for the patient to the image analysis system 10. For example, after the image analysis system 10 generates a predicted procedure 2005 for a patient (e.g., using a predicted procedure algorithm 85, as described above) and outputs the predicted procedure 2005 to be displayed within a GUI such as GUI 3900, a user of the GUI (e.g., a practitioner) may consult the predicted procedure 2005 when determining what procedure should be performed for the patient. After determining what procedure should be performed for the patient, whether or not the procedure is the same as the predicted procedure 2005, the user of the GUI may submit the procedure as a confirmed procedure 1055 to the image analysis system 10, such as through the confirmed procedure widget 3904. The image analysis system 10 may then use the predicted procedure 2005 and the corresponding confirmed procedure 1055 to further train a predicted procedure algorithm 85, as described above. For example, as illustrated in FIG. 38, the image analysis system 10 has generated a predicted procedure 2005 indicating that patient will likely require a partial knee arthroplasty (PKA) and outputted the predicted procedure 2005 for display within the predicted procedure widget 3902 of the GUI 3900. In this example, after determining what procedure should be performed for the patient, e.g., a PKA or a total knee arthroplasty (TKA), a user of the GUI 3900 can submit a confirmed procedure 1055 to the image analysis system 10 by selecting PKA or TKA, accordingly, within the confirmed procedure widget 3904.

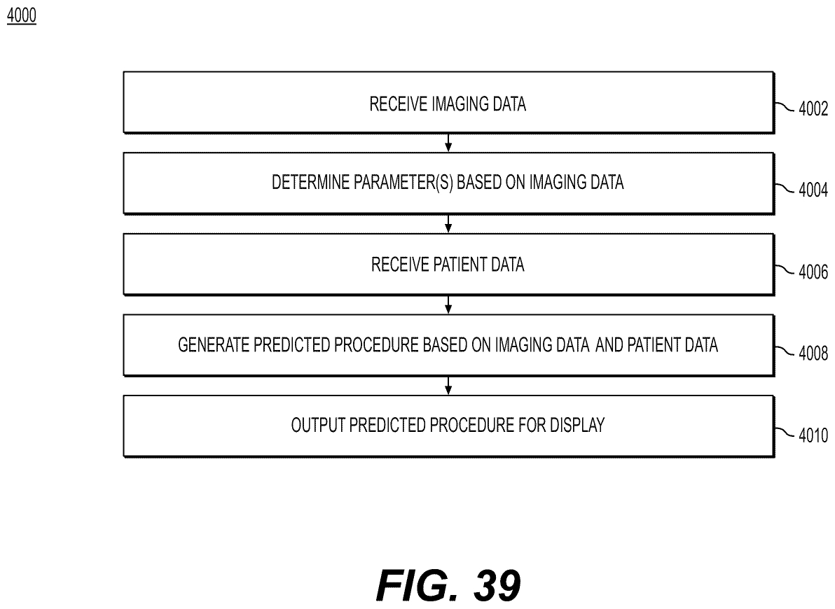

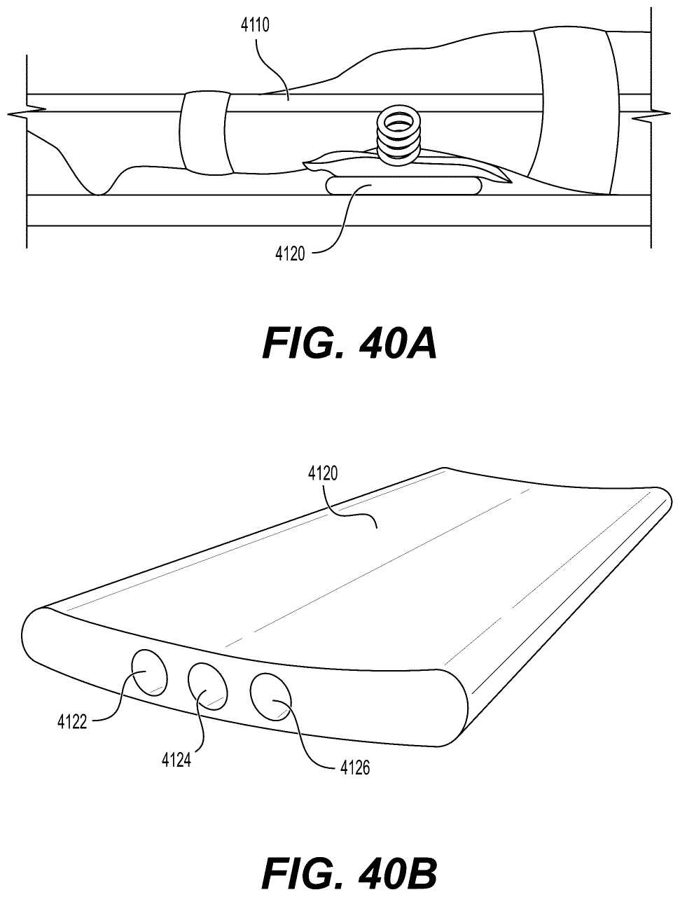

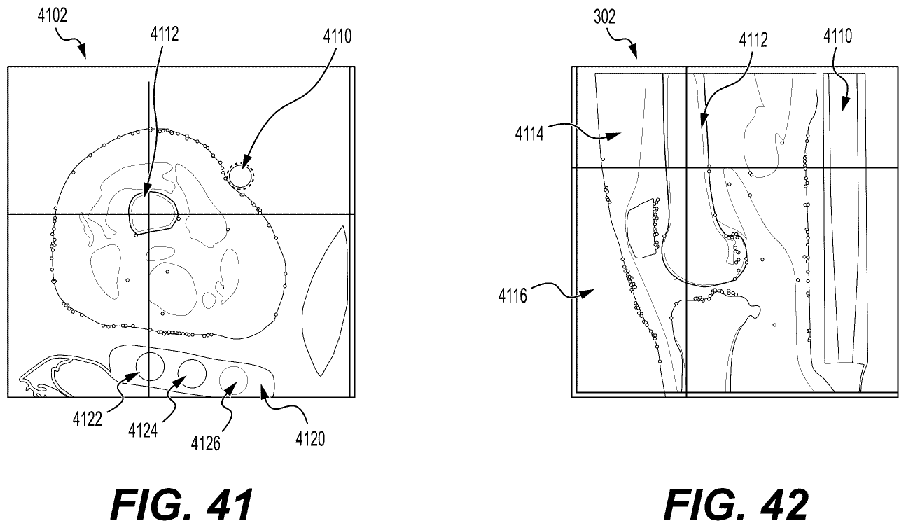

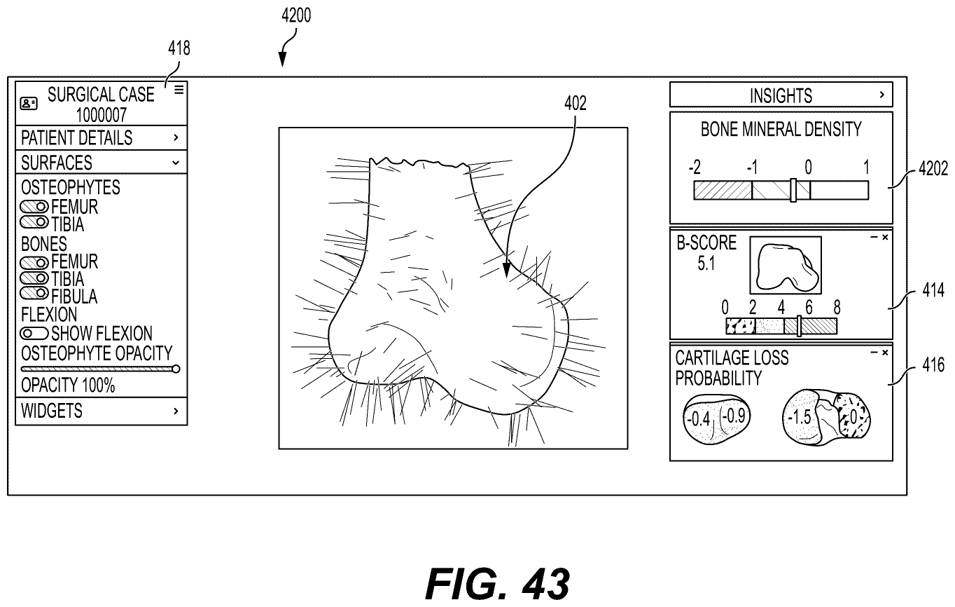

Referring to FIGS. 2 and 39, method 4000 is an exemplary method for generating a predicted procedure 2005, according to some embodiments. The method 4000 may be merely exemplary and is not comprehensive of all aspects disclosed herein. Although the steps of the method 4000 are depicted in a particular order, it should be understood that the steps of the method 4000 may occur in any order, not only in the order depicted in FIG. 39. Similarly, one or more steps of the method 4000 may occur simultaneously.

As depicted in FIG. 39, the method 4000 may begin with step 4002, in which an image analysis system 10 receives imaging data 1010 including one or more acquired images 302 of a patient's anatomy, e.g., one or more images of a patient's knee joint acquired by an imaging device 110. The imaging device 110 may be a CT imaging machine, an MRI machine, an x-ray machine, etc. and the acquired image 302 may be a CT scan, an MRI scan, an x-ray image, etc. The acquired image 302 may visualize internal structures (e.g., bone and/or tissues) of the instant patient. The image analysis system 10 may store the imaging data 1010 within a memory system 20.

As depicted in FIG. 39, after the image analysis system 10 receives the imaging data 1010, the method 4000 may continue with step 4004, in which the image analysis system 10 determines at least one parameter of the patient's anatomy based on the imaging data. The at least one parameter may include a B-score or a C-score, as described above. The at least one parameter may include both a B-score and a C-score. The at least one parameter may include a plurality of C-scores, such as two or more of a medial tibiofemoral C-score, a lateral tibiofemoral C-score, a medial patellofemoral C-score, and a lateral patellofemoral C-score. The image analysis system 10 may determine the at least one parameter using one or more algorithms 90, such as a B-score algorithm 70 or a joint-space width algorithm 50, as described above.

As depicted in FIG. 39, after the image analysis system 10 determines the at least one parameter of the patient's anatomy, the method 4000 may continue with step 4006, in which the image analysis system 10 receives patient and/or medical history data 1020. For example, patient and/or medical history data 1020 may include patient demographics, biometrics, treatment history, observations, etc. from EMR 120 and/or input (e.g., at an intake appointment) by a practitioner through an interface 130. Step 4006 may also include receiving patient information directly from the instant patient using, for example, an application through an interface 130 on a mobile device. In step 4006, the image analysis system 10 may store the patient and/or medical history data 1020 in memory system 20. However, in some instances, the method 4000 may be performed without step 4006 (e.g., the method 4000 may proceed from step 4004 directly to step 4008, as described below). For example, a predicted procedure may be derived only from data obtained via one or more medical images (e.g. acquired image 302) and the associated parameters derived from the one or more medical images.

As depicted in FIG. 39, after the image analysis system 10 receives the patient and/or medical history data 1020, the method 4000 may continue with step 4008, in which the image analysis system 10 generates a predicted procedure 2005 for the patient based on the at least one parameter of the patient's anatomy and the patient and/or medical history data 1020. For example, the image analysis system 10 may generate a predicted procedure 2005 for the patient using a predicted procedure algorithm 85, as described above. The predicted procedure algorithm 85 may be generated by a machine learning model, as described above. In some instances, the predicted procedure algorithm 85 is a decision tree model or a logistic regression model. In some instances, when generating the predicted procedure 2005, the image analysis system 10 normalizes the at least one parameter and/or the patient and/or medical history data 1020. In some instances, the predicted procedure 2005 may be either a partial knee arthroplasty (PKA) or a total knee arthroplasty (TKA). In some instances, the predicted procedure 2005 is generated at least in part using a plurality of predicted procedures generated for prior patients and a respective plurality of confirmed procedures 1055 received by the image analysis system 10 for the prior patients, as described above. In some instances, the predicted procedure 2005 includes a procedure plan 2020, e.g., a type of implant, as described above. In some instances, in step 4008, the image analysis system 10 may generate a predicted procedure 2005 without using the patient and/or medical history data 1020 (e.g., in an instance in which the method 4000 proceeds from step 4004 directly to step 4008).

As depicted in FIG. 39, after the image analysis system 10 generates a predicted procedure 2005, the method 4000 may continue with step 4010, in which the image analysis system 10 outputs the predicted procedure 2005 generated for the patient for display within a graphical user interface (GUI). In some instances, the image analysis system 10 may receive a confirmed procedure 1055 corresponding to the predicted procedure 2005 through the GUI, as described above. The image analysis system 10 may then use the predicted procedure 2005 and the corresponding confirmed procedure to further train a predicted procedure algorithm 85, as described above.

Referring to FIGS. 2 and 44, method 4500 is an exemplary method for generating a predicted procedure 2005, according to some embodiments. The method 4500 may be merely exemplary and is not comprehensive of all aspects disclosed herein. Although the steps of the method 4500 are depicted in a particular order, it should be understood that the steps of the method 4500 may occur in any order, not only in the order depicted in FIG. 44. Similarly, one or more steps of the method 4500 may occur simultaneously.

As depicted in FIG. 44, the method 400 may being with step 4502, in which an image analysis system 10 receives imaging data 1010 including one or more acquired images 302 of a patient's anatomy, e.g., one or more images of a patient's knee joint acquired by an imaging device 110. The imaging device 110 may be a CT imaging machine, an MRI machine, an x-ray machine, or any other suitable imaging matching, and the acquired image 302 may be a CT scan, an MRI scan, an x-ray image, and/or any other type of image. The acquired image 302 may visualize internal structures (E.g., bone and/or tissues) of the instant patient. The image analysis system 10 may store the imaging data 1010 within a memory system 20.

As depicted in FIG. 44, after the image analysis system 10 receives the imaging data 1010, the method 4500 may continue with step 4504, in which the image analysis system 10 identifies or determines, using the imaging data 1010 (e.g., from within one or more acquired images 302 included in the imaging data 1010), intensities of bone 4112, a reference material included in a calibration device (e.g., a single reference material included in a motion rod 4110, as described above), and at least one of fat 4114 and air 4116. In some instances, identifying or determining intensities of one or more of the bone 4112, the reference material included in the calibration device, and the at least one of the fat 4114 and the air 4116 includes identifying the bone 4112, the reference material included in the calibration device, and/or the at least one of the fat 4114 and the air 4116 using a shape model, as described above.

As depicted in FIG. 44, after the image analysis system 10 identifies or determines the intensities of the bone 4112, the reference material included in the calibration device, and the at least one of the fat 4114 and the air 4116, the method 4500 may continue with step 4506, in which the image analysis system 10 determines, based on the intensities of the bone 4112, the reference material included in the calibration device, and/or the at least one of the fat 4114 and the air 4116, a bone mineral density (BMD) of the bone 4112. In some instances, determining the BMD of the bone 4112 includes determining BMD equivalents of the reference material included in the calibration device and the at least one of the fat 4114 and the air 4116, determining a BMD calibration factor (BCF) using the BMD equivalents of the reference material included in the calibration device and the at least one of the fat 4114 and the air 4116, and determining the BMD of the bone 4112 using the BCF and the intensity of the bone 4112, as described above. The BMD equivalents of the reference material included in the calibration device and the at least one of the fat 4114 and the air 4116 may be predetermined or otherwise known to the image analysis system 10.

As depicted in FIG. 44, after the image analysis system 10 determines the BMD of the bone 4112, the method 4500 may continue with step 4508, in which the image analysis system 10 receives patient and/or medical history data 1020. For example, patient and/or medical history data 1020 may include patient demographics, biometrics, treatment history, observations, etc. from EMR 120 and/or input (e.g., at an intake appointment) by a practitioner through an interface 130. Step 4508 may also include receiving patient information directly from the instant patient using, for example, an application through an interface 130 on a mobile device. In step 4508, the image analysis system 10 may store the patient and/or medical history data 1020 in memory system 20. However, in some instances, the method 4500 may be performed without step 4508 (e.g., the method 4500 may proceed from step 4506 directly to step 4510, as described below).

As depicted in FIG. 44, after the image analysis system 10 receives the patient and/or medical history data 1020, the method 4500 may continue with step 4510, in which the image analysis system 10 generates a predicted procedure 2005 for the patient based on the BMD of the bone 4112 and the patient and/or medical history data 1020. For example, the image analysis system 10 may generate a predicted procedure 2005 for the patient using a predicted procedure algorithm 85, as described above. The predicted procedure algorithm 85 may be generated by a machine learning model, as described above. In some instances, the predicted procedure algorithm 85 is a decision tree model or a logistic regression model. In some instances, when generating the predicted procedure 2005, the image analysis system 10 normalizes the at least one parameter and/or the patient and/or medical history data 1020. In some instances, the predicted procedure 2005 may be either a partial knee arthroplasty (PKA) or a total knee arthroplasty (TKA). In some instances, the predicted procedure 2005 may include an implant fixation technique, e.g., cemented, uncemented, or hybrid. In some examples, the predicted procedure 2005 may include an indication of the type and size of an implant stem, or if additional stem extensions are required or recommended based on the BMD and/or other parameters discussed herein. For example, if a patient has a lower average BMD compared to an average of prior patients with one or more common attributes, a larger implant stem and/or additional stem extensions may be recommended for such a patient. In some instances, the predicted procedure 2005 is generated at least in part using a plurality of predicted procedures generated for prior patients and a respective plurality of confirmed procedures 1055 received by the image analysis system 10 for the prior patients, as described above. In some instances, the predicted procedure 2005 includes a procedure plan 2020, e.g., a type of implant, as described above. In some instances, the method 4500 also includes determining at least one parameter of the patient's anatomy, including at least one of a B-score or a C-score (as described above), and generating the predicted procedure 2005 based at least in part on the BMD of the bone 4112 and the at least one parameter of the patient's anatomy. The at least one parameter of the patient's anatomy may include a B-score and a C-score, or multiple C-scores, such as two or more of a medial tibiofemoral C-score, a lateral tibiofemoral C-score, a medial patellofemoral C-score, and a lateral patellofemoral C-score. In some instances, in step 4510, the image analysis system 10 may generate a predicted procedure 2005 without using the patient and/or medical history data 1020 (e.g., in an instance in which the method 4500 proceeds from step 4506 directly to step 4510).

As depicted in FIG. 44, after the image analysis system 10 generates a predicted procedure 2005, the method 4500 may continue with step 4512, in which the image analysis system 10 outputs the predicted procedure 2005 generated for the patient for display within a graphical user interface (GUI). In some instances, the image analysis system 10 may receive a confirmed procedure 1055 corresponding to the predicted procedure 2005 through the GUI, as described above. The image analysis system 10 may then use the predicted procedure 2005 and the corresponding confirmed procedure to further train a predicted procedure algorithm 85, as described above. In some instances, the method 4500 may also include generating an artificial (e.g., three-dimensional) model 402 of the patient's anatomy (e.g., the bone 4112) and outputting the artificial model for display within the GUI. The artificial model 402 of the patient's anatomy may exhibit the BMD of the bone 4112.

Aspects disclosed herein may be used to sense or collect preoperative, intraoperative, and/or postoperative information about a patient and/or a procedure.

Aspects disclosed herein contemplate implants or prosthetics, and are not limited to the contexts described. For example, implants disclosed herein may be implemented as another implant system for another joint or other part of a musculoskeletal system (e.g., hip, knee, spine, bone, ankle, wrist, fingers, hand, toes, or elbow) and/or as sensors configured to be implanted directly into a patient's tissue, bone, muscle, ligaments, etc. Each of the implants or implant systems may include sensors such as inertial measurement units, strain gauges, accelerometers, ultrasonic or acoustic sensors, etc. configured to measure position, speed, acceleration, orientation, range of motion, etc. In addition, each of the implants or implant systems may include sensors that detect changes (e.g., color change, pH change, etc.) in synovial fluid, blood glucose, temperature, or other biometrics and/or may include electrodes that detect current information, ultrasonic or infrared sensors that detect other nearby structures, etc. to detect an infection, invasion, nearby tumor, etc. In some examples, each of the implants and/or implant systems may include a transmissive region, such as a transparent window on the exterior surface of the prosthetic system, configured to allow radiofrequency energy to pass through the transmissive region. The IMU may include three gyroscopes and three accelerometers. The IMU may include a micro-electro mechanical (MEMs) integrated circuit. Implants and/or implant systems disclosed herein may also be implemented as implantable navigation systems. For example, the implants may have primarily a sensing function rather than a joint replacement function. The implants may, for example, be a sensor or other measurement device configured to be drilled into a bone, another implant, or otherwise implanted in the patient's body.

The implants, implant systems, and/or measurement systems disclosed herein may include strain gauge sensors, optical sensors, pressure sensors, load cells/sensors, ultrasonic sensors, acoustic sensors, resistive sensors including an electrical transducer to convert a mechanical measurement or response (e.g., displacement) to an electrical signal, and/or sensors configured to sense synovial fluid, blood glucose, heart rate variability, sleep disturbances, and/or to detect an infection. Measurement data from an IMU and/or other sensors may be transmitted to a computer or other device of the system to process and/or display alignment, range of motion, and/or other information from the IMU. For example, measurement data from the IMU and/or other sensors may be transmitted wirelessly to a computer or other electronic device outside the body of the patient to be processed (e.g. via one or more algorithms) and displayed on an electronic display.

Aspects and systems disclosed herein may make determinations based on images or imaging data (e.g., from CT scans). Images disclosed herein may display or represent bones, tissues, or other anatomy, and systems and aspects disclosed herein may recognize, identify, classify, and/or determine portions of anatomy such as bones, cartilage, tissue, and bone landmarks, such as each specific vertebra in a spine. Aspects and systems disclosed herein may determine relative positions, orientations, and/or angles between recognize bones, such as a Cobb angle, an angle between a tibia and a femur, and/or other alignment data.

Aspects and systems disclosed herein provide displays having graphical user interfaces configured to graphically display data, determinations, and/or steps, targets, instructions, or other parameters of a procedure, including preoperatively, intraoperatively, and/or postoperatively. Figures, illustrations, animations, and/or videos displayed via user interfaces may be recorded and stored on the memory system.

Aspects and systems disclosed herein may be implemented using machine learning technology. One or more algorithms may be configured to learn or be trained on patterns and/or other relationships across a plurality of patients in combination with preoperative information and outputs, intraoperative information and outputs, and postoperative information and outputs. The learned patterns and/or relationships may refine determinations made by one or more algorithms and/or also refine how the one or more algorithms are executed, configured, designed, or compiled. The refinement and/or updating of the one or more algorithms may further refine displays and/or graphical user interfaces (e.g., bone recognition and/or determinations, targets, recognition and/or display of other conditions and/or bone offsets, etc.).

Aspects disclosed herein may be configured to optimize a “fit” or “tightness” of an implant provided to a patient during a medical procedure based on detections by the one or more algorithms. A fit of the implant may be made tighter by aligning the implant with a shallower bone slope and/or determining a shallower resulting or desired bone slope, by increasing a thickness or other dimensions of the implant, by determining certain types of materials or a type of implants or prosthesis (e.g., a stabilizing implant, a VVC implant, an ADM implant, or an MDM implant). A thickness of the implant may be achieved by increasing (or decrease) a size or shape of the implant. Tightness may be impacted by gaps and/or joint space width, which may be regulated by an insert which may vary depending on a type of implant or due to a motion. Gaps may be impacted by femoral and tibial cuts. Tightness may further be impacted by slope. A range of slope may be based on implant choice as well as surgical approach and patient anatomy. A thickness of the implant may also be achieved by adding or removing an augment or shim. For example, augments or shims may be stackable and removable, and a thickness may be increased by adding one or more augments or shims or adding an augment or shim having a predetermined (e.g., above a certain threshold) thickness. Fit or tightness may also be achieved with certain types of bone cuts, bone preparations, or tissue cuts that reduce a number of cuts made and/or an invasiveness during surgery.

Aspects disclosed herein may be implemented during a robotic medical procedure using a robotic device. Aspects disclosed herein are not limited to specific scores, thresholds, etc. that are described. For example, outputs and/or scores disclosed herein may include other types of scores such as the hip disability and osteoarthritis score or HOOS, KOOS, SF-12, SF-36, Harris Hip Score, etc.

Aspects disclosed herein are not limited to specific types of surgeries and may be applied in the context of osteotomy procedures, computer navigated surgery, neurological surgery, spine surgery, otolaryngology surgery, orthopedic surgery, general surgery, urologic surgery, ophthalmologic surgery, obstetric and gynecologic surgery, plastic surgery, valve replacement surgery, endoscopic surgery, and/or laparoscopic surgery.

Aspects disclosed herein may improve or optimize surgery outcomes, implant designs, and/or preoperative analyses, predictions, or workflows. Aspects disclosed herein may augment the continuum of care to optimize post-operative outcomes for a patient. Aspects disclosed herein may recognize or determine previously unknown relationships, to help optimize care, predict cartilage loss or other future damage to joints, and/or to optimize design of a prosthetic.

### Appendix A

A Novel Method of Determining Bone Mineral Density from a Pre-surgical CT scans to aid in Surgical Planning

by Niall Maguire, Jared Weir, Sebastian Lustig, and Alan Brett

### Abstract

### Introduction

Patients with reduced Bone Mineral Density (BMD) may have worse outcomes after cementless joint replacement surgery. BMD phantoms may be used to calibrate Hounsfield Units (HU) to BMD values, but BMD assessment with phantoms is not common prior to surgery. We describe automated BMD calibration based on air, fat and the Aluminium motion-detection rod in Mako robotic-knee-arthroplasty CT images.

### Methods:

Image voxels containing air were identified using histogram analysis. The rod surface was identified using Active Appearance Model (AAM) search and eroded by a 4 mm radius to exclude partial voxels and beam hardening. Median voxel intensity was used as the rod HU value. Normals to the AAM segmented femur bone surface were sampled until a discontinuity was detected representing a soft-tissue boundary to air, bone or cartilage. A two-gaussian mixture was used to model histograms of fat and muscle tissue along this normal path. Mean of the lower gaussian peak was reported as fat HU value. 133 CT knee arthroplasty planning images with commercial BMD phantom (Mindways, TX) were used to determine average BMD values of air-fat-rod as calibration standards. 10 iterations of 10-fold cross-validation compared calibration from the phantom or air-fat-rod standards in tibial and femoral trabecular bone. Results:

Trabecular bone BMD from phantom calibration were highly correlated with BMD values calculated using air-fat-rod standards for femur and tibia (r≈0.98). Bland-Altman analysis showed negligible bias of ˜0.3 mg/cm3. The SD of differences is 9.9 mg/cm3, suggesting an upper bound of precision of 9 mg/cm3 (precision error for Mindways phantom is 3.6 mg/cm3), which produces a CoV=7.7% at mean BMD=117 mg/cm3.

### Conclusion:

Accurate volumetric BMD values may be derived at the knee from CT images acquired for robotic arthroplasty planning. This information may be useful in informing surgical decisions around cementless implants, particularly in patients who may be at risk of osteoporosis.

### Introduction

It is possible that patients with a reduced Bone Mineral Density (BMD) are prone to worse outcomes after joint replacement surgery [1] [2]. In particular, there is concern amongst some surgeons regarding cementless implants, because they rely on press-fit fixation in the surrounding bone for their primary stability [3]. Biomechanical studies using polyurethane bone analogues have suggested that implant stability is directly related to BMD for cementless implants. Where BMD is reduced, fixation is greatly enhanced by the addition of cement [4]. As a result, cementless implants are often only indicated for younger patients with higher BMD [5].

During knee replacement surgery (partial or total knee arthroplasty), the choice of a cemented or cementless implantation may be made by the surgeon upon bone section, by use of the so called “thumb test”. Here the strength of the trabecular bone is manually judged by pressing with the tip of the thumb [6].

Mako (Stryker, Mahwah, NJ) robotically assisted knee arthroplasty utilizes a CT scan of the joint acquired prior to surgery in order to produce a surgical plan. The acquisition protocol requires the addition of an Aluminum (Al) rod fixed to the outside of the leg in order to detection motion during scanning.

BMD measurement for the diagnosis of low bone mass and osteoporosis is usually done using dual-energy x-ray absorptiometry (DXA) scanning [7]. However, BMD may also be measured using CT scans by using internal or external calibration standards to convert CT Hounsfield Units (HU) to BMD values [8]: a technology known as Quantitative CT (QCT), which actually predates DXA scanning [9].

Therefore, it is possible that BMD calibration using the CT image acquired as part of robotic surgical planning could inform a surgeon's decision on whether to use cemented or cementless implants prior to surgery. Commercial QCT systems generally require the use of an external calibration phantom containing materials of known BMD values [10], although internal standards from the patient image such as muscle or fat have also been used [11]. However, the use of such internal references standards can be problematic because although fat is very stable as a BMD reference standard, the effective BMD value of muscle can alter with hydration state and the dynamic BMD range spanned by these tissues is limited [12].

Here we describe an automated method of BMD calibration from a CT scan acquired for robotic surgery using a mixture of two external BMD reference standards (air and the Al motion rod) to provide a wide BMD calibration range and the use of an internal reference standard (fat) as a third BMD reference value.

### Methods

### Image Data

Data were obtained from SPS data sets (Stryker Performance Solutions LLC., Chicago, Illinois, USA). They were collected from surgeons performing high-volume robotic total knee arthroplasty (TKA), or unicondylar knee arthroplasty (UKA), for knee OA using a standard CT protocol. Supine CT images were available for 6,111 knees. Because the information was de-identified, this research had an institutional review board exemption. Patients had a mean age of 67 years (range, 35 to 95) and a mean body mass index of 32 (range, 17 to 62), with 39.5% being men [13].

All knee scans were acquired using the standard Mako knee arthroplasty protocol. Subjects were positioned headfirst supine during CT acquisition, with both legs neutrally positioned and knees at full extension. The Al motion-detection rod was strapped to the affected leg to detect motion (if present, scanning was repeated). Antero-posterior and lateral views checked initial alignment. There were three CT scans performed at hip, knee, and ankle joint locations. Knee joint acquisition included coverage superior to the patella to the tibial tuberosity with contiguous 0.5-1 mm slices. Scanning parameters used a Bone reconstruction with tube cottage of 120-140 kVp and tube current of 200-400 mA with auto-exposure control. Coverage was 10 cm each side of the knee joint line. The Al motion-detection rod was secured to the lateral side of the joint. 133 of the scans from a single site contained a 3-rod BMD calibration phantom (Mindways, Austin, TX) placed under the knee during scanning (the “Phantom Set”).

### Air Calibration Standard

The calibration of CT scanners is performed so that the HU number corresponding to water is zero, and the HU number corresponding to air is −1000. CT image values are generally encoded as 12-bit integers and so fall on the range of (0-4,095), with a negative offset of 1024 included in the DICOM headers that can often be lost in anonymization. In some cases, image values are encoded as 16-bit integers. This means that HU value of air is not reliably coded at −1000. Image voxels containing air were therefore identified using histogram analysis. In general, the CT histogram has the form shown in FIG. 1.

Both air and image voxels outside the reconstructed CT filed of view (FoV) will appear black in an image, however, these voxels outside the FoV will generally be coded as the lowest HU value of −1024, but they may also be coded as 0, −8,192 or 7,191 HU. As can be seen in FIG. 1, there are also a large number of peaks at the higher HU values that are probably associated with random noise. The algorithm we developed to identify HU value of the air peak first excluded the single largest peak from the image histogram. Small, narrow peaks associated with noise were then removed from the histogram by identifying peaks that comprised ≤0.0001% of the total image volume that were ≤1 HU wide. Finally, the remaining peak with the lowest HU value was identified as the air peak.

This algorithm was tested on all 6,111 SPS CT dataset images by visual inspection of image histograms to determine which histogram peak was identified as being the air signal.

### Motion Rod Calibration Standard

To rapidly identify and segment the Al rod from the image, we developed a 3D active appearance model (AAM) (Imorphics, Manchester, UK), an established image analysis technique [14] [15] that has enabled accurate cartilage and bone segmentations in large datasets [16] [17]. For the rod model, the AAM was trained using a random sample of 24 images from the SPS CT dataset. The non-varying shape of the motion rod meant that a small training set of images could be used since the training set needed only to provide variation of image appearance surrounding the rod margin. The motion rod in each training image was manually segmented using intensity thresholding to produce surfaces for AAM training. Rod surfaces resulting from image search by AAM were eroded by a 4 mm radius to exclude partial voxels and beam hardening. To make the result robust to image noise, the median voxel intensity from the segmented rod volume was used as the rod HU value (FIG. 2). The AAM search was tested on all 6,111 CT scans and the resulting motion rod segmentation visually inspected for accuracy.

### Adipose Tissue (Fat) Calibration Standard

As part of routine Mako knee arthroplasty planning, the entire femoral and tibial bone surfaces are automatically segmented using AAMs. These surfaces may be utilised for reliable identification of soft tissue around the knee joint. Normal rays to the AAM segmented femur bone surface were projected from the centroid of each surface triangle (FIG. 2, left) and sampled until a discontinuity in image intensity values was detected representing a soft-tissue boundary to air, bone or cartilage (see FIG. 2, right).

Various methods of detecting the soft-tissue boundary were tested but we found empirically that a simple test of a HU discontinuity of +/−300 HU along the samples normal vector was sufficient to reliably detect most boundaries. This absolute jump approach would often prematurely truncate the ray within the tissue, but this is acceptable: the goal is not segmentation of the fat region, but rather acquisition of a sufficiently reliable soft tissue signal so that it may be resolved into a fat and a muscle component.

A two-gaussian mixture model (GMM) was used to resolve the histograms of soft tissue into fat and muscle tissue along these normal rays (FIG. 3). The GMM was initialised using the HU corresponding to the cumulative probability densities at 30% and 70% of the histogram. The mean value of the lower HU gaussian peak was reported as the fat HU value. An example segmentation of fat using a lower and upper HU threshold of peak +/−1 SD is shown in FIG. 4.

### Determination of BMD Calibration Values

In order to use the air-fat-rod HU values as BMD calibration standards, their BMD equivalent values need to be determined. We used the 133 CT knee arthroplasty planning images that contained a commercial BMD phantom (the Phantom Set) to determine the average BMD equivalent values of air, fat and rod as calibration standards. The three solid tubes in the phantoms were segmented in 3D (FIG. 4) with a manually thresholded region of interest (ROI) and the median from this used as the HU value for these tubes. Linear least squares fitting of the known equivalent BMD values of these tubes in mg/cm³ of was used to determine equivalent BMD values for the air, fat and Al rod standards.

### Testing the Air-Fat-Rod Calibration Standard

We used 10 iterations of 10-fold cross-validation to compare calibration from the commercial phantom tubes or air-fat-rod standards in trabecular bone at femur and tibia. Trabecular bone samples were produced from all 133 images containing a phantom by eroding the bone volume defined by the previously segmented femur and tibia surfaces by 4 mm, which is the greater than the maximum cortical thickness that might be expected in the distal femur and proximal tibia [18].

### Results

### Air Calibration Standard

The air identification algorithm worked reliably in all of the 6,111 images of our SPS test set and was notably robust to several images with partial corruption of pixel data.

### Motion Rod Standard

The motion rod algorithm successfully identified the surface of the rod in all 6,111 test images except for 2 images in which it was subsequently determined by visual inspection that the motion rod was missing.

### Adipose Tissue (Fat) Standard

The fat acquisition method was successful in all 133 phantom images, and visually verified using the histogram fits as shown in FIG. 3, as well as the manual thresholding demonstrated in figure

### Determination of Calibration Standards

FIG. 5 shows the linear fit to the known calibration standards (Tubes A, B, C) from the commercial phantom and our three image-derived calibration standards (air, fat, Al rod). The known BMD equivalent values of the three phantom rods, the derived BMD equivalent values for the air, fat and Al motion rod using the phantom values, and for comparison, the derived BMD equivalent values for the phantom tubes using our air/fat/rod standards, are shown in Table 1. These values were computed as the median values from the 133 images of the phantom set so ensure robustness against outliers.

TABLE 1
Table of HU values, BMD equivalent values of the three phantom
rods, the derived BMD equivalent values for the air, fat
and Al motion rod using the phantom values, and the derived BMD
equivalent values for the phantom tubes using our air/fat/rod
standards. BMD values in mg/cm³.

| | | HU | | |
|---|---|---|---|---|
| | Mindways | values | BMD derived | BMD derived |
| Image | BMD tube | mean | w/air/fat/rod | w/phantom |
| feature | values | (SD) | median (IQR) | median (IQR) |
| Phantom | −46.91 | −70.95 | −50.91 | — |
| tube A | | (9.63) | (−58.08, −44.31) | |
| Phantom | 151.80 | 194.83 | 147.21 | — |
| tube B | | (9.28) | (141.88, 156.50) | |
| Phantom | 379.65 | 506.85 | 382.17 | — |
| tube C | | (13.64) | (373.67, 389.12) | |
| Air | — | −994.5 | — | −742.05 |
| | | (3.02) | | (−753.15, −730.76) |
| Fat | — | −103.1 | — | −76.76 |
| | | (17.72) | | (−83.93, −70.26) |
| Motion | — | 2228.67 | — | 1669.06 |
| rod | | (32.42) | | (1643.27, 1683.68) |

### Testing the Calibration Standards

Results from the 10-fold cross-validation comparisons are shown for trabecular bone at femur and tibia in FIG. 6. Trabecular bone BMD values from phantom calibration were highly correlated with BMD values calculated using the air-fat-rod standards for both femur and tibia (r≈0.98) (FIG. 6 Left). Bland-Altman analysis showed negligible bias of ˜0.2-0.3 mg/cm³ (FIG. 6 Right) demonstrating good accuracy for our method. The SD of differences is 9.9 mg/cm³ which suggests an upper bound of short-term precision error (SD) of 9 mg/cm³ by a computation of combined variances (the precision error for Mindways phantom is 3.6 mg/cm³ [19], which produces a Coefficient of Variation (CoV)=7.7% at mean BMD=117 mg/cm³.

### Discussion

We have produced fully automated algorithms for the determination of CT image regions corresponding to air, an Aluminium motion detection rod and adipose tissue surrounding the knee joint. These algorithms have been demonstrated to be extremely robust to variability in image acquisition parameters and HU encoding in a large test set of 6,111 images.

Our use of air and Aluminium as calibration standards provides equivalent BMD values which have been used to define a linear conversion of HU to BMD equivalent values over a wide dynamic range. The use adipose tissue as a third standard with a BMD value between these two produces a more accurate linear fit during image calibration; and may also be used in error checking in the determination of these standard in the image by testing the magnitude of the three residuals of linear least squares fit. The BMD values calculated using a commercial phantom compared to our method demonstrated very good accuracy with a difference of ˜0.2-0.3 mg/cm³ which is negligible given that in the spine BMD differences between 80 and 120 mg/cm³ characterizes low bone mass (osteopenia), whereas a BMD value below 80 mg/cm³ defines osteoporosis [20].

As we have done here, it is possible to make an indirect estimate of the upper limit of the short-term precision error of our BMD calibration system. The variance in differences between the two calibration systems comes from both the statistical variation of the sampled population and from the intrinsic precision of the two systems. Assuming that the variation is dominated by measurement precision, the observed variation is the square root of the sum of the squared individual device precision values. Using the largest precision error of 3.6 mg/cm³ for phantom-based QCT BMD [19], we inferred 9 mg/cm³ to be the upper-bound or worst-case measurement precision of our BMD calibration method. Mean cancellous bone BMD in the knee is higher than that found in the vertebral bodies of the spine and so and we used the mean value at the knee of 117 mg/cm³ to calculate a CoV of 7.7%. However, it must be noted that indirect precision estimates such as this can be misleading because they include many factors such as CT scanner stability, differences in acquisition parameters and other factors that affect BMD calibration such as patient body habitus and table height [21].

The standards we have used for our BMD calibration method are a hybrid of an external phantom-based method (air and the Al rod) and an internal calibration standard method (fat). The Cov=7.7% precision of our method compares favourably to the short-term precision figure of 8.4% reported for a commercial internal calibration (“phantomless”) system using the subject's paraspinal muscle and subcutaneous fat as calibration standards [12]. We chose fat rather than muscle for our internal reference standard since it is much more stable as muscle CT numbers depend on the hydration status of the patient [12]. Blood may also be used as a stable internal calibration standard and has been used for BMD calibration at the spine [22], however the method depends on the location of a large vessel in the axial view which is more challenging at the knee than in the abdomen. As might be expected, the short-term precision of phantom-based BMD calibration methods are generally superior with CoV values of 1.4-3.6% for various commercial phantom-based systems [19]. However, both the commercial phantomless and phantom-based systems referred to here for comparison have precision that is suitable for their routine use in the clinical diagnosis and monitoring of osteoporosis.

It has been suggested that the issue of calibration might be avoided completely by using HU directly in order to compare bone quality. This is based on the argument that the regular calibration of HU on a clinical CT scanner to the standards of water at 20° C. (0 HU) and air (−1000 HU) should be sufficient since it ensures that CT images have a consistent grey scale distribution independent of a given scanner manufacturer and model, which is important for the radiologic diagnosis. However, spine phantom studies have shown that HU variation across CT scanner manufacturers and models can be as much as ˜30% [23] meaning there is considerable uncertainly in the diagnostic value of this approach.

Diagnosis of low BMD depends on comparison of values measured in a subject to normative reference values. At the spine, these have been established for the use of QCT in diagnosis of osteoporosis and low bone mass and were given above. However, unlike the wrist, hip or spine, the knee joint is not usually susceptible to osteoporotic fracture and normative values for BMD have not been established for diagnostic thresholds. Fortunately, Heiss et al has published a QCT analysis of BMD values at different depths in the femur and tibia in a cohort comprising knees of 275 (213 women, 62 men) patients with clinical and/or structural knee OA were included enrolled at five clinical centres in Europe for the “IMI-APPROACH” study [24]. BMD is thought to increase with OA status, and so the most relevant data from this study is probably the subjects with KLO (radiographically normal) knees, which represents 52 knees. These subjects were not chosen to be non-osteoporotic since osteoporosis was not explicitly excluded in the cohort definition. However, we might expect that values will probably represent a fairly normal set of BMD data for a 60+ year old population. In addition, there has been considerable research effort in measuring BMD at the knee in individuals with spinal cord injury (SCI), since individuals with SCI experience a substantial decline in bone mass in the lower extremity following their injury, predisposing them to an increased risk of low-energy, lower extremity fracture. The majority of fragility fractures among individuals with SCI occur at the distal femur or proximal tibia which means that the research done in this area may be useful in determining osteoporosis thresholds at the knee.

Cross-sectional studies have, quite consistently, demonstrated significant differences between knee BMD in people with SCI and a history of fracture and those without fractures [25]. Some of these studies suggest the existence of a fracture threshold corresponding to the value of a particular bone health measure above which fragility fractures rarely occur. Fracture thresholds in individuals with SCI have been reported using knee BMD assessed with QCT. In a study of 99 patients with motor complete SCI (21 femur or tibia fractures), a trabecular BMD of 114 mg/cm³ defined fracture threshold at the distal femur, whereas 72 mg/cm³ defined fracture threshold at the proximal tibia, both at a position 4% of the length of the bone from the articulating surface [26]. Both of these BMD fracture values are approximately 4 SD below the mean of the healthy reference cohort. This is a pattern also observed in BMD of the spine, in which the QCT guidelines [20] provide a threshold for osteoporosis as 80 mg/cm³ which is around 4 SD below the peak bone mass of healthy individuals. Using this method, a threshold could be calculated for various depths from the articulating surface in the femur of tibia using the normative data from, for example, the work of Heiss et al.

A larger normative database of BMD values in the knee would of course be preferable, as would data on the BMD differences in knees of subjects that had been diagnosed with osteoporosis. This data would allow us to produce more accurate thresholds for the determination of knees with low bone mass. Another limitation of our study is the small dataset of images containing a phantom we had available from a single site. This limits the validity of our testing since images from other sites will introduce variability in terms of CT scanner manufacturers and models, image acquisition parameters and technique. In addition, to explore short-term repeatability, we would ideally like test-retest CT image data, but this is generally not possible due to the ethical considerations of scanning a knee twice with ionizing radiation.

### CONCLUSIONS

We have demonstrated that accurate volumetric BMD values may be derived at the knee from CT images acquired for robotic arthroplasty planning. This information may be useful in the informing surgical decision about the use of cementless implants, particularly in postmenopausal women or patients that have received androgen blocking therapy as part of treatment for cancer, who may be at risk of osteoporosis.

### Acknowledgements

### Disclosures

### REFERENCES

[1] A. Aprato, S. Risitano, L. Sabatini, M. Giachino, G. Agati, and A. Massè, “Cementless total knee arthroplasty,” Ann. Transl. Med., vol. 4, no. 7, pp. 1-7, 2016, doi: 10.21037/atm.2016.01.34.
[2] M. Rathsach Andersen, N. Winther, T. Lind, H. M. Schrøder, and M. M. Petersen, “Bone remodeling of the proximal tibia after uncemented total knee arthroplasty: secondary endpoints analyzed from a randomized trial comparing monoblock and modular tibia trays-2 year follow-up of 53 cases,” Acta Orthop., vol. 90, no. 5, pp. 479-483, 2019, doi: 10.1080/17453674.2019.1637178.
[3] H. R. Mohammad, S. Campi, D. Murray, and S. Mellon, “Instruments to reduce the risk of tibial fracture following cementless unicompartmental knee replacement,” Knee, vol. 25, no. 6, pp. 988-996, December 2018, doi: 10.1016/j.knee.2018.09.001.
[4] R. W. Lee, R. G. Volz, and D. C. Sheridan, “The role of fixation and bone quality on the mechanical stability of tibial knee components,” Clin. Orthop. Relat. Res., no. 273, pp. 177-83, December 1991, [Online]. Available: http://www.ncbi.nlm.nih.gov/pubmed/1959268
[5] M. Therbo, M. M. Petersen, J.-E. Varmarken, C. A. Olsen, and B. Lund, “Influence of pre-operative bone mineral content of the proximal tibia on revision rate after uncemented knee arthroplasty,” J. Bone Joint Surg. Br., vol. 85-B, no. 7, pp. 975-979, September 2003, doi: 10.1302/0301-620X.85B7.13882.
[6] R. S. Churchill et al., “Clinical and Radiographic Outcomes of the Simpliciti Canal-Sparing Shoulder Arthroplasty System,” J. Bone Jt. Surg., vol. 98, no. 7, pp. 552-560, April 2016, doi: 10.2106/JBJS.15.00181.
[7] G. M. Blake and I. Fogelman, “The role of DXA bone density scans in the diagnosis and treatment of osteoporosis,” Postgrad. Med. J., vol. 83, no. 982, pp. 509-517, August 2007, doi: 10.1136/pgmj.2007.057505.
[8] T. M. Link, “Axial and Peripheral QCT,” in Osteoporosis and Bone Densitometry Measurements, 2013, pp. 123-134. doi: 10.1007/174_2012 729.
[9] J. E. Adams, “Quantitative computed tomography,” Eur. J. Radiol., vol. 71, no. 3, pp. 415-424, September 2009, doi: 10.1016/j.ejrad.2009.04.074.
[10] A. D. A. D. Brett and J. K. K. Brown, “Quantitative computed tomography and opportunistic bone density screening by dual use of computed tomography scans,” J. Orthop. Transl., vol. 3, no. 4, pp. 178-184, October 2015, doi: 10.1016/j.jot.2015.08.006.
[11] S. D. Boden, D. J. Goodenough, C. D. Stockham, E. Jacobs, T. Dina, and R. M. Allman, “Precise measurement of vertebral bone density using computed tomography without the use of an external reference phantom,” J. Digit. Imaging, vol. 2, no. 1, pp. 31-38, February 1989, doi: 10.1007/BF03168013.
[12] D. K. Mueller, A. Kutscherenko, H. Bartel, A. Vlassenbroek, P. Ourednicek, and J. Erckenbrecht, “Phantom-less QCT BMD system as screening tool for osteoporosis without additional radiation,” Eur. J. Radiol., vol. 79, no. 3, pp. 375-381, September 2011, doi: 10.1016/j.ejrad.2010.02.008.
[13] G. Vincent et al., “Characterizing Osteophyte Formation in Knee Osteoarthritis: Application of Machine Learning Quantification of a Computerized Tomography Cohort: Implications for Treatment,” J. Arthroplasty, pp. 1-10, May 2024, doi: 10.1016/j.arth.2024.04.083.
[14] R. H. Davies, C. J. Twining, T. F. Cootes, and C. J. Taylor, “Building 3-D Statistical Shape Models by Direct Optimization,” IEEE Trans. Med. Imaging, vol. 29, no. 4, pp. 961-981, April 2010, doi: 10.1109/TMI.2009.2035048.
[15] T. F. Cootes, G. J. Edwards, and C. J. Taylor, “Active Appearance Models,” IEEE Trans. Pattern Anal. Mach. Intell., vol. 23, no. 6, pp. 681-685, 2001, doi: 10.1109/34.927467.
[16] M. A. Bowes et al., “Machine-learning, MRI bone shape and important clinical outcomes in osteoarthritis: data from the Osteoarthritis Initiative,” Ann. Rheum. Dis., vol. 80, no. 4, pp. 502-508, April 2021, doi: 10.1136/annrheumdis-2020-217160.
[17] M. A. Bowes, G. A. Guillard, G. R. Vincent, A. D. Brett, C. B. H. Wolstenholme, and P. G. Conaghan, “Precision, Reliability, and Responsiveness of a Novel Automated Quantification Tool for Cartilage Thickness: Data from the Osteoarthritis Initiative,” J. Rheumatol., vol. 47, no. 2, pp. 282-289, February 2020, doi: 10.3899/jrheum. 180541.
[18] C. E. Taylor, H. B. Henninger, and K. N. Bachus, “Cortical and medullary morphology of the tibia,” Anat. Rec., vol. 304, no. 3, pp. 507-517, March 2021, doi: 10.1002/ar.24479.
[19] M. Bligh, L. Bidaut, R. A. White, W. A. Murphy, D. M. Stevens, and D. D. Cody, “Helical Multidetector Row Quantitative Computed Tomography (QCT) Precision,” Acad. Radiol., vol. 16, no. 2, pp. 150-159, February 2009, doi: 10.1016/j.acra.2008.08.007.
[20] American College of Radiology, “ACR-SPR-SSR PRACTICE GUIDELINE FOR THE PERFORMANCE OF QUANTITATIVE COMPUTED TOMOGRAPHY (QCT) BONE,” 2014.
[21] C. L. Brunnquell, C. Winsor, H. L. Aaltonen, and S. Telfer, “Sources of error in bone mineral density estimates from quantitative CT,” Eur. J. Radiol., vol. 144, no. September, p. 110001, November 2021, doi: 10.1016/j.ejrad.2021.110001.
[22] T. M. Keaveny et al., “Biomechanical Computed Tomography analysis (BCT) for clinical assessment of osteoporosis.,” Osteoporos. Int., vol. 31, no. 6, pp. 1025-148 June 2020, doi: 10.1007/s00198-020-05384-2.
[23] K. Engelke et al., “Clinical Use of Quantitative Computed Tomography-Based Advanced Techniques in the Management of Osteoporosis in Adults: the 2015 ISCD Official Positions-Part III,” J. Clin. Densitom., vol. 18, no. 3, pp. 393-407, July 2015, doi: 10.1016/j.jocd.2015.06.010.
[24] R. Heiss et al., “Quantitative CT of the knee in the IMI-APPROACH osteoarthritis cohort: Association of bone mineral density with radiographic disease severity, meniscal coverage and meniscal extrusion,” Bone, vol. 168, no. October 2022, p. 116673, March 2023, doi: 10.1016/j.bone.2023.116673.
[25] W. B. Edwards and T. J. Schnitzer, “Bone Imaging and Fracture Risk after Spinal Cord Injury,” Curr. Osteoporos. Rep., vol. 13, no. 5, pp. 310-317, 2015, doi: 10.1007/s11914-015-0288-6.
[26] P. Eser, A. Frotzler, Y. Zehnder, and J. Denoth, “Fracture threshold in the femur and tibia of people with spinal cord injury as determined by peripheral quantitative computed tomography,” Arch. Phys. Med. Rehabil., vol. 86, no. 3, pp. 498-504, March 2005, doi: 10.1016/j.apmr.2004.09.006.

What is claimed is:

1. A method for predicting a type of surgery for a patient, the method comprising:
receiving imaging data including at least one image acquired of a patient's anatomy, the at least one image exhibiting (i) at least one of air and/or fat, (ii) bone, and (iii) a motion rod;
identifying, using the imaging data, intensities of the bone, the motion rod, and least one of the air and the fat;
determining, based on the intensities of the bone, the motion rod, and the at least one of the air and the fat, a bone mineral density (BMD) of the bone;
receiving patient data regarding the patient;
generating, based at least in part on the BMD of the bone and the patient data, a predicted procedure for the patient; and
outputting the predicted procedure for display within a graphical user interface (GUI).

2. The method of claim 1, wherein determining the BMD of the bone comprises:
determining BMD equivalents of the motion rod and the at least one of the air and the fat;
determining a BMD calibration factor based on (a) the intensities of the bone, the motion rode, and the at least one of the air and the fat, and (b) BMD equivalents of the motion rod and the at least one of the air and the fat; and
determining the BMD of the bone based on the BMD calibration factor and the intensity of the bone.

3. The method of claim 2, further comprising:
identifying, using the imaging data, intensities of the bone, the motion rod, and the air and/or the fat;
determining BMD equivalents of the motion rod and the air and/or the fat; and
determining the BMD calibration factor based on the intensities and the BMD equivalents of the motion rod, and the air and/or the fat.

4. The method of claim 2, wherein the BMD equivalents of the motion rod and the at least one of the air and the fat are predetermined.

5. The method of claim 1, further comprising automatically identifying the motion rod, the bone, and the at least one of the air and the fat from within the at least one image.

6. The method of claim 5, wherein the motion rod, the bone, and the at least one of the air and the fat are automatically identified from within the at least one image using a shape model.

7. The method of claim 1, further comprising generating a three-dimensional model of the bone and outputting the three-dimensional model of the bone for display with the predicted procedure.

8. The method of claim 7, wherein the three-dimensional model of the bone exhibits the determined BMD of the bone.

9. The method of claim 1, further comprising determining, using the imaging data, at least one parameter of the patient's anatomy, the at least one parameter including at least one of a B-score and a C-score, and generating the predicted procedure for the patient based at least in part on the determined BMD of the bone, the patient data, and the at least one parameter.

10. The method of claim 8, further comprising outputting the determined BMD of the bone and the at least one parameter for display with the predicted procedure.

11. The method of claim 8, wherein the at least one parameter includes a B-score and a C-score.

12. The method of claim 8, wherein the at least one parameter includes a plurality of C-scores.

13. The method of claim 10, wherein the plurality of C-scores includes two or more of a medial tibiofemoral C-score, a lateral tibiofemoral C-score, a medial patellofemoral C-score, and a lateral patellofemoral C-score.
The method of claim 1, wherein the predicted procedure is either a partial knee arthroplasty or a total knee arthroplasty.

14. The method of claim 1, wherein the at least one image is taken from a computed tomography (CT) scan or a magnetic resonance imaging (MRI) scan.

15. The method of claim 1, wherein the motion rod is a bar coupled to the patient's anatomy.

16. The method of claim 16, wherein the motion rod is comprised of aluminum.

17. The method of claim 16, further comprising automatically identifying the motion rod from within the at least one image using a shape model.

18. The method of claim 1, wherein the predicted procedure is generated at least in part using a machine learning model trained with a plurality of predicted procedures generated for prior patients and a respective plurality of confirmed procedures received for the prior patients.

19. The method of claim 19, further comprising receiving a confirmed procedure for the patient and using the predicted procedure and the confirmed procedure to further train the machine learning model.
